SEQUENCE LISTING (17 sequences)

SEQ ID NO: 1 - protein, 551 aa, synthetic construct
  REGION 1..551: note = Description of Artificial Sequence: Synthetic polypeptide
MKAILVVLLYTFTTANADTLCIGYHANNSTDTVDTVLEKNVTVTHSVNLLEDKHNGKLCKLRGVAPLHLGKCNIAGWILGNPECESLSTASSWSYIVETSNSDNGTCYPGDFINYEELREQLSSVSSFERFEIFPKTSSWPNHDSNKGVTAACPHAGAKSFYKNLIWLVKKGNSYPKLSQSYINDKGKEVLVLWGIHHPSTTADQQSLYQNADAYVFVGTSRYSKKFKPEIAIRPKVRDQEGRMNYYWTLVEPGDKITFEATGNLVVPRYAFTMERNAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVGTGYIPEAPRDGQAYVRKDGEWVLLSTFLGS

SEQ ID NO: 2 - protein, 551 aa, synthetic construct
  REGION 1..551: note = Description of Artificial Sequence: Synthetic polypeptide
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFKNESFNWTGVTQNGTSSACIRGSSSSFFSRLNWLTHLNYTYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVEGYIPEAPRDGQAYVRKDGEWVLLSTFLGS

SEQ ID NO: 3 - protein, 566 aa, synthetic construct
  REGION 1..566: note = Description of Artificial Sequence: Synthetic polypeptide
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSNSSFFSRLNWLTHLNFKYPALNVTMPNNEQFDKLYIWGVHHPGTDKDQIFLYAQSSGRITVSTKRSQQAVIPNIGSRPRIRNIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISCFLLCVALLGFIMWACQKGNIRCNICI

SEQ ID NO: 4 - protein, 566 aa, synthetic construct
  REGION 1..566: note = Description of Artificial Sequence: Synthetic polypeptide
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSSSSFFSRLNWLTHLNYKYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISCFLLCVALLGFIMWACQKGNIRCNICI

SEQ ID NO: 5 - protein, 566 aa, synthetic construct
  REGION 1..566: note = Description of Artificial Sequence: Synthetic polypeptide
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSSSSFFSRLNWLTHLNYTYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISCFLLCVALLGFIMWACQKGNIRCNICI

SEQ ID NO: 6 - protein, 566 aa, synthetic construct
  REGION 1..566: note = Description of Artificial Sequence: Synthetic polypeptide
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSNSSFFSRLNWLTHLNYKYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISCFLLCVALLGFIMWACQKGNIRCNICI

SEQ ID NO: 7 - protein, 566 aa, synthetic construct; identical to SEQ ID NO: 3
  REGION 1..566: note = Description of Artificial Sequence: Synthetic polypeptide

SEQ ID NO: 8 - protein, 566 aa, synthetic construct; identical to SEQ ID NO: 6
  REGION 1..566: note = Description of Artificial Sequence: Synthetic polypeptide

SEQ ID NO: 9 - protein, 573 aa, synthetic construct
  REGION 1..573: note = Description of Artificial Sequence: Synthetic polypeptide
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSNSSFFSRLNWLTHLNFKYPALNVTMPNNEQFDKLYIWGVHHPGTDKDQIFLYAQSSGRITVSTKRSQQAVIPNIGSRPRIRNIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVEGYIPEAPRDGQAYVRKDGEWVLLSTFLGSGLNDIFEAQKIEWHEGHHHHHH

SEQ ID NO: 10 - protein, 573 aa, synthetic construct
  REGION 1..573: note = Description of Artificial Sequence: Synthetic polypeptide
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSNSSFFSRLNWLTHLNYKYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVEGYIPEAPRDGQAYVRKDGEWVLLSTFLGSGLNDIFEAQKIEWHEGHHHHHH

SEQ ID NO: 11 - protein, 573 aa, synthetic construct
  REGION 1..573: note = Description of Artificial Sequence: Synthetic polypeptide
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRGSSSSFFSRLNWLTHLNYTYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVEGYIPEAPRDGQAYVRKDGEWVLLSTFLGSGLNDIFEAQKIEWHEGHHHHHH

SEQ ID NO: 12 - protein, 573 aa, synthetic construct
  REGION 1..573: note = Description of Artificial Sequence: Synthetic polypeptide
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFKNESFNWTGVTQNGTSSACIRGSSSSFFSRLNWLTHLNYTYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVEGYIPEAPRDGQAYVRKDGEWVLLSTFLGSGLNDIFEAQKIEWHEGHHHHHH

SEQ ID NO: 13 - protein, 573 aa, synthetic construct
  REGION 1..573: note = Description of Artificial Sequence: Synthetic polypeptide
MKTIIALSYILCLVFAQKIPGNDNSTATLCLGHHAVPNGTIVKTITNDRIEVTNATELVQNSSIGEICDSPHQILDGGNCTLIDALLGDPQCDGFQNKKWDLFVERSRAYSNCYPYDVPDYASLRSLVASSGTLEFKNESFNWTGVTQNGTSSACIRGSSSSFFSRLNWLTHLNYTYPALNVTMPNKEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVEGYIPEAPRDGQAYVRKDGEWVLLSTFLGSGLNDIFEAQKIEWHEGHHHHHH

SEQ ID NO: 14 - protein, 579 aa, synthetic construct
  REGION 1..579: note = Description of Artificial Sequence: Synthetic polypeptide
MKAILVVLLYTFTTANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFINYEELREQLSSVSSFERFEIFPKTSSWPNHDSNKGVTAACPHAGAKSFYKNLIWLVKKGNSYPKLSQSYINDKGKEVLVLWGIHHPSTTADQQSLYQNADAYVFVGTSRYSKKFKPEIAIRPKVRDQEGRMNYYWTLVEPGDKITFEATGNLVVPRYAFTMERNAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYGSGYIPEAPRDGQAYVRKDGEWVLLSTFLGLNDIFEAQKIEWHEGHHHHHH

SEQ ID NO: 15 - protein, 579 aa, synthetic construct
  REGION 1..579: note = Description of Artificial Sequence: Synthetic polypeptide
MKAILVVLLYTFTTANADTLCIGYHANNSTDTVDTVLEKNVTVTHSVNLLEDKHNGKLCKLRGVAPLHLGKCNIAGWILGNPECESLSTASSWSYIVETSNSDNGTCYPGDFINYEELREQLSSVSSFERFEIFPKTSSWPNHDSNKGVTAACPHAGAKSFYKNLIWLVKKGNSYPKLSQSYINDKGKEVLVLWGIHHPSTTADQQSLYQNADAYVFVGTSRYSKKFKPEIAIRPKVRDQEGRMNYYWTLVEPGDKITFEATGNLVVPRYAFTMERNAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYGSGYIPEAPRDGQAYVRKDGEWVLLSTFLGLNDIFEAQKIEWHEGHHHHHH

SEQ ID NO: 16 - protein, 579 aa, synthetic construct
  REGION 1..579: note = Description of Artificial Sequence: Synthetic polypeptide
MKAILVVLLYTFTTANADTLCIGYHANNSTDTVDTVLEKNVTVTHSVNLLEDKHNGKLCKLRGVAPLHLGKCNIAGWILGNPECESLSTASSWSYIVETSNSDNGTCYPGDFINYEELREQLSSVSSFERFEIFPKTSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYGSGYIPEAPRDGQAYVRKDGEWVLLSTFLGLNDIFEAQKIEWHEGHHHHHH

SEQ ID NO: 17 - protein, 579 aa, synthetic construct
  REGION 1..579: note = Description of Artificial Sequence: Synthetic polypeptide
MKAILVVLLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYGSGYIPEAPRDGQAYVRKDGEWVLLSTFLGLNDIFEAQKIEWHEGHHHHHH

(19) United States
(12) Patent Application Publication
(10) Pub. No.: US 2024/0100148 A1
(43) Pub. Date: Mar. 28, 2024

(54) BROADLY REACTIVE VIRAL ANTIGENS AS IMMUNOGENS, COMPOSITIONS AND METHODS OF USE THEREOF

(71) Applicant: University of Georgia Research Foundation, Inc., Athens, GA (US)

(72) Inventors: Ted M. ROSS, Athens, GA (US); James D. ALLEN, Athens, GA (US)

(73) Assignee: University of Georgia Research Foundation, Inc., Athens, GA (US)

(21) Appl. No.: 18/528,067

(22) Filed: Dec. 4, 2023

Related U.S. Application Data
(63) Continuation of application No. PCT/US2022/032799, filed on Jun. 9, 2022.
(60) Provisional application No. 63/209,209, filed on Jun. 10, 2021.

(51) Int. Cl. A61K 39/145; A61P 31/16; A61P 37/04; C07K 14/005; C12N 7/00

(52) U.S. Cl. CPC A61K 39/145; A61P 31/16; A61P 37/04; C07K 14/005; C12N 7/00; A61K 2039/575

(57) ABSTRACT

Provided herein are non-naturally occurring, broadly reactive antigens derived from influenza viruses that are immunogenic and capable of eliciting a broadly reactive immune response, e.g., a broadly reactive neutralizing antibody response, directed against influenza virus antigens following introduction into a subject. Also provided are non-naturally, broadly reactive immunogens, vaccines, virus particles, virus-like particles (VLPs) and compositions comprising the immunogens and vaccines. Methods of generating an immune response in a human or non-human subject by administering the immunogens, vaccines, VLPs, or compositions thereof are provided. In particular, the immunogens comprise broadly reactive hemagglutinin (HA) protein antigens or soluble HA protein antigens of influenza virus strains, such as H1 or H3.

### CROSS-REFERENCE TO RELATED APPLICATIONS

This application is a continuation under 35 U.S.C. § 111(a) of PCT International Patent Application No. PCT/US2022/032799, filed Jun. 9, 2022, designating the United States and published in English, which claims priority to and the benefit of U.S. Provisional Application No. 63/209,209, filed Jun. 10, 2021, the entire contents of each of which are incorporated by reference herein.

### SEQUENCE LISTING

The present application contains a Sequence Listing which has been submitted electronically in XML format following conversion from the originally filed TXT format.

The entire contents of the electronic XML Sequence Listing, (Date of creation: Dec. 4, 2023; Size: 32,269 bytes; Name: 173093-011502US-Sequence_Listing.xml), is incorporated by reference herein.

### BACKGROUND

Influenza virus infection is an important cause of medically attended acute respiratory illness each year and imposes substantial morbidity, mortality and economic burdens both in the United States and worldwide. Shortly before the 2009 influenza pandemic, influenza-associated mortality accounted for over 611,000 years of life lost annually with an estimated cost to society of $87 billion each year. The U.S. Centers for Disease Control and Prevention estimated in 2017 that the seasonal flu vaccine was only 42% effective. This limited effectiveness was due to a mutation that occurred in the influenza A H3N2 vaccine strain that causes flu in infected individuals. In addition, cases of flu caused by influenza B viruses have risen in the time period from 2017 to 2018. Because a bad flu season can kill on the order of 50,000 people in the United States alone, new and improved immunogens and vaccines that provide broad protection against viruses, particularly, influenza virus strains in present and future circulation, are urgently required.

Infections from other virus types, such as coronaviruses, also cause serious disease and pathology in humans and in other species, for example, avian species, such as poultry. Such infections and disease adversely affect the health and performance of meat-producing and egg-producing avian animals, e.g., chickens, resulting in significant economic losses in the industry. Thus, new and improved immunogens and vaccines that provide broad protection against other virus strains, e.g., avian viruses and strains thereof, in present and future circulation, are also needed.

### SUMMARY

As described herein, non-naturally occurring, broadly reactive antigens and antigen sequences derived from influenza viruses, such as influenza H1 or H3 viruses (also referred to as “H1 or H3 influenza,” “H1 or H3 influenza viruses,” or simply, “H1 or H3” herein) are provided.

In embodiments, the non-naturally occurring, broadly reactive antigens and antigen sequences comprise amino acid sequences of HA derived from the H1 and H3 influenza viruses as provided in SEQ ID NOS: 1-17 herein. In an embodiment, the HA antigen comprises a full length HA polypeptide. In an embodiment, the HA antigen comprises a soluble HA (sHA), which lacks the transmembrane and tail domains of the HA polypeptide.

The influenza virus antigens as described herein are may be structural proteins (polypeptides) or peptides and include, for example, the hemagglutinin (HA) protein, and/or the HA1 (head) or HA2 (tail or stalk) portions (domains) which comprise the HA protein, and are potent immunogens that elicit a broadly reactive immune response in a subject against the HA protein and, ultimately, against present and future virus strains in a subject. In an embodiment, the HA antigens constitute full length HA protein or soluble HA (sHA), lacking the transmembrane (TM) and tail domains of the HA protein. As referred to herein, the virus antigens or antigen sequences (e.g., influenza virus antigens) that elicit an immune response in a subject are immunogenic antigens (i.e., immunogens). These influenza virus immunogens are termed broadly reactive, because they can elicit the production of broadly reactive antibodies that are directed against different subtypes or strains of influenza viruses having both sequence similarity and variability, and epitope (antigenic determinant) diversity in their protein antigens and sequences thereof, in particular, the HA antigen of influenza virus.

There are four different types of influenza viruses, three of which (influenza A, B, and C) infect people. Of those three infectious viruses, the influenza A and B subsets are the most common types, and each of these subsets develops different strains or subtypes. Influenza A and B viruses routinely spread in humans and cause seasonal flu epidemics. By way of nonlimiting example, the H1N1, H2N2, H3N2, and H5N1 strains are subtypes of influenza A that typically cause severe flu disease and that adapt to evade being eradicated by constantly changing their surface proteins, such as the hemagglutinin (HA) protein. Strains of influenza virus have been particularly problematic to treat because of unusually high rate of mutation and an inability to generate vaccines that were effective against the relatively rapid changes that occurred in the HA surface protein.

In an embodiment, the antigen (antigen sequence) that is immunogenic is derived from influenza A virus. In certain embodiments, the immunogen is derived from an H1 or H3 influenza virus strain or type. In an embodiment, the immunogen is derived from influenza B virus. In an embodiment, the antigen is a structural protein of the virus. In a particular embodiment, the influenza antigen is hemagglutinin (HA). In an embodiment, the HA antigen is full length HA. In an embodiment, the HA antigen is soluble HA (sHA). In other embodiments, the influenza antigen is neuraminidase (NA).

In an aspect, the non-naturally occurring influenza virus amino acid sequences and the antigens (e.g., structural antigens) comprising the sequences described herein contain broadly reactive epitopes that reflect sequence similarities and variabilities of past, present and future influenza virus antigens. Such antigen sequences and the antigens comprising the sequences are thus “non-naturally occurring, broadly reactive” antigens. The antigens are immunogenic and, when introduced into or administered to a subject, elicit broadly reactive antibodies, such as neutralizing antibodies, directed against the influenza virus, in particular, H1 or H3 influenza virus protein antigens, such as HA, or an antibody binding portion thereof, in the subject. In an embodiment, the elicited antibodies are also reactive against related, yet nonidentical H1 or H3 influenza virus types. In an embodiment, such influenza virus sequences are amino acid sequences. In an embodiment, the influenza virus sequences are polynucleotide sequences, for example, polynucleotide sequences that encode the amino acid sequences of the antigens described herein. For ease of reference, a “non-naturally occurring, broadly reactive” antigen of an influenza virus described herein is referred to as a “broadly reactive antigen.”

Accordingly, the broadly reactive influenza virus antigens described herein are immunogens as they elicit a broadly reactive immune response in a subject. The immune response is particularly in the form of a neutralizing antibody response, for example, neutralizing antibodies that are specifically directed against the HA antigen of the influenza virus and that neutralize the activity of the HA protein. Accordingly, also provided are immunogens and immunogenic compositions that contain the broadly reactive influenza virus antigens described herein, including immunogenic compositions, such as vaccines (e.g., polypeptide or polynucleotide products), that induce an immune response directed against the influenza virus, such as against the HA protein of the influenza virus, in a subject. For ease of reference, a “non-naturally occurring, broadly reactive, influenza virus immunogen” described herein will be referred to as a “broadly reactive immunogen.”

Also provided are methods of using the immunogens as described herein to induce an immune response against influenza infection, disease, and/or the symptoms thereof in a subject. In a particular embodiment, the influenza virus antigen is the HA, HA1, or HA2 protein of influenza virus type or subtype, such as the H1 or H3 influenza virus types, or a virus type related thereto, or an antibody binding portion thereof. In other embodiments, the HA protein is full length or soluble HA (sHA). Methods of using the immunogens to induce an immune response in a subject are also provided.

In an aspect, the HA immunogenic antigen has an amino acid sequence that is at least or equal to 85%, at least or equal to 90%, at least or equal to 91%, at least or equal to 92%, at least or equal to 93%, at least or equal to 94%, at least or equal to 95%, at least or equal to 96%, at least or equal to 97%, at least or equal to 98%, or at least or equal to 99% identical to an HA amino acid sequence of one or more of the HA proteins of SEQ ID NOS: 1-17 as set forth in Example 1 infra.

In an aspect, a non-naturally occurring and immunogenic influenza virus antigen, comprising an amino acid sequence that is at least 95% identical to an amino acid sequence of a hemagglutinin (HA) antigen of any one of SEQ ID NOS: 1-17 as set forth in Example 1, or an immunogenic portion thereof, is provided. In an embodiment, the influenza virus antigen comprises an amino acid sequence that is at least 98% identical to an amino acid sequence of an HA antigen as set forth in any one of SEQ ID NOS: 1-17. In an embodiment, the influenza virus antigen comprises an amino acid sequence of an HA antigen as set forth in any one of SEQ ID NOS: 1-17. In an embodiment, the influenza virus antigen consists of an amino acid sequence of an HA antigen as set forth in any one of SEQ ID NOS: 1-17. In an embodiment, the HA antigen is a full length HA protein. In an embodiment, the HA antigen is a soluble HA protein, e.g., lacking the TM and tail portions of the HA protein. In an embodiment, the influenza virus antigen comprises an amino acid sequence of a full length HA (sHA) protein antigen as set forth in SEQ ID NOS: 1-8. In an embodiment, the influenza virus antigen comprises an amino acid sequence of a soluble HA (sHA) protein antigen as set forth in SEQ ID NOS: 9-17. In an embodiment, the influenza virus is an H1 or an H3 influenza virus.

Provided in another aspect is a virus-like particle (VLP) comprising the influenza virus immunogenic antigen according to any one of the foregoing aspects. In an embodiment, the VLP comprises a polynucleotide encoding the influenza virus antigen. In an embodiment, the VLP comprises a polynucleotide encoding an influenza virus HA or sHA antigen. In an embodiment, the VLP comprises a polynucleotide encoding polypeptide comprising the amino acid sequence of any one of SEQ ID NOS: 1-17 as provided in Example 1 infra. In an embodiment, the influenza virus is an H1 or an H3 influenza virus. In an embodiment, the polynucleotide is RNA or DNA. In an embodiment, the RNA is mRNA.

In another aspect is provided a non-naturally occurring immunogen capable of generating an immune response against present and future influenza virus strains; wherein the immunogen comprises an amino acid sequence that is at least 95% identical to an amino acid sequence of a hemagglutinin (HA) antigen or sHA antigen as set forth in SEQ ID NOS: 1-17 as provided in Example 1. In an embodiment, the immunogen comprises an amino acid sequence that is at least 98% identical to an amino acid sequence of a hemagglutinin (HA) antigen or sHA antigen as set forth in SEQ ID NOS: 1-17 as provided in Example 1.

In an embodiment of any of the above-delineated aspects herein, the virus antigen, VLP, or immunogen elicits an immune response which includes the production of neutralizing antibodies. In an embodiment, the immune response includes the production of antibodies having hemagglutinin inhibitory activity and/or neuraminidase inhibitory activity. In an embodiment, the immune response further includes a cellular immune response, for example, the production of antigen-responsive T-lymphocytes.

Provided in another aspect is an immunogenic composition or vaccine comprising the influenza virus immunogen, or VLP of any of the foregoing delineated aspects and/or embodiments. In an embodiment, the immunogenic composition or vaccine comprises a pharmaceutically acceptable carrier, diluent, or excipient. In an embodiment, the immunogenic composition or vaccine further comprises an adjuvant.

Provided in another aspect is a pharmaceutically acceptable composition comprising the influenza virus antigen, immunogen, or VLP of any of the foregoing delineated aspects and/or embodiments, and a pharmaceutically acceptable carrier, diluent, or excipient. In an embodiment, the composition further comprises an adjuvant. In an embodiment, the virus or virus antigen is derived from H1 or H3 influenza virus. In an embodiment, the HA antigen or immunogen is a full length HA or a soluble influenza HA antigen or immunogen. In embodiments, the full length HA or a soluble HA influenza antigen or immunogen comprises an amino acid sequence as set forth in SEQ ID NOS: 1-17 as provided in Example 1.

Provided in another aspect is a pharmaceutically acceptable composition comprising the immunogenic composition or vaccine of the above-delineated aspect and a pharmaceutically acceptable carrier, diluent, or excipient.

Provided in another aspect is a method of generating an immune response in a subject, in which the method comprises administering to a subject in need thereof an effective amount of the virus antigen, VLP, immunogen, immunogenic composition, vaccine, or pharmaceutical composition of any of the foregoing delineated aspects and/or embodiments.

Provided in another aspect is a method of treating or protecting a subject from disease and/or the symptoms thereof, caused by influenza virus infection, the method comprising administering to the subject an effective amount of the virus antigen, VLP, immunogen, immunogenic composition, vaccine, or pharmaceutical composition of any of the foregoing delineated aspects and/or embodiments. In an embodiment of the methods, the subject is infected with influenza virus, or is at risk of or susceptible to infection by influenza virus. In an embodiment of the methods, the immune response elicited comprises the production of neutralizing antibodies and/or a cellular immune response, e.g., the production of T-lymphocytes. In an embodiment of the methods, an adjuvant is concomitantly administered to the subject. In an embodiment of the methods, the immune response is prophylactic or therapeutic. In an embodiment of the methods, an adjuvant or one or more antiviral agents is administered to the subject. In an embodiment of the methods, the subject is a human subject. In an embodiment of the methods, the subject is a non-human subject or a veterinary subject.

Provided in another aspect is a polynucleotide encoding the virus antigen, in particular, influenza HA antigen, either full length or soluble HA, of any of the foregoing aspects and delineated embodiments. In an embodiment, the polynucleotide is DNA or RNA. In an embodiment, the polynucleotide is mRNA. In an embodiment, the virus antigen is an HA protein antigen. In a particular embodiment, the HA protein antigen comprises an amino acid sequence of any one of SEQ ID NOS: 1-17 as provided in Example 1. In embodiments, the HA protein antigen is full length HA or soluble HA. In an embodiment, the polynucleotide encodes an influenza virus antigen comprising an amino acid sequence of a full length HA (sHA) protein antigen as set forth in SEQ ID NOS: 1-8. In an embodiment, the polynucleotide encodes an influenza virus antigen comprising an amino acid sequence of a soluble HA (sHA) protein antigen as set forth in SEQ ID NOS: 9-17. In an embodiment, the virus is influenza virus. In an embodiment, the influenza virus is an H1 or H3 influenza virus. In an embodiment, the above-delineated polynucleotide is contained in a composition, which includes a pharmaceutically-acceptable carrier, diluent, or excipient. In an embodiment, the above-delineated polynucleotide is contained in a virus-like particle (VLP).

In another aspect, a monovalent immunogen comprising a non-naturally occurring and immunogenic influenza virus antigen of the above-delineated aspects and/or embodiments thereof, is provided. In an embodiment, the immunogenic influenza virus antigen comprises the following non-naturally occurring, broadly reactive influenza polypeptide immunogens as described herein: Y2 comprising the sequence set forth in SEQ ID NO: 15; J1 comprising the sequence set forth in SEQ ID NOS: 3, 7, or 9; J2 comprising the sequence set forth in SEQ ID NO: 4; J3 comprising the sequence set forth in SEQ ID NO: 5; J4 comprising the sequence set forth in SEQ ID NOS: 6 or 8; NG1 comprising the sequence set forth in SEQ ID NO: 11; NG2 comprising the sequence set forth in SEQ ID NOS: 2 or 12; or NG3 comprising the sequence set forth in SEQ ID NO: 13.

In another aspect, a multivalent immunogen comprising at least two of the non-naturally occurring and immunogenic influenza virus antigens of the above-delineated aspects and/or embodiments thereof, is provided. In an embodiment, the immunogen comprises two of the non-naturally occurring and immunogenic influenza virus antigens. In embodiments, the immunogen is bivalent and comprises a combination of Y2 comprising the sequence set forth in SEQ ID NO: 15 and J4 comprising the sequence set forth in SEQ ID NOS: 6 or 8, or a combination of Y2 comprising the sequence set forth in SEQ ID NO: 15 and NG2 comprising the sequence set forth in SEQ ID NOS: 2 or 12. In an embodiment, the multivalent immunogen comprises eight of the non-naturally occurring and immunogenic influenza virus antigens described herein. In an embodiment of the foregoing monovalent or multivalent immunogens, the immunogen comprises a recombinant influenza hemagglutinin (rHA) polypeptide. In an embodiment of the foregoing monovalent or multivalent immunogens, the immunogen comprises a recombinant influenza neuraminidase (rNA) polypeptide.

In another aspect, a virus particle or virus-like particle (VLP) comprising one or more polynucleotides encoding the immunogenic influenza virus antigens of any of the above-delineated aspects and/or embodiments thereof is provided.

In another aspect, a composition comprising the monovalent or multivalent immunogen of any one of the above-delineated aspects and/or embodiments thereof is provided. In an embodiment, the composition further includes a pharmaceutically acceptable carrier, excipient, or vehicle, i.e., a pharmaceutical composition.

In an aspect, a composition comprising the virus particle or VLP of the above-delineated aspect and/or embodiments thereof is provided. In an embodiment, the composition further includes a pharmaceutically acceptable carrier, excipient, or vehicle, i.e., a pharmaceutical composition.

In an aspect, a method of treating or protecting a subject from disease and/or the symptoms thereof, caused by influenza virus infection is provided, in which the method involves administering to the subject an effective amount of the above-delineated pharmaceutically acceptable compositions comprising a monovalent or multivalent immunogen of any of the above-delineated aspects and/or embodiments thereof.

In an aspect, a method of treating or protecting a subject from disease and/or the symptoms thereof, caused by influenza virus infection is provided, in which the method involves administering to the subject an effective amount of the above-delineated pharmaceutically acceptable composition comprising a virus particle or virus-like particle (VLP) of the above-delineated aspect and/or embodiments thereof.

In an aspect, a method of generating an immune response in a subject is provided, in which the method involves administering to the subject an effective amount of the pharmaceutically acceptable composition comprising a monovalent, a multivalent immunogen, or a virus particle or a virus-like particle of any of the above-delineated aspects and/or embodiments thereof.

### Definitions

Unless defined otherwise, all technical and scientific terms used herein have the meaning commonly understood by a person skilled in the art to which the aspects and embodiments described herein pertain or relate. The following references provide one of skill with a general definition of many of the terms used in the aspects and embodiments described herein: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); Benjamin Lewin, Genes V, published by Oxford University Press, 1994 (ISBN 0-19-854287-9); Kendrew et al. (eds.); The Encyclopedia of Molecular Biology, published by Blackwell Science Ltd., 1994 (ISBN 0-632-02182-9); Molecular Biology and Biotechnology: a Comprehensive Desk Reference, Robert A. Meyers (ed.), published by VCH Publishers, Inc., 1995 (ISBN 1-56081-569-8); and Hale & Marham, The Harper Collins Dictionary of Biology (1991). As used herein, the following terms have the meanings ascribed to them below, unless specified otherwise.

By “adjuvant” is meant a substance or vehicle that non-specifically enhances the immune response to an antigen. Adjuvants may include a suspension of minerals (e.g., alum, aluminum hydroxide, or phosphate) on which antigen is adsorbed; or water-in-oil emulsion in which antigen solution is emulsified in mineral oil (e.g., Freund's incomplete adjuvant), sometimes with the inclusion of killed mycobacteria (Freund's complete adjuvant) to further enhance antigenicity. Immunostimulatory oligonucleotides (such as those including a CpG motif) can also be used as adjuvants (see, e.g., U.S. Pat. Nos. 6,194,388; 6,207,646; 6,214,806; 6,218,371; 6,239,116; 6,339,068; 6,406,705; and 6,429,199). Adjuvants also include biological molecules, such as costimulatory molecules. Exemplary biological adjuvants include, without limitation, interleukin-1 (IL-2), the protein memory T-cell attractant “Regulated on Activation, Normal T Expressed and Secreted” (RANTES), granulocyte-macrophage-colony stimulating factor (GM-CSF), tumor necrosis factor-alpha (TNF-α), interferon-gamma (IFN-γ), granulocyte-colony stimulation factor (G-CSF), lymphocyte function-associated antigen 3 (LFA-3, also called CD58), cluster of differentiation antigen 72 (CD72), (a negative regulator of B cell responsiveness), peripheral membrane protein, B7-1 (B7-1, also called CD80), peripheral membrane protein, B7-2 (B7-2, also called CD86), the TNF ligand superfamily member 4 ligand (OX40L) or the type 2 transmembrane glycoprotein receptor belonging to the TNF superfamily (4-1BBL).

By “administer” is meant giving, supplying, dispensing, delivering, or applying a composition, agent, therapeutic and the like to a subject, or applying or bringing the composition and the like into contact with the subject. Administering or administration may be accomplished by any of a number of routes, such as, for example, without limitation, topical, oral, subcutaneous, intramuscular, intraperitoneal, intravenous (IV), (injection), intrathecal, intramuscular, dermal, intradermal, intracranial, inhalation, rectal, intravaginal, or intraocular.

By “agent” is meant any small molecule, small molecule chemical compound, antibody, nucleic acid molecule, peptide, polypeptide, or fragments thereof.

By “alteration” is meant a change (increase or decrease) in the expression levels or activity of a gene or polypeptide as detected by standard art known methods such as those described herein. As used herein, an alteration includes a 5% change in expression levels, a 10% change in expression levels, preferably a 25% change, more preferably a 40% change, and most preferably a 50% or greater change in expression levels.

By “ameliorate” is meant decrease, reduce, diminish, suppress, attenuate, arrest, or stabilize the development or progression of a disease or pathological condition.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “antibody” is meant an immunoglobulin (Ig) molecule produced by B lymphoid cells and having a specific amino acid sequence. Antibodies are evoked or elicited in subjects (humans or other animals or mammals) following exposure to a specific antigen (immunogen). A subject capable of generating antibodies/immunoglobulin (i.e., an immune response) directed against a specific antigen/immunogen is said to be immunocompetent. Antibodies are characterized by reacting specifically with (e.g., binding to) an antigen or immunogen in some demonstrable way, antibody and antigen/immunogen each being defined in terms of the other.

“Eliciting an antibody response” refers to the ability of an antigen, immunogen or other molecule to induce the production of antibodies. Antibodies are of different classes, e.g., IgM, IgG, IgA, IgE, IgD and subtypes or subclasses, e.g., IgG1, IgG2, IgG2a, IgG2b, IgG3, IgG4. An antibody/immunoglobulin response elicited in a subject can neutralize a pathogenic (e.g., infectious or disease-causing) agent by binding to epitopes (antigenic determinants) on the agent and blocking or inhibiting the activity of the agent, and/or by forming a binding complex with the agent that is cleared from the system (or body) of the subject, e.g., via the liver.

As used herein, “broadly reactive” means that an immune response is elicited against a pathogen-derived antigen protein (e.g., a virus protein sequence, such as HA or NA) in a subject that is sufficient to block, inhibit, impede, neutralize, or prevent infection of a broad range of related pathogens (such as most or all influenza viruses within a specific subtype). In an embodiment, the subject is a mammalian subject. In an embodiment, the subject is an avian subject.

By “antigen” is meant a compound, composition, or substance that can stimulate the production of antibodies or a T-cell response in an animal, including compositions that are injected or absorbed into an animal. An antigen reacts with the products of specific humoral or cellular immunity, including those induced by heterologous immunogens. In some embodiments of the disclosed compositions and methods, the antigen is an influenza hemagglutinin (HA) protein. In an embodiment, the HA protein is full length HA. In an embodiment, the HA protein is sHA, which is truncated and does not include a transmembrane domain and a tail domain. In many cases, an antigen that elicits or stimulates an immune response in a subject is termed an “immunogen.” In specific embodiments, the HA protein comprises an amino acid sequence as provided in SEQ ID NOS: 1-17 (Example 1).

The term “antigenic drift” refers to a mechanism for variation in organisms or microorganisms such as viruses that involves the accumulation of mutations within the genes that code for antibody-binding sites (also called antigenic determinants or epitopes). This process results in a new strain of virus/virus particles that is not inhibited or blocked as effectively by antibodies that were originally generated against the antigens of virus strains prior to mutation, thus allowing the virus to spread more easily throughout a partially immune population. By way of example, antigenic drift occurs in both influenza A and influenza B viruses.

In the context of a live virus, the term “attenuated” reflects a virus that is attenuated if its ability to infect a cell or subject and/or its ability to produce disease is reduced (for example, diminished, abrogated, or eliminated) compared to the ability of a wild-type virus to produce disease in the subject. Typically, an attenuated virus retains at least some capacity to elicit an immune response following administration to an immunocompetent subject. In some cases, an attenuated virus can elicit a protective immune response without causing any signs or symptoms of infection. In some embodiments, the ability of an attenuated virus to cause disease or pathology in a subject is reduced at least about or equal to 5%, or at least about or equal to 10%, or at least about or equal to 25%, at least about or equal to 50%, at least about or equal to 75%, or at least about or equal to 80%, or at least about or equal to 85%, or at least about or equal to 90%, or at least about or equal to 95%, or greater, relative to the ability of a wild-type virus to cause disease or pathology in the subject.

The term “clade” refers to the different categorizations (often called subtypes) of the known influenza viruses, such as, e.g., the influenza A H3N2 virus. By way of example, viruses in an H3N2 clade are genetically related, but do not share the exact viral genome. As appreciated by the skilled practitioner, there are many clades and subclades of H3N2 virus subtypes designated in the art. By way of example, one clade is 3C.2a; subclades of this clade include 3C.2a.1, 3C.2a.2, 3C.2a.3 and 3C.2a.4. In addition, there are at least ten different clades of H5N1 virus subtypes designated in the art: clade 0 clade 1, clade 2, clade 3, clade 4, clade 5, clade 6, clade 7, clade 8 and clade 9 (Abdel-Ghafar et al., N Engl J Med 358:261-273, 2008). Clade 2 is further divided into sub-clades (including clade 2.1, clade 2.2, clade 2.3, clade 2.4 and clade 2.5).

A “codon-optimized” nucleic acid refers to a nucleic acid sequence that has been altered such that the codons are optimal for expression in a particular system (such as a particular species or group of species). For example, a nucleic acid sequence can be optimized for expression in mammalian cells. Codon optimization does not alter the amino acid sequence of the encoded protein.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of an analyte, compound, agent, or substance to be detected. By “detectable label” is meant a composition that, when linked to a molecule of interest, renders the latter detectable, e.g., via spectroscopic, photochemical, biochemical, immunochemical, or chemical means. Nonlimiting examples of useful detectable labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition, disorder, or pathology that damages or interferes with the normal function of a cell, tissue, or organ. Examples of diseases include those caused by influenza virus infection and the symptoms and adverse effects that are caused by infection of the body with the H1 or H3 influenza virus. Influenza virus causes flu and its symptoms in infected individuals.

By “effective amount” is meant the amount of an active therapeutic agent, composition, compound, biologic (e.g., a vaccine or therapeutic peptide, polypeptide, or polynucleotide) required to ameliorate, reduce, improve, abrogate, diminish, or eliminate the symptoms and/or effects of a disease, condition, or pathology relative to an untreated patient. In one embodiment, an effective amount is the amount of an antigen required to elicit an immune response. The effective amount of an immunogen or a composition comprising the immunogen, as used to practice the methods of therapeutic treatment of a disease, condition, or pathology, varies depending upon the manner of administration, the age, body weight, and general health of the subject. Ultimately, the attending physician or veterinarian will decide the appropriate amount and dosage regimen. Such amount is referred to as an “effective” amount.

A “therapeutically effective amount” refers to a quantity of a specified agent sufficient to achieve a desired effect in a subject being treated with that agent. For example, this may be the amount of an influenza virus immunogen or vaccine useful for eliciting an immune response in a subject and/or for preventing infection by influenza virus. Ideally, in the context of the present disclosure, a therapeutically effective amount of an influenza vaccine or immunogenic composition is an amount sufficient to increase resistance to, prevent, ameliorate, reduce, and/or treat infection caused by influenza virus in a subject without causing a substantial cytotoxic effect in the subject. The effective amount of an immunogenic composition (or vaccine) useful for increasing resistance to, preventing, ameliorating, reducing, and/or treating infection in a subject depends on, for example, the subject being treated, the manner of administration of the therapeutic composition and other factors, as noted supra.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. A portion or fragment of a polypeptide may be a peptide. In the case of an antibody or immunoglobulin fragment, the fragment typically binds to the target antigen.

By “fusion protein” is meant a protein generated by expression of a nucleic acid (polynucleotide) sequence engineered from nucleic acid sequences encoding at least a portion of two different (heterologous) proteins or peptides. To create a fusion protein, the nucleic acid sequences must be in the same reading frame and contain no internal stop codons. For example, a fusion protein includes an influenza HA protein or NA protein fused to a heterologous protein.

By “genetic vaccine” is meant an immunogenic composition comprising a polynucleotide encoding an antigen.”

By “virus polypeptide,” such as an H1 or an H3 influenza virus, is meant an amino acid sequence that is at least 85% identical, or at least 95% or greater identical, to an amino acid sequence of an antigen, e.g., an HA protein as set forth in SEQ ID NOS: 1-17 infra, or a fragment thereof capable of inducing an immune response against the virus, virus infection, and/or the symptoms thereof in an immunized subject. In embodiments, an influenza virus polypeptide comprises or consists of the amino acid sequences or a fragment thereof as described herein and provided in SEQ ID NOS: 1-17 infra.

By “virus polynucleotide” is meant a nucleic acid molecule encoding an influenza virus polypeptide, such as an H1 or an H3 influenza virus, (antigen or antigen protein), as described herein. In an embodiment, a polynucleotide is a DNA or RNA polynucleotide. In an embodiment, the polynucleotide is mRNA.

The term “Hemagglutinin (HA)” refers to a surface glycoprotein expressed by an influenza virus. HA mediates binding of the virus particle to a host cell and subsequent entry of the virus into the host cell. The nucleotide and amino acid sequences of numerous influenza HA proteins are known in the art and are publicly available, such as those deposited in the publicly accessible GenBank (NCBI) and UniProtKB databases. By way of nonlimiting example, a list of GenBank Accession Nos. of H5N1 HA sequences may be found in US Patent Application Publication US 2015/0030628. A nonlimiting example of the amino acid sequence of the HA protein of influenza A virus (strain A/Puerto Rico/8/1934 H1N1) is provided under UniProtKB Accession No. P03452 (HEMA_134A1). A nonlimiting example of the amino acid sequence of the HA protein of an influenza A, H3N2 virus, (A/Hong Kong/1-4/1968(H3N2), is provided under Accession Number CY033017. HA (along with neuraminidase (NA)) is one of the two major influenza virus antigenic proteins having antigenic determinants (epitopes) that are recognized and bound by antibodies/immunoglobulins. In embodiments, HA is HA1 (H1) or HA2 (H2).

In embodiments, an HA protein or fragment thereof may have at least about or equal to 85%, or at least about or equal to 90%, 95%, 98%, 99%, or greater, amino acid sequence identity to the amino acid sequence of a representative influenza A virus HA protein or a fragment thereof.

In an embodiment, the HA immunogenic antigen is an H1 or H3 HA protein, which may be full length or a soluble form thereof, which comprises or consists of the amino acid sequences set forth in Example 1, infra.

“Hybridization” means hydrogen bonding, which may be Watson-Crick, Hoogsteen, or reversed Hoogsteen hydrogen bonding, between complementary nucleobases. For example, in DNA, adenine and thymine, and cytosine and guanine, are, respectively, complementary nucleobases that pair through the formation of hydrogen bonds.

The term “immune response” is meant any response mediated by an immunoresponsive cell. In one example of an immune response, leukocytes are recruited to carry out a variety of different specific functions in response to exposure to an antigen (e.g., a foreign entity). Immune responses are multifactorial processes that differ depending on the types of cells involved. Immune responses include cell-mediated responses (e.g., T cell responses), humoral responses (B cell/antibody responses), innate responses and combinations thereof.

By “immunogen” is meant a compound, composition, or substance which, under appropriate conditions, can elicit or stimulate an immune response, such as the production of antibodies, and/or a T-cell response, in an animal, including compositions that are injected into or otherwise delivered to an animal. As used herein, an “immunogenic composition” is a composition comprising an immunogen (such as an HA polypeptide or peptide (e.g., a full length or a soluble form of an HA polypeptide) or a polynucleotide encoding such immunogen) or a vaccine comprising an HA polypeptide or peptide (e.g., a full length or a soluble form of an HA polypeptide) or a polynucleotide encoding such immunogen). As will be appreciated by the skilled person in the art, if administered to a subject in need prior to the subject's contracting disease or experiencing full-blown disease, an immunogenic composition can be prophylactic and result in the subject's eliciting an immune response, e.g., a neutralizing antibody and/or cellular immune response, to protect against disease, or to prevent more severe disease or condition, and/or the symptoms thereof. If administered to a subject in need following the subject's contracting disease, an immunogenic composition can be therapeutic and result in the subject's eliciting an immune response, e.g., a neutralizing antibody and/or cellular immune response, to treat the disease, e.g., by reducing, diminishing, abrogating, ameliorating, abating, alleviating, or eliminating the disease, and/or the symptoms thereof. In an embodiment, the immune response is a B cell response, which results in the production of antibodies, e.g., neutralizing antibodies, directed against the immunogen or immunogenic composition comprising the antigen or antigen sequence. In a manner similar to the foregoing, in some embodiments, an immunogen, immunogenic composition, or vaccine can be prophylactic. In some embodiments, an immunogen, immunogenic composition, or vaccine can be therapeutic. In an embodiment, the disease is influenza (flu). In an embodiment, the disease is infectious bronchitis. In some cases herein, the terms immunogen and vaccine are used interchangeably.

By “immunogenic composition” is meant a composition comprising an antigen, antigen sequence, or immunogen, wherein the composition elicits an immune response in an immunized subject.

The term “immunize” (or immunization) refers to rendering a subject protected from, or immunologically responsive to, a disease or pathology caused by a pathogenic agent, e.g., an infectious disease caused by a virus, e.g., influenza virus H1 or H3, such as by vaccination. In some cases herein, the terms “immunization” and “vaccination” may be used interchangeably (e.g., immunization/vaccination).

The term “Influenza virus” refers to a segmented negative-strand RNA virus that belongs to the Orthomyxoviridae family of viruses. There are three types of Influenza viruses: A, B and C. Influenza A viruses infect a wide variety of birds and mammals, including humans, horses, marine mammals, pigs, ferrets, and chickens. In animals, most influenza A viruses cause mild localized infections of the respiratory and intestinal tract. However, highly pathogenic influenza A strains, such as, without limitation, H5N1, H5N2, H5N6, H5N8, H7N9, H9N2, H1N1, H1N2, H2N1, H2N2, H2N3, H7N3, H7N7, H3N2, H3N1, and related viruses, cause systemic infections in poultry in which mortality may reach 100%. H5N1 is also referred to as “avian influenza.”

By “inhibitory nucleic acid” is meant a double-stranded RNA, siRNA, shRNA, or antisense RNA, or a portion thereof, or a mimetic thereof, that when administered to a mammalian cell results in a decrease (e.g., by 5%, 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid, protein, or peptide is purified if it is substantially free of cellular material, debris, non-relevant viral material, or culture medium when produced by recombinant DNA techniques, or of chemical precursors or other chemicals when chemically synthesized. Purity and homogeneity are typically determined using standard purification methods and analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified. The term “isolated” also embraces recombinant nucleic acids, proteins or viruses, as well as chemically synthesized nucleic acids or peptides.

By “isolated polynucleotide” is meant a nucleic acid (e.g., a DNA molecule) that is free of the genes which flank the gene in the naturally-occurring genome of the organism from which the nucleic acid molecule of the described aspects and embodiments is derived. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule (e.g., mRNA), as well as a recombinant DNA that is part of a hybrid gene encoding additional polypeptide sequence.

By an “isolated polypeptide” is meant a polypeptide, such as described herein, that has been separated from components that naturally accompany it. Typically, the polypeptide is isolated when it is at least 30% by weight, at least 40%, by weight, at least 50%, by weight, at least 60%, by weight, free from the proteins and naturally-occurring organic molecules with which it is naturally associated. Preferably, an isolated polypeptide preparation is at least 75%, at least 90%, or at least 99%, by weight, free from the proteins and naturally-occurring organic molecules with which it is naturally associated. An isolated polypeptide may be obtained, for example, by extraction from a natural source; by expression of a recombinant nucleic acid encoding such a polypeptide; or by chemically synthesizing the protein. Purity can be measured by any standard, appropriate method, for example, column chromatography, polyacrylamide gel electrophoresis, or by HPLC analysis. An isolated polypeptide can refer to broadly active virus immunogen polypeptide generated by the methods described herein.

By “linker” is meant one or more amino acids that serve as a spacer between two polypeptides or peptides of a fusion protein.

By “marker” is meant any protein or polynucleotide having an alteration (e.g., increase or decrease) in expression level or activity that is associated with a disease, condition, pathology, or disorder.

A “Matrix (M1) protein” refers to an influenza virus structural protein found within the viral envelope. M1 is thought to function in assembly and budding of virus following infection of a cell.

As used herein, “obtaining” as in “obtaining an agent” includes synthesizing, isolating, purifying, purchasing, or otherwise acquiring the agent.

The term “operably linked” refers to nucleic acid sequences as used herein. By way of example, a first nucleic acid sequence is operably linked to a second nucleic acid sequence when the first nucleic acid sequence is placed in a functional relationship with the second nucleic acid sequence. For instance, a promoter is operably linked to a coding sequence if the promoter affects (allows) the transcription or expression of the coding sequence. Generally, operably linked DNA sequences are contiguous and, where necessary to join two protein-coding regions, are in the same reading frame.

The nucleotide sequence encoding an HA protein that is broadly reactive (such that an immune response (e.g., an antibody response) is generated against it as well as other, perhaps antigenically drifted, yet related viruses) can be optimized for expression in mammalian cells via codon-optimization and RNA optimization (such as to increase RNA stability) using procedures and techniques practiced in the art.

A (non-naturally occurring) broadly reactive, immunogenic antigen, such as influenza (e.g., H1 or H3 influenza virus) hemagglutinin (HA) protein, for eliciting an immune response in a subject possesses a collective set of strongly immunogenic epitopes (also called antigenic determinants). An influenza virus HA protein described herein is suitable for use as an immune response-eliciting immunogen, or vaccine, which elicits a broadly reactive immune response, e.g., a neutralizing antibody response, against other related, but nonidentical, virus types which express HA proteins on the viral surface, when introduced into a host subject, in particular, a human subject infected with influenza H1 or H3 virus. The immunogenic antigen (or vaccine) is advantageous for providing an anti-virus immunogen (or a vaccine) that elicits a broadly active immune response against other influenza virus HA antigens, such as H1 or H3, with antigenic variability and similarity, and treats or protects against infection and disease caused by more than one H1 or H3 influenza virus subtype.

By “open reading frame (ORF)” is meant a series of nucleotide triplets (codons) that code for amino acids without any termination codons. These sequences are usually translatable into a peptide or polypeptide.

The term “pharmaceutically acceptable vehicle” refers to conventional carriers (vehicles) and excipients that are physiologically and pharmaceutically acceptable for use, particularly in mammalian, e.g., human, subjects, as well as in other animal or avian subjects. Such pharmaceutically acceptable vehicles are known to the skilled practitioner in the pertinent art and can be readily found in Remington's Pharmaceutical Sciences, by E. W. Martin, Mack Publishing Co., Easton, Pa., 15th Edition (1975) and its updated editions, which describes compositions and formulations suitable for pharmaceutical delivery of one or more therapeutic compositions, such as one or more influenza HA immunogens (vaccines), and additional pharmaceutical agents. In general, the nature of a pharmaceutically acceptable carrier depends on the particular mode of administration being employed. For instance, parenteral formulations usually comprise injectable fluids/liquids that include pharmaceutically and physiologically acceptable fluids such as water, physiological saline, balanced salt solutions, aqueous dextrose, glycerol or the like as a vehicle or diluent. For solid compositions (for example, powder, pill, tablet, or capsule forms), conventional non-toxic solid carriers may include, for example, pharmaceutical grades of mannitol, lactose, starch, or magnesium stearate, which typically stabilize and/or increase the half-life of a composition or drug. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

By “plasmid” (or “vector”) is meant a circular nucleic acid molecule capable of autonomous replication in a host cell.

By “polypeptide” (or protein) is meant a polymer in which the monomers comprise amino acid residues that are joined together through amide bonds. When the amino acids are alpha-amino acids, either the L-optical isomer or the D-optical isomer can be used. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced. The term “residue” or “amino acid residue” also refers to an amino acid that is incorporated into a protein, polypeptide, or peptide.

Conservative amino acid substitutions are those substitutions that, when made, least interfere with the properties of the original protein, that is, the structure and especially the function of the protein is conserved and is not significantly changed by such substitutions. Examples of conservative amino acid substitutions are known in the art, e.g., as set forth in, for example, U.S. Publication No. 2015/0030628. Conservative substitutions generally maintain (a) the structure of the polypeptide backbone in the area of the substitution, for example, as a sheet or helical conformation; (b) the charge or hydrophobicity of the molecule at the target site; and/or (c) the bulk of the side chain

The substitutions that are generally expected to produce the greatest changes in protein properties are non-conservative, for instance, changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “promoter” is meant an array of nucleic acid control sequences, which direct transcription of a nucleic acid. A promoter includes necessary nucleic acid sequences near the start site of transcription. A promoter also optionally includes distal enhancer or repressor sequence elements. A “constitutive promoter” is a promoter that is continuously active and is not subject to regulation by external signals or molecules. In contrast, the activity of an “inducible promoter” is regulated by an external signal or molecule (for example, a transcription factor). By way of example, a promoter may be a CMV promoter.

As will be appreciated by the skilled practitioner in the art, the term “purified” does not require absolute purity; rather, it is intended as a relative term. Thus, for example, a purified peptide, protein, virus, polynucleotide, or other active compound is one that is isolated in whole or in part from naturally associated proteins and other contaminants. In certain embodiments, the term “substantially purified” refers to a peptide, protein, virus, polynucleotide, or other active compound that has been isolated from a cell, cell culture medium, or other crude preparation and subjected to routine methods, such as, without limitation, fractionation, chromatography, or electrophoresis, to remove various components of the initial preparation, such as proteins, cellular debris, and other components.

A “recombinant” nucleic acid, protein or virus is one that has a sequence that is not naturally occurring or that has a sequence that is made by an artificial combination of two otherwise separated segments of sequence. Such an artificial combination is often accomplished by chemical synthesis or by the artificial manipulation of isolated segments of nucleic acids, for example, by genetic engineering techniques. A “non-naturally occurring” nucleic acid, protein or virus is one that may be made via recombinant technology, artificial manipulation, genetic or molecular biological engineering, or molecular synthesis procedures and techniques, such as those commonly practiced in the art.

By “reduces” is meant a negative alteration (e.g., decrease or reduction) of at least 5%, 10%, 25%, 30%, 40%, 50%, 75%, 80%, 85%, 90%, 95%, 98%, or 100%.

By “reference” is meant a standard or control condition, e.g., a wildtype or nonmutated protein or polynucleotide. In some cases, a reference may be a healthy, uninfected subject or cell, e.g., a subject or cell not infected with influenza virus.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

A compound or antibody that “specifically binds” refers to one that recognizes and binds to a polypeptide, such as a virus polypeptide, peptide, or vaccine product, but which does not substantially recognize and bind to other molecules in a sample, for example, a biological sample, which naturally includes a polypeptide, such as a virus polypeptide or peptide.

Nucleic acid molecules useful in the methods described herein include any nucleic acid molecule that encodes a polypeptide as described, or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pairing to form a double-stranded molecule between complementary polynucleotide sequences (e.g., a gene), or portions thereof, under various conditions of stringency. (See, e.g., Wahl, G. M. and S. L. Berger, (1987), Methods Enzymol., 152:399; Kimmel, A. R., (1987), Methods Enzymol. 152:507).

By way of example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations of these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations of these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, or at least 80% or 85%, or at least or equal to 90%, 93%, 95% or even 99% identical at the amino acid level or nucleic acid to the sequence used for comparison.

“Sequence identity” refers to the similarity between amino acid or nucleic acid sequences that is expressed in terms of the similarity between the sequences. Sequence identity is frequently measured in terms of percentage identity (or similarity or homology); the higher the percentage, the more similar the sequences are. Homologs or variants of a given gene or protein will possess a relatively high degree of sequence identity when aligned using standard methods. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence. In addition, other programs and alignment algorithms are described in, for example, Smith and Waterman, 1981, Adv. Appl. Math. 2:482; Needleman and Wunsch, 1970, J. Mol. Biol. 48:443; Pearson and Lipman, 1988, Proc. Natl. Acad. Sci. U.S.A. 85:2444; Higgins and Sharp, 1988, Gene 73:237-244; Higgins and Sharp, 1989, CABIOS 5:151-153; Corpet et al., 1988, Nucleic Acids Research 16:10881-10890; Pearson and Lipman, 1988, Proc. Natl. Acad. Sci. U.S.A. 85:2444; and Altschul et al., 1994, Nature Genet. 6:119-129. The NCBI Basic Local Alignment Search Tool (BLAST™) (Altschul et al. 1990, J. Mol. Biol. 215:403-410) is readily available from several sources, including the National Center for Biotechnology Information (NCBI, Bethesda, Md.) and on the Internet, for use in connection with the sequence analysis programs blastp, blastn, blastx, tblastn and tblastx.

By “subject” is meant a vertebrate animal, e.g., a mammal, including, but not limited to, a human, a non-human primate, or a non-human animal or mammal, such as a bovine, equine, canine, ovine, or feline mammal, or a sheep, goat, llama, camel, ferret, or a rodent (rat, mouse), gerbil, or hamster. A “subject” may also refer to a non-human animal, or to an avian vertebrate animal. Non-human subjects or non-human animal subjects may also be referred to as “veterinary subjects.” In a nonlimiting example, a subject is one who is infected with a pathogen, such as influenza virus, e.g., an H1 or H3 virus, or who is at risk of infection by such virus, or who is susceptible to such infection. In some aspects as described herein, the subject is a human subject, such as a patient. In some aspects as described herein, the subject is anon-human subject, such as a non-human animal subject or a veterinary subject.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or greater, consecutively, such as to 100 or greater, inclusive of the first and last values and those in between.

As used herein, the terms “treat,” treating,” “treatment,” and the like refer to reducing, diminishing, decreasing, abating, abrogating, alleviating, ameliorating, or eliminating, a disease, condition, disorder, or pathology, and/or symptoms associated therewith. While not intending to be limiting, “treating” typically relates to a therapeutic intervention that occurs after a disease, condition, disorder, or pathology, and/or symptoms associated therewith, have begun to develop so as to reduce the severity of the disease, etc., and the associated signs and symptoms. It will be appreciated that, although not precluded, treating a disorder or condition does not require that the disease, condition, disorder, pathology, or the symptoms associated therewith, be completely eliminated.

As used herein, the terms “prevent,” “preventing,” “prevention,” “prophylactic treatment” and the like, refer to inhibiting or blocking a disease state, or the full or full-blown development of a disease in a subject, or reducing the probability of developing a disease, disorder or condition in a subject, who does not have, but is at risk of developing, or is susceptible to developing, a disease, disorder, or condition.

As referred to herein, a “transformed” cell is a cell into which a nucleic acid molecule or polynucleotide sequence has been introduced by molecular biology techniques. As used herein, the term “transformation” encompasses all techniques by which a nucleic acid molecule or polynucleotide may be introduced into such a cell, including transfection with viral vectors, transformation with plasmid vectors, and introduction of naked nucleic acid (DNA or RNA, e.g., mRNA) by electroporation, lipofection, particle gun acceleration, or other methods known and practiced in the art.

By “vaccine” is meant a preparation of immunogenic material (e.g., protein or nucleic acid), such as a protein or peptide antigen, capable of stimulating (eliciting) an immune response, administered to a subject to treat a disease, condition, or pathology, or to prevent or protect against a disease, condition, or pathology, such as an infectious disease, e.g., a virus infection. The immunogenic material may include, for example, attenuated or killed microorganisms (such as attenuated viruses), or antigenic proteins, peptides, DNA, or RNA derived from such microorganisms. Vaccines may elicit a prophylactic (preventative) immune response in the subject; they may also elicit a therapeutic response immune response in a subject. As mentioned above, methods of vaccine administration vary according to the vaccine, and can include routes or means, such as inoculation (intravenous or subcutaneous injection), ingestion, inhalation, or other forms of administration as known and practiced in the medical art. Inoculations can be delivered by any of a number of routes, including parenteral, such as intravenous, subcutaneous or intramuscular. Vaccines may also be administered with an adjuvant to boost the immune response. Vaccines may be administered to human subjects, non-human subjects, or veterinary subjects. The terms vaccine and immunogen are used interchangeably herein. In an embodiment, a monovalent immunogen/vaccine contains one non-naturally occurring, broadly reactive influenza HA or NA immunogenic polypeptide antigen as described herein, or one HA or NA influenza virus antigen of a certain type or subtype. In an embodiment, a bivalent immunogen/vaccine contains two, same or different, non-naturally occurring, broadly reactive influenza HA or NA immunogenic polypeptide antigens as described herein, and/or two, same or different, HA or NA influenza virus antigens of certain types or subtypes. In an embodiment, a multivalent immunogen/vaccine contains at least two (or more than two) non-naturally occurring, broadly reactive influenza HA or NA immunogenic polypeptide antigens as described herein, and/or at least two HA or NA influenza virus antigens of certain types or subtypes. In an embodiment, a multivalent immunogen/vaccine contains 2, 3, 4, 5, 6, 7, 8, 9, or 10 influenza polypeptide antigens as described and exemplified herein, including a mixture of different influenza immunogenic polypeptide antigens as described and exemplified herein and/or HA or NA influenza virus antigens of certain types or subtypes. In an embodiment, the influenza immunogen/vaccine preparation is bivalent. In an embodiment, the influenza immunogen vaccine preparation is multivalent, e.g., octavalent.

As used herein, a “vector” refers to a nucleic acid (polynucleotide) molecule into which foreign nucleic acid can be inserted without disrupting the ability of the vector to replicate in and/or integrate into a host cell. A vector can include nucleic acid sequences that permit it to replicate in a host cell, such as an origin of replication. An insertional vector can insert itself into a host nucleic acid. A vector can also include one or more selectable marker genes and other genetic elements. An expression vector is a vector that contains the necessary regulatory sequences to allow transcription and translation of inserted gene or genes in a host cell. In some embodiments of the present disclosure, the vector encodes an influenza HA, NA or M1 protein. In some embodiments, the vector is the pTR600 expression vector (U.S. Patent Application Publication No. 2002/0106798; Ross et al., 2000, Nat Immunol. 1(2):102-103; and Green et al., 2001, Vaccine 20:242-248).

By “virus-like particle (VLP)” is meant virus particles made up of one of more viral structural proteins, but lacking the viral genome. Because VLPs lack a viral genome, they are non-infectious and yield safer and potentially more-economical vaccines and vaccine products. In addition, VLPs can often be produced by heterologous expression and can be easily purified. Most VLPs comprise at least a viral core protein that drives budding and release of particles from a host cell. One example of such a core protein is influenza M1. In some embodiments herein, an influenza VLP comprises the HA, NA and M1 proteins. In some cases, influenza VLPs can be produced by transfection of host cells with plasmids encoding the HA, NA and M1 proteins. After incubation of the transfected cells for an appropriate time to allow for protein expression (such as for approximately 72 hours), VLPs can be isolated from cell culture supernatants. By way of example, a protocol for purifying or isolating influenza VLPs from cell supernatants involves low speed centrifugation (to remove cell debris), vacuum filtration and ultracentrifugation of the VLPs through 20% glycerol. A virus-like particle may also include a subviral particle (SVP), which is typically smaller in size than a virus and constitutes a particle without a virus capsid or genome.

Unless specifically stated or obvious from context, as used herein, the term “or” is understood to be inclusive. Unless specifically stated or obvious from context, as used herein, the terms “a”, “an”, and “the” are understood to be singular or plural. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description.

Unless specifically stated or obvious from context, as used herein, the term “about” is understood as within a range of normal tolerance in the art, for example within 2 standard deviations of the mean. About may be understood as being within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 presents an illustration of the timeline of a study performed using pre-immune and naïve ferrets immunized (vaccinated) with a combination of broadly-reactive, influenza HA polypeptide immunogens as described herein (Example 2) to evaluate the efficacy of the polypeptides as immunogens (e.g., a vaccine) to protect against and/or reduce the effects of virus challenge and severe disease following immunization. As shown in the timeline, 60 days prior to immunization/vaccination (day−60) of the animals with the broadly-reactive, influenza HA polypeptide immunogens, a group of animals was made pre-immune by infecting them with the influenza virus strains A/California/2009 (H1N1), A/Panama/1999 (H3N2) and B/Hong Kong/2001 (IBV, Influenza B virus). These strains are found historically in the normal human population and mimic in the animals a typical viral load or component of viruses that would be found in a normal, pre-immune human population prior to immunizing/vaccinating the animals with the broadly reactive HA polypeptide immunogens described herein. On day −30, the animals were bled to confirm seroconversion. On days 0 and 28 of the study, the ferrets were bled and immunized/vaccinated with the broadly reactive HA polypeptide immunogens and c-di-AMP adjuvant (15 μg per antigen+50 μg c-di-AMP). as described herein (Example 2). Four weeks after the second vaccination, the animals were challenged with influenza viruses A/Brisbane/02/2018 (H1N1), B/Washington/02/2019 (IBV), or A/Vietnam/1203/2004 (H5N1) (day 56 of the study). Nasal washes were carried out on days 1, 3, 5 and 7 post-infection, between the time of challenge (day 56) and the end of the study (day 60). During this period, clinical signs and weights of the animals were monitored daily.

FIGS. 2A-2D present graphs showing the results of ELISA analyses performed on day 56 of the study described in Example 2 and illustrated in FIG. 1 using sera obtained after blood was obtained from the immunized ferrets. The sera were tested for the presence of total IgG antibody response in the ferrets after the second vaccination. The amount of antibody binding to different immunogenic HA polypeptide antigens coated on the microtiter plates was assessed. Ferrets were vaccinated intranasally twice at four-week intervals with c-di-AMP as adjuvant. The immunized/vaccinated animal groups represented are: pre-immune ferrets administered octavalent Cobra vaccine (FIG. 2A); pre-immune ferrets administered mock vaccine (FIG. 2B); naïve ferrets administered octavalent Cobra vaccine (FIG. 2C), naïve ferrets administered mock vaccine (FIG. 2D). Total IgG antibody titers were determined against each of the 8 HA immunogenic polypeptide antigens (Cobra antigens), as indicated on the x-axis. The y-axis indicates OD 414 nm values. Each dot represents an individual ferret. For each independent experiment, sera were assayed in duplicate. The results demonstrated that immunization with the octavalent HA immunogenic polypeptide antigens (Cobra antigens) as described herein elicited an immune response and the production of antibodies that bound to all components of the HA immunogenic polypeptides/vaccine (FIGS. 2A and 2C). Antibodies from the pre-immune mock animals bound to the H1, N1 and N2 HA antigens (FIG. 2B). Antibodies from the naive mock animals showed no binding to the HA antigens (FIG. 2D). An OD value of 1 was chosen as a minimum due to non-specific binding to the his-tag present on the polypeptides.

FIGS. 3A and 3B present graphs of the results of ELISA analyses performed to compare the total IgG antibody response before and after vaccination in pre-immune groups of ferrets on day 0 and day 56 of the study described in FIG. 1 using sera obtained from pre-immune ferrets immunized with HA immunogenic polypeptide antigens or from pre-immune, mock-immunized ferrets. Sera were collected before immunization/vaccination (day 0 (d0)) and after final immunization/vaccination (day 56 (d56)) for individual pre-immune ferrets that were immunized with HA polypeptide immunogen (Cobra) (FIG. 3A) and pre-immune ferrets given mock vaccination (FIG. 3B). The results demonstrated that pre-immune ferrets that were immunized with HA immunogenic polypeptide antigens as described herein had an increase in antibodies to all HA components of the immunogenic polypeptide antigens/vaccine, with statistically significant increases in antibodies generated against the Z1, NG3, IAN8, Q6, and BC2 immunogens. One-way ANOVA was used to analyze the statistical differences between d0 and d56 ELISA results (FIGS. 2A-2D) for each group by GraphPad Prism 9 software (GraphPad, San Diego, CA, USA). A p value less than 0.05 was defined as statistically significant (*, p<0.05; **, p<0.01; ***, p<0.001; ****, p<0.0001).

FIGS. 4A and 4B present graphs showing the results of ferret serum HAI antibody titers against influenza H1N1 viruses before and after vaccination. Ferrets were vaccinated intranasally twice at four-week intervals with c-di-AMP as adjuvant. Sera were collected before vaccination (FIG. 4A) and 4 weeks after second vaccination (FIG. 4B) for performing HAI assays against a panel of 6 H1N1 influenza viruses. The viruses listed on the x-axis are: A/Solomon Islands/03/2006 (SI/06) A/Brisbane/59/2007 (Bris/07), A/California/07/2009 (CA/09), A/Michigan/45/2015 (Mich/15), A/Brisbane/02/2018 (Bris/18), and A/Guangdong-Maonan/SWL1536/2019 (GD/19). The y-axis indicates the log 2 HAI titers for each vaccinated group and presents them as absolute mean values ±SEM. The dotted lines indicate HAI titers ranging from 1:40 (lower line) and 1:80 (upper line). Statistical differences between day 0 and day 56 HAI titers for each vaccine group were analyzed using nonparametric one-way ANOVA by GraphPad Prism 9 software (GraphPad, San Diego, CA, USA). A p value of less than 0.05 was defined as statistically significant (*, p<0.05; **, p<0.01; ***, p<0.001; ****, p<0.0001). The results showed that the pre-immune mock animal groups had statistically significant lower HAI titers for Mich/15 and Bris/18. The pre-immune animals immunized with HA immunogenic polypeptides (COBRA antigens) maintained high HAI titers, and the naïve animal groups immunized with HA immunogenic polypeptides (COBRA antigens) reached HAI titers of 1:40 for pandemic-like virus strains, except Guangdong/19.

FIGS. 5A and 5B present graphs showing the results of ferret serum HAI antibody titers against influenza H3N2 viruses before and after vaccination. Ferrets were vaccinated intranasally twice at four-week intervals with c-di-AMP as adjuvant. The vaccine groups were: pre-immune ferrets given octavalent recombinant influenza immunogenic polypeptides (Cobra antigens), (black bar); naïve ferrets given octavalent recombinant influenza immunogenic polypeptides (Cobra antigens), (white bar); or pre-immune ferrets given mock vaccination (grey bar). Sera were collected before vaccination (FIG. 5A) and at 4 weeks after the second vaccination (FIG. 5B) for HAI assay against a panel of 6 H3N2 influenza viruses. The viruses listed on the x-axis are: A/Switzerland/9715293/2013 (Switz/13), A/Hong Kong/4801/2014 (HK/14), A/Singapore/IFNIMH-16-0019/2016 (Sing/16), A/Kansas/14/2017 (KS/17), A/South Australia/34/2019 (SA/19), and A/Hong Kong/2671/2019 (HK/19). The y-axis indicates the log 2 HAI titers for each vaccinated group and presents them as absolute mean values ±SEM. The dotted lines indicate HAI titers ranging from 1:40 (lower line) and 1:80 (upper line). Statistical differences between day 0 and day 56 HAI titers for each vaccine animal group were analyzed using nonparametric one-way ANOVA by GraphPad Prism 9 software (GraphPad, San Diego, CA, USA). A p value of less than 0.05 was defined as statistically significant (*, p<0.05; **, p<0.01; ***, p<0.001; ****, p<0.0001).

FIGS. 6A and 6B present graphs showing the results of ferret serum HAI antibody titers for Influenza B viruses (IBV) before and after vaccination. Ferrets were vaccinated twice intranasally at four-week intervals with c-di-AMP as adjuvant. Vaccine groups were: pre-immune ferrets given octavalent recombinant influenza immunogenic polypeptides (Cobra antigens), (black bar), naïve ferrets given octavalent recombinant influenza immunogenic polypeptides (Cobra antigens), (white bar), or pre-immune ferrets given mock vaccination (grey bar). Sera were collected before vaccination (FIG. 6A) and 4 weeks after the second vaccination (FIG. 6B) for HAI assay against a panel of 6 IBV influenza viruses. The viruses listed on the x-axis are: for Yamagata-like lineages B/Florida/04/2006 (B/FL/06), B/Massachusetts/02/2012 (B/Mass/12), B/Phuket/3073/2013 (B/Phuk/13); for Victoria-like lineages B/Brisbane/60/2008 (B/Bris/08), B/Colordado/06/2017 (B/CO/17), and B/Washington/02/2019 (B/WA/19). The y-axis indicates the log 2 HAI titers for each vaccinated group and presents them as absolute mean values ±SEM. The dotted lines indicate HAI titers ranging from 1:40 (lower line) and 1:80 (upper line). Statistical differences between day 0 and day 56 HAI titers for each vaccine group were analyzed using nonparametric one-way ANOVA by GraphPad Prism 9 software (GraphPad, San Diego, CA, USA). A p value of less than 0.05 was defined as statistically significant (*, p<0.05; **, p<0.01; ***, p<0.001; ****, p<0.0001).

FIGS. 7A and 7B demonstrate serum HAI antibody titers in study ferrets against influenza H5 viruses before and after vaccination. Ferrets were vaccinated intranasally twice at four-week intervals with c-di-AMP as adjuvant. Vaccine groups were: pre-immune ferrets given octavalent recombinant influenza immunogenic polypeptides (Cobra antigens), (black bar); naïve ferrets given octavalent recombinant influenza immunogenic polypeptides (Cobra antigens), (white bar); or pre-immune ferrets given mock vaccination (grey bar). Sera were collected before vaccination (FIG. 7A) and at 4 weeks after the second vaccination (FIG. 7B) for HAI assay against a panel of 6 H5 influenza viruses: A/Vietnam/1203/2004 (H5N1, Vn/04), A/whooper swan/Mongolia/244/2005 (H5N1, ws/Mo/05) A/Egypt/321/2007 (H5N1, Eg/07), A/Hubei/01/2010 (H5N1, Hu/10), A/Guizhou/01/2013 (H5N1, Gu/13). A/Sichuan/26221/2014 (H5N6, Si/14). The y-axis indicates the log 2 HAI titers for each vaccinated group and presents them as absolute mean values ±SEM. The dotted lines indicate HAI titers ranging from 1:40 (lower line) and 1:80 (upper line). Statistical differences between day 0 and day 56 HAI titers for each vaccine group were analyzed using nonparametric one-way ANOVA by GraphPad Prism 9 software (GraphPad, San Diego, CA, USA). A p value of less than 0.05 was defined as statistically significant (*, p<0.05; **, p<0.01; ***, p<0.001; ****, p<0.0001).

FIGS. 8A and 8B show body weight and survival curves of ferrets after challenge with influenza H5N1 virus. Ferrets were vaccinated twice intranasally at four-week intervals with c-di-AMP as adjuvant. Vaccine groups were pre-immune ferrets immunized with octavalent recombinant influenza immunogenic polypeptides (Cobra antigens), (black line); naïve ferrets immunized with octavalent recombinant influenza immunogenic polypeptides (Cobra antigens), (black line with circle); or pre-immune, mock-immunized ferrets (grey line). Four weeks after the final immunization/vaccination, ferrets were intranasally infected with a lethal dose of virus A/Vietnam/1203/2004 (10⁵ PFU) in a volume of 1 mL. The animals were observed for clinical signs and their body weights were recorded daily post infection (FIG. 8A). Survival curve data after infection indicate that all of the animals survived the lethal virus challenge (FIG. 8B).

FIGS. 9A and 9B show body weight curves of ferrets after challenges with influenza virus. Ferrets were vaccinated twice intranasally at four-week intervals with c-di-AMP as adjuvant. Vaccine groups were: pre-immune ferrets immunized with octavalent recombinant influenza immunogenic polypeptides (Cobra antigens), (black line); naïve ferrets immunized with octavalent recombinant influenza immunogenic polypeptides (Cobra antigens), (black line with circle); pre-immune ferrets given mock vaccination (grey line); or naïve ferrets given mock vaccination (dashed black line). Four weeks after the final vaccination, the ferrets were intranasally infected with influenza virus A/Brisbane/02/2018 (10⁸ PFU), (FIG. 9A), or with influenza virus (b) B/Washington/02/2019 (10⁷ PFU), (FIG. 9B), in a volume of 1 mL. The animals were observed for clinical signs and their body weights were recorded daily post infection. Statistical differences of body weight losses are shown in the tables in FIGS. 10A and 10B.

FIGS. 10A and 10B present tables showing the statistical differences of body weight loss between the groups of immunized ferrets challenged with different virus strains (H1N1 versus IBV). The body weight loss values from ferrets challenged with H1N1 A/Brisbane/02/2018 (FIG. 10A) or IBV B/Washington/02/2019 (FIG. 10B) as described in FIGS. 9A and 9B above were analyzed for statistical differences for each day of infection via two-way ANOVA with multiple comparisons by GraphPad Prism 9 software (GraphPad, San Diego, CA, USA). A p value of less than 0.05 was defined as statistically significant (*, p<0.05; **, p<0.01; ***, p<0.001; ****, p<0.0001; n.s., not significant).

FIGS. 11A-11C show graphs of viral titers in upper respiratory tracts of ferrets after infection with influenza virus A/Brisbane/02/2018 (H1N1). Ferrets were vaccinated intranasally twice at four-week intervals with c-di-AMP as adjuvant. Vaccine groups are indicated on the x-axis. Four weeks after the second vaccination, the groups of animals were challenged with H1N1 A/Brisbane/02/2018 virus. Nasal washes were taken from the animals on day 1 (FIG. 11A), day 3 (FIG. 11B), and day 5 (FIG. 11C) post infection and viral titers were determined. Viral titers in the nasal washes are presented as PFU/mL shown on the y-axis. Each dot represents an individual ferret.

FIGS. 12A-12C show graphs of nasal wash titers of ferrets after infection with influenza virus B/Washington/02/2019 (IBV). Ferrets were vaccinated intranasally twice at four-week intervals with c-di-AMP as adjuvant. Vaccine groups are indicated on the x-axis. Four weeks after second vaccination, the groups of animals were challenged with IBV B/Washington/02/2019 virus. Nasal washes were taken on day 1 (FIG. 12A), day 3 (FIG. 12B), and day 5 (FIG. 12C) post infection and viral titers were determined. Viral titers in nasal washes are presented as PFU/mL shown on the y-axis. Each dot represents an individual ferret.

FIGS. 13A-13N show graphs of the results of a hemagglutination inhibition (HAT) assay performed on sera from 75 influenza-naive BALB/c mice (n=5/group) on day 70 post initial vaccination. Serum collected from 75 influenza-naive BALB/c mice (n=5/group) on day 70 post initial vaccination were assessed for HAI activity against a panel of historical H3N2 vaccine strains isolated during 2012 to 2019 (x axis). The Log₂ HAI titers are reported as absolute mean values 6 standard error of the mean (SEM) (y axis). The lower dotted line represents an HAI titer of 1:40, and the upper dotted line represents an HAI titer of 1:80. The mice were vaccinated with 3 mg recombinant influenza hemagglutinin (rHA) immunogenic poly peptide antigens as follows: Mock (FIG. 13A), J1 (FIG. 13B). J2 (FIG. 13C). J3 (FIG. 13D), J4 (FIG. 13E), NG1 (FIG. 13F), NG2 (FIG. 13G), NG3 (FIG. 13H), Switz/13 (FIG. 13I), HK/14 (FIG. 13J), Sing/16 (FIG. 13K), Kan/17 (FIG. 13L), Switz/17 (FIG. 13M), and SA/19 (FIG. 13N), HAI titers were statistically analyzed using nonparametric one-way analysis of variance (ANOVA) by Prism 9 software (GraphPad Software, Inc., San Diego, CA). A P value of less than 0.05 was defined as statistically significant (*, P, 0.05; **, P, 0.01; ***, P, 0.001; ****, P, 0.0001). The H3N2 viruses belong to the following clades: Tx/12 (3c2), Switz/13 (3c3.a), HK/14 (3c.2a), Sing/16 (3c2.al), Kan/17 (3c3.a), Tx17 (3c3.a), Switz/17 (3c3.a2), SA/19 (3c2.ab/131K), and HK/19 (3c2.alb/137F).

FIG. 14 presents a graph showing day 89 lung viral titers. Lungs were collected from n=3 nice per group on day 89 (3 days post A/Kansas/14/2017 challenge) to assess the viral load present in the lung tissue. Vaccine groups are listed on the x axis, and PFU/g of lung tissue values are reported on the y axis. A nonparametric one-way ANOVA was used to analyze statistical differences between groups using Prism 9 software (GraphPad Software, Inc., San Diego, CA). A P value of less than 0.05 was defined as statistically significant (*, P,0.05; **, P ,0.01; ***, P, 0.001, ****, P,0.0001).

FIGS. 15A-15F present graphs showing the results of focus reduction assay (FRA) against an H3N2 influenza virus panel on day 72 carried out using serum from preimmune mice to assess the presence of antibodies directed against the H3N2 virus panel. Serum collected from 136 H1+H3 preimmune DBA/2J mice (n=8/group) that were vaccinated with monovalent and bivalent formulations of H1+H3 recombinant influenza immunogenic polypeptides (Cobra antigens) or WT rHA on day 72 post initial infection were pooled for each group and assessed for FRA neutralization against a panel of historical H3N2 vaccine strains from 2016-2019. Sera from mice vaccinated with monovalent antigens were tested against the following H1N1 viruses: A/Singapore/IFNIMH-16-0019/2016 (FIG. 15A), A/Kansas/14/2017 (FIG. 15B), and A/Hong Kong/2671/2019 (FIG. 15C). Sera from mice vaccinated with cocktails of bivalent H1+H3 antigens were tested against the following H1N1 viruses: A/Singapore/IFNIMH-16-0019/2016 (FIG. 15D), A/Kansas/14/2017 (FIG. 15E), and A/Hong Kong/2671/2019 (FIG. 15F). The lower dotted line represents 80% neutralization (Neut80); the middle dotted line represents 50% neutralization (Neut50; and the upper dotted line represents no neutralization of viral infection.

FIGS. 16A-16F present graphs showing the results of focus reduction assays (FRA) against an H1N1 influenza virus panel carried out using serum from preimmune mice on day 72. On day 72 post initial infection, serum collected from 136 H1+H3 preimmune DBA/2J mice (n=8/group) that were vaccinated with monovalent and bivalent formulations of H1+H3 recombinant influenza immunogenic polypeptides (Cobra antigens) or WT rHA were pooled for each group and assessed for FRA neutralization against a panel of historical H1N1 vaccine strains from 2009 to 2019. Sera from mice vaccinated with monovalent antigens were tested against the following H1N1 viruses: A/California/07/2009 (FIG. 16A), A/Brisbane/2/2018 (FIG. 16B), and A/Guangdong Maonan/SWL1536/2019 (FIG. 16C). Sera from mice vaccinated with cocktails of bivalent H1+H3 antigens were tested against the following H1N1 viruses: A/California/07/2009 (FIG. 16D), A/Brisbane/2/2018 (FIG. 16E), and A/Guangdong Maonan/SWL1536/2019 (FIG. 16F). The lower dotted line represents 80% neutralization (Neut80); the middle dotted line represents 50% neutralization (Neut50); and the upper dotted line represents no neutralization of viral infection.

FIG. 17 presents an illustration of the timeline of a study performed using mice immunized (vaccinated) with broadly-reactive, influenza HA polypeptide immunogens as described herein (Example 2) to evaluate the efficacy of the polypeptides as immunogens (e.g., a vaccine) to protect against and/or reduce the effects of virus challenge and severe disease following immunization. In the animal study, eighty-eight BALB/c mice (n=11) were intramuscularly immunized/vaccinated with the broadly-reactive, influenza HA immunogens or wild-type HA in virus-like particle (VLP) immunogens/vaccines formulated with ADDAVAX™ adjuvant at weeks 0, 4, and 8. At weeks 6 and 10 post vaccination, blood was collected from the animals, and serum was separated for analysis. At week 12, all mice were intranasally inoculated with a challenge of 5×10⁴ PFU of A/California/07/2009 H1N1 virus. Lung tissues (n=3/group) were harvested on days 3 and 6 post infection (3 dpi and 6 dpi) and were evaluated for histopathology and virus titration.

FIGS. 18A-18D present graphs demonstrating body weight and survival curves after influenza virus infection of the mice immunized/vaccinated with broadly-reactive, recombinant influenza HA immunogens as described herein or with wildtype rHA proteins versus PBS control following the protocol shown in FIG. 17. FIG. 18A shows percent of original body weight loss of mice post infection. Mice were observed for clinical signs for 14 days and their body weights were recorded daily post infection. The dotted line indicates 80% of body weights on day 0 post infection. FIG. 18B shows survival curves after infection with A/California/07/2009 virus. Another group of sixty-four (64) DBA/2J mice were intramuscularly immunized/vaccinated with broadly-reactive, recombinant influenza HA immunogens or wild-type rHA immunogens/vaccines formulated with ADDAVAX™ adjuvant using the above-described immunization/vaccination regimen. At week 12, all of the mice were intranasally infected with 8.75×10⁶ PFU of A/Brisbane/02/2018 H1N1 virus as virus challenge. FIG. 18C shows body weight loss curves of DBA/2J mice after infection (challenge) with A/Brisbane/02/2018 H1N1 virus. FIG. 18D shows survival curves after infection (challenge) with A/Brisbane/02/2018 virus.

FIGS. 19A-19E present graphs showing serum HAI antibody titers in mice post immunization/vaccination against a panel of H1N1 viruses. Immunologically naïve BALB/c mice were immunized/vaccinated three times at 4-week intervals with VLP immunogen/vaccine containing polynucleotides encoding broadly-reactive, recombinant influenza H1N1 HA immunogen Y2 (e.g., SEQ ID NO: 15) or with H1N1 wildtype Bris/07, CA/09, or Bris/18 VLP immunogens/vaccines. Sera were collected from the animals on week 10 post first-immunization/vaccination, and an HAI assay using the sear was performed against a panel of 7 H1N1 influenza viruses: broadly-reactive, recombinant influenza H1 HA immunogen (FIG. 19A); Bris/18 (FIG. 19B); CA/09 (FIG. 19C); Bris/07 (FIG. 19D); PBS (FIG. 19E). The y axis indicates the log 2 HAI titers for each immunized/vaccinated group of animals and presents them as absolute mean values ±SEM. The dotted lines indicate HAI titers ranging from 1:40 (lower line) and 1:80 (upper line). HAI titers were statistically analyzed using nonparametric one-way ANOVA by GraphPad Prism 9 software (GraphPad, San Diego, CA, USA). A p value of less than 0.05 was defined as statistically significant (*, p<0.05; **, p<0.01; ***, p<0.001; ****, p<0.0001).

FIGS. 20A and 20B present graphs showing neutralizing antibody titers in mouse sera post vaccination. Immunologically naive BALB/c mice (n=11/group) were vaccinated three times at 4-week intervals with a broadly reactive, HA immunogenic polypeptide (Y2 COBRA H1N1) VLP vaccine, or with H1N1 wild-type Bris/07, CA/09, or Bris/18 VLP vaccines. At week 10 post vaccination, sera were collected and used to carry out a FRA assay against A/California/07/2009 (FIG. 20A) and A/Brisbane/02/2018 (FIG. 20B) viruses. For each virus, the virus concentration was standardized to 1.2×10⁴ FFU/mL, and the virus alone infected well was standardized as 100% infection. The x-axis indicates log 2 sera dilution, and the y-axis represents the percentage of infected cells compared to virus-only infected control wells. The dotted lines represent the 50% inhibition (upper line) and the 80% inhibition (lower line) activity of antibodies present in the antisera.

FIGS. 21A-21C present graphs showing total IgG antibody responses in mice. Vaccine responses in BALB/c mice were evaluated at week 10 post vaccination with a broadly reactive, HA immunogenic polypeptide vaccine (H1 Cobra HA), wild-type HA VLP vaccines (e.g., Bris/18 HA, CA/09 HA, Bris/07 HA), or PBS (x-axis) formulated with ADDAVAX™ adjuvant. IgG antibody titers were determined against A/California/07/2009 HA protein (FIG. 21A), A/Brisbane/02/2018 HA protein (FIG. 21B), or cH6/1 HA protein (Chimeric rHA with globular head from A/California/07/2009 HA and stalk form subtype H6 influenza virus HA), (FIG. 21C). The data are presented as area under curve (AUC) obtained OD141 values from 3-fold serially diluted sera plus SEM. For each independent experiment, mouse sera were assayed in duplicate. One-way ANOVA was used to analyze the statistical differences between groups by GraphPad Prism 9 software (GraphPad, San Diego, CA, USA). A p value less than 0.05 was defined as statistically significant (*, p<0.05; **, p<0.01; ***, p<0.001; ****, p<0.0001).

FIGS. 22A and 22B present graphs showing viral titers in the lung tissues of BALB/c and DBA/2J mice. BALB/c mice were intramuscularly vaccinated with a broadly reactive, HA immunogenic polypeptide (H1 Cobra HA) or with wild-type HA VLP vaccines (e.g., Bris/18 HA, CA/09 HA, Bris/07 HA), (x-axis), and then challenged with H1N1 A/California/07/2009 virus at week 12 post vaccination (FIG. 22A). Lung samples (n=3/group) were collected at day 3 post infection, and lung viral titers were determined. Another set of DBA/2J mice that had been immunized/vaccinated with the same vaccines as noted above and delivered in a rHA format were challenged with A/Brisbane/02/2018 at 12 weeks post vaccination (FIG. 22B). Lung samples (n=3) were harvested on day 3 post infection, and lung viral titers were determined. Viral titers in lung tissue are presented as PFU/mL shown on the y-axis. The x-axis indicates the different vaccines used in the study. A nonparametric one-way ANOVA was used to analyze statistical differences between groups using GraphPad Prism 9 software. A p value less than 0.05 was defined as statistically significant (*, p<0.05; **, p<0.01; ***, p<0.001; ****, p<0.0001).

### DETAILED DESCRIPTION OF THE DISCLOSURE

Featured herein are synthetic (non-naturally occurring), immunogenic antigens, e.g., protein (polypeptides) and glycoprotein antigens, derived from the influenza (“flu”) hemagglutinin (HA) protein of influenza virus strains, e.g., H1 or H3, that elicit a potent, broadly reactive and long-lasting immune response in a subject. In an embodiment, the subject is a mammalian subject. In an embodiment, the subject is a human subject. In an embodiment, the subject is an avian subject. In an embodiment, the HA protein is a full length HA polypeptide or a soluble HA (sHA) polypeptide. In an embodiment, a sHA polypeptide does not include a transmembrane (TM) or a tail domain. Such immunogenic antigens are also referred to as immunogens, immunogenic polypeptides, proteins, or peptides, or vaccines herein.

Provided are broadly reactive immunogens that protect against disease caused by the influenza strains, such as H1 or H3, e.g., H1N1 or H3N2. In an embodiment, fully synthetic protein antigens are featured, such as influenza virus HA protein antigens. Such HA antigens are synthetic proteins not found in nature, yet they retain all of the functions of a natural influenza virus HA protein and are immunogenic, i.e., they can elicit an immune response, in particular, a broadly active immune response in the form of neutralizing antibodies and/or reactive T lymphocytes, following administration or delivery to, or introduction into, a subject, especially for influenza virus antigen immunogens. In embodiments, the HA protein antigen is a full length HA polypeptide or a soluble HA (sHA) polypeptide. Also provided are immunogenic compositions, e.g., vaccines, comprising the synthetic virus protein antigens, or nucleic acids (e.g., DNA or RNA) encoding the antigens.

An HA amino acid sequence and a protein antigen having such sequence are particularly for use as an immunogen, or in an immunogenic composition, e.g., a vaccine, that elicits a broadly reactive immune response in a subject, particularly a human subject, to whom the composition, or vaccine, is administered. The synthetic antigens are designed to generate a broadly active immune response, particularly in the form of neutralizing antibodies, along with a cellular immune response in some cases, in a subject. In an embodiment, the subject is a mammalian subject, in particular, a human subject. Such antigens are beneficial as immunogens, which elicit an immune response (e.g., production of neutralizing antibodies and/or a cellular immune response) against the virus, in particular, in cases in which more than one strain of virus co-circulate at a given time. By way of example, the broadly reactive influenza immunogenic antigens can be derived from influenza virus that frequently mutates parts of its genome to escape immune pressure, and as a consequence, evades immune surveillance in a subject whose immune system is not primed or stimulated to generate antibodies against antigenic epitopes (determinants) on the virus antigens following infection. Thus, the synthetic influenza virus antigens, e.g., H1 or H3 HA antigen, comprise amino acid (or polynucleotide) sequences that will elicit greater numbers of neutralizing antibodies (and/or an improved cellular immune response) against potential influenza virus variants exhibiting antigenic drift compared with wild-type antigen sequences.

An HA immunogenic protein, or immunogen, of H1 or H3 influenza viruses, as described herein can be employed in an immunogenic composition or as a vaccine that may afford protection against many virus strains over time. In embodiments, the HA immunogenic protein or immunogen of H1 or H3 influenza viruses comprises a sequence as set forth in SEQ ID NOS: 1-17 herein. The broadly reactive virus antigen immunogens and vaccines described herein are advantageous in that they are designed to provide broader and longer-lasting protection against several different viral (e.g., influenza virus) strains (or clades), such as those arising in different areas.

The immunogenic influenza virus HA antigens, including full length and soluble forms of HA, described herein may be used in immunogenic compositions (e.g., vaccines) that can afford protective immunity against influenza virus infection and disease in a subject. The protective immunity is provided in the subject through the elicitation of broadly reactive, anti-HA specific antibody or cellular immune responses that protect the subject against virus strains that may have mutated or experienced antigenic drift.

### Influenza Virus

Influenza viruses are segmented negative-strand RNA viruses that belong to the Orthomyxoviridae family. There are three types of Influenza viruses: types A, B and C. Influenza A viruses infect a wide variety of birds and mammals, including humans, horses, marine mammals, pigs, ferrets, and chickens. In animals, most influenza A viruses cause mild localized infections of the respiratory and intestinal tract. However, highly pathogenic influenza A strains, such as, by way of nonlimiting example, the H1N1 (“H1”), H3N2 (“H3”), or H5N1 (“H5”), or H7, or H9 strains, cause systemic infections in poultry in which mortality may reach 100%. Animals infected with influenza A often act as a reservoir for the influenza viruses and certain subtypes have been shown to cross the species barrier to humans in whom they can cause severe disease and devastating flu outbreaks that can lead to death of the infected human subjects.

Influenza A viruses can be classified into subtypes based on allelic variations in antigenic regions of two genes that encode surface glycoproteins, namely, hemagglutinin (HA) and neuraminidase (NA) which are required for viral attachment and cellular release, respectively. Currently, sixteen subtypes of HA (H1-H16) and nine NA (N1-N9) antigenic variants are known for influenza A virus. Previously, only three subtypes were known to circulate in humans (H1N1 or H1N2). However, in recent years, for example, the pathogenic H5N1 subtype of avian influenza A has been reported to cross the species barrier and infect humans as documented in Hong Kong in 1997 and 2003, leading to the death of several patients.

In humans, the avian influenza virus infects cells of the respiratory tract as well as the intestinal tract, liver, spleen, kidneys and other organs. Symptoms of avian influenza infection include fever, respiratory difficulties, including shortness of breath and cough, lymphopenia, diarrhea and difficulties regulating blood sugar levels. In contrast to seasonal influenza, the group most at risk is healthy adults which make up the bulk of the population. Due to the high pathogenicity of certain avian influenza A subtypes and their demonstrated ability to cross over to infect humans, there is a significant economic and public health risk associated with these viral strains, including a real epidemic and pandemic threat.

The influenza A virus genome encodes nine structural proteins and one nonstructural (NS1) protein with regulatory functions. The influenza virus segmented genome contains eight negative-sense RNA (nsRNA) gene segments (PB2, PB1, PA, NP, M, NS, HA and NA) that encode at least ten polypeptides, including RNA-directed RNA polymerase proteins (PB2, PB 1 and PA), nucleoprotein (NP), neuraminidase (NA), hemagglutinin, e.g., subunits HAT, frequently referred to as the “head” subunit; and HA2, frequently referred to as the “tail” or “stalk” subunit; the matrix proteins (M1 and M2); and the non-structural proteins (NS1 and NS2) (See, e.g., Krug et al., 1989, In: The Influenza Viruses, R. M. Krug, ed., Plenum Press, N.Y., pp. 89 152).

The ability of influenza virus to cause widespread disease is due to its ability to evade the immune system by undergoing antigenic change, which is believed to occur when a host is infected simultaneously with both an animal influenza virus and a human influenza virus. During mutation and reassortment in the host, the virus may incorporate an HA and/or NA surface protein gene from another virus into its genome, thereby producing a new influenza subtype and evading the immune system.

Because of antigenic variation (drift) in the circulating strains of influenza viruses, in particular, in the HA and NA proteins of the virus, the efficacy of immunogenic compositions, e.g., vaccines, against influenza virus has frequently been less than optimal and sub-par. The immunogens, compositions and methods described herein provide broadly reactive HA antigens that generate a broadly reactive immune response, particularly, in the form of neutralizing antibodies that bind to the viral antigens and neutralize the activity of the virus (e.g., its ability to infect cells), to treat influenza and its symptoms more effectively.

### Influenza Virus Hemagglutinin (HA) and Neuraminidase (NA) Proteins

HA is a viral surface glycoprotein that generally comprises approximately 560 amino acids (e.g., 566 amino acids) and represents 25% of the total virus protein. As described herein, HA is a protein antigen that is highly useful as an immunogen because it contains a diverse repertoire of epitopes against which antibodies are generated in a subject or host that encounters the HA antigen of influenza viruses during infection.

HA is responsible for adhesion of the viral particle to, and its penetration into, a host cell, particularly, in the respiratory epithelium, in the early stages of infection. Cleavage of the virus HA0 precursor into the HA1 and HA2 sub-fragments is a necessary step for the virus to infect a cell. Thus, cleavage is required to convert new virus particles in a host cell into virions capable of infecting new cells. Cleavage is known to occur during transport of the integral HA0 membrane protein from the endoplasmic reticulum of the infected cell to the plasma membrane. During transport, HA undergoes a series of co- and post-translational modifications, including proteolytic cleavage of the precursor HA into the amino-terminal fragment HAT (“head”) and the carboxy terminal HA2 (“tail” or “stalk”). One of the primary difficulties in growing influenza strains in primary tissue culture or established cell lines arises from the requirement for proteolytic cleavage activation of the influenza hemagglutinin in the host cell.

Although it is known that an uncleaved HA can mediate attachment of the virus to its neuraminic acid-containing receptors on a cell surface, it is not capable of the next step in the infectious cycle, which is fusion. It has been reported that exposure of the hydrophobic amino terminus of HA2 by cleavage is required so that it can be inserted into the target cell, thereby forming a bridge between the virus and the target cell membranes. This process is followed by fusion of the two membranes and entry of the virus into the target cell.

Proteolytic activation of HA involves cleavage at an arginine residue by a trypsin-like endoprotease, which is often an intracellular enzyme that is calcium-dependent and has a neutral pH optimum. Since the activating proteases are cellular enzymes, the infected cell type determines whether the HA is cleaved. The HA of the mammalian influenza viruses and the nonpathogenic avian influenza viruses are susceptible to proteolytic cleavage only in a restricted number of cell types. On the other hand, by way of example, HA of pathogenic avian viruses among the H5 and H7 subtypes are cleaved by proteases present in a broad range of different host cells. Thus, there are differences in host range resulting from differences in hemagglutinin cleavability which are correlated with the pathogenic properties of the virus.

Neuraminidase (NA) is a second membrane glycoprotein of the influenza viruses. The presence of viral NA has been shown to be important for generating a multi-faceted protective immune response against an infecting virus. For most influenza A viruses, NA is 413 amino acids in length and is encoded by a gene of 1413 nucleotides. Nine different NA subtypes have been identified in influenza viruses (N1, N2, N3, N4, N5, N6, N7, N8 and N9), all of which have been found among wild birds. NA is involved in the destruction of the cellular receptor for the viral HA by cleaving terminal neuraminic acid (also called sialic acid) residues from carbohydrate moieties on the surfaces of infected cells. NA also cleaves sialic acid residues from viral proteins, preventing aggregation of viruses. Using this mechanism, it is hypothesized that NA facilitates the release of viral progeny by preventing newly formed viral particles from accumulating along the cell membrane, as well as by promoting transportation of the virus through the mucus present on the mucosal surface. NA is an important antigenic determinant that is subject to antigenic variation.

In addition to the surface proteins HA and NA, influenza virus comprises six additional internal genes, which give rise to eight different proteins, including polymerase genes PB1, PB2 and PA, matrix proteins M1 and M2, nucleoprotein (NP), and non-structural proteins NS1 and NS2 (See, e.g., Horimoto et al., 2001, Cin Microbiol Rev. 14(1):129-149).

For packaging into progeny virions, viral RNA is transported from the nucleus as a ribonucleoprotein (RNP) complex composed of the three influenza virus polymerase proteins, the nucleoprotein (NP), and the viral RNA, in association with the influenza virus matrix 1 (M1) protein and nuclear export protein (Marsh et al., 2008, J Virol, 82:2295-2304). The M1 protein that lies within the envelope is thought to function in assembly and budding. A limited number of M2 proteins are integrated into the virions (Zebedee, 1988, J. Virol. 62:2762-2772). These M2 proteins form tetramers having H+ ion channel activity, and when activated by the low pH in endosomes, acidify the inside of the virion, thus facilitating its uncoating (Pinto et al., 1992, Cell 69:517-528). Amantadine is an anti-influenza drug that prevents viral infection by interfering with M2 ion channel activity, thus inhibiting virus uncoating.

NS1, a nonstructural protein, has multiple functions, including regulation of splicing and nuclear export of cellular mRNAs as well as stimulation of translation. The major function of NS1 seems to be to counteract the interferon activity of the host, since an NS1 knockout virus was viable, although it grew less efficiently than the parent virus in interferon-nondefective cells (Garcia-Sastre, 1998, Virology 252:324-330).

The NS2 nonstructural protein has been detected in virus particles (Richardson et al., 1991, Arch. Virol. 116:69-80; Yasuda et al., 1993, Virology 196:249-255). The average number of NS2 proteins in a virus particle was estimated to be 130-200 molecules. An in vitro binding assay has demonstrated direct protein-protein contact between M1 and NS2. NS2-M1 complexes have also been detected by immunoprecipitation in virus-infected cell lysates. The NS2 protein is thought to play a role in the export of the RNP from the nucleus through interaction with M1 protein (Ward et al., 1995, Arch. Virol. 140:2067-2073).

### Viral Proteins and Virus-Like Particles (VLPs)

Provided are non-naturally occurring, broadly reactive influenza (e.g., H1 or H3) HA immunogenic polypeptides (immunogens) and virus-like particles (VLPs) comprising an influenza virus HA immunogen containing diverse epitopes (antigenic determinants) that endow the HA antigen with the ability to generate a broadly active immune response against influenza and its symptoms, either prophylactic or therapeutic, following administration and delivery to a susceptible subject. By way of example, representative influenza virus HA immunogenic antigen sequences are presented in SEQ ID NOS: 1-17 herein (Example 1). In particular embodiments, the broadly reactive HA polypeptides are administered as part of a VLP. In embodiments, the VLP contains one or more polynucleotides the encode one or more broadly-reactive influenza HA immunogenic antigens as described herein.

It will be understood that the influenza virus immunogens and sequences described and provided herein are non-naturally occurring and broadly reactive, whether or not these characteristics and features are explicitly stated. It will be further understood that the antigen proteins described herein and used as immunogens are non-naturally occurring or synthetic antigens that elicit an immune response, e.g., neutralizing antibodies and/or a cellular immune response, in a subject.

The influenza VLPs include the viral HA, NA and M1 proteins. The production of influenza VLPs has been described in the art and is within the skill and expertise of one of ordinary skill in the art. Briefly, and as described, influenza VLPs can be produced by transfection of host cells with one or more plasmids containing polynucleotide sequences that encode the HA, NA and M1 proteins. After incubation of the transfected cells for an appropriate time to allow for protein expression (such as for approximately 72 hours), VLPs can be isolated from cell culture supernatants. Influenza VLPs can be purified from cell supernatants using procedures practiced in the art, for example, VLPs can be isolated by low speed centrifugation (to remove cell debris), vacuum filtration and ultracentrifugation through 20% glycerol. In an embodiment, VLPs containing broadly reactive antigens derived from other pathogens can also be produced, isolated and used as immunogens or in immunogenic compositions.

The influenza VLPs can be used as influenza vaccines to elicit an immune response against the H1 or H3 influenza viruses. In particular, the component, broadly reactive influenza HA polypeptides of the vaccines (or VLPs) contain antigenic determinants that are broadly reactive and serve to elicit an immune response in a subject (e.g., the production of neutralizing antibodies and/or activated T-cells) that can treat a virus-infected subject (e.g., neutralize the infecting virus) and/or protect a subject against full-blown virus infection or the signs and symptoms thereof.

In an embodiment, the antigen sequence of a broadly reactive and immunogenic influenza antigen as described herein, such as an H1 or H3 HA antigen, contains a diverse repertoire of epitopic determinants that can reflect antigenic drift and sequence variability in the virus's antigenic proteins. In particular, an influenza virus HA antigen as described herein can comprise an amino acid sequence that contains antigenic determinants (epitopes) derived from sequence diverse influenza virus strains, including drift variants, against which broadly reactive neutralizing antibodies can be raised, especially when the antigen is used as an immunogenic product, (an immunogen), e.g., an antiviral vaccine, that is introduced into a subject. In an embodiment, the H1 or H3 immunogenic antigen sequences are as set forth in SEQ ID NOS: 1-17. In an embodiment, the HA immunogenic antigen is a full length HA polypeptide. In an embodiment, the HA immunogenic antigen is a soluble HA polypeptide (sHA), which lacks a transmembrane domain and a tail domain.

Because the broadly reactive influenza HA antigens and the sequences thereof as described herein and used as an immunogen or immunogenic composition, such as a vaccine, elicit a broadly reactive immune response in an immunocompetent subject, they provide a superior immunogenic product (e.g, a vaccine) that captures the antigenic determinants of different influenza isolates (subtypes or strains), against which broadly active immune responses (e.g., broadly active neutralizing antibodies and/or cellular immune responses) are generated. It is noted that the terms “broadly active” and “broadly reactive” are used synonymously herein.

In an embodiment, the influenza virus antigen as described herein is a polypeptide or peptide antigen of the virus which currently causes disease or infection and its symptoms, such as influenza, flu, or infectious bronchitis. In another embodiment, the influenza virus antigen is a polypeptide or peptide antigen which may cause future disease and infection. In an embodiment, the influenza virus antigen is a polynucleotide sequence. In an embodiment, the influenza virus antigen is a polynucleotide sequence that encodes a polypeptide or peptide antigen as described herein. By way of example, representative broadly reactive influenza virus HA immunogenic sequences are provided in SEQ ID NOS: 1-17 in Example 1 infra.

In another embodiment, the influenza immunogen sequence described herein is expressed in a cell as a polypeptide, protein, or peptide. In an embodiment, the influenza immunogen is isolated and/or purified. In an embodiment, the immunogen is formulated for administration to a subject in need. In an embodiment, the immunogen is administered to a subject in need thereof in an effective amount to elicit an immune response in the subject. In an embodiment, the immune response elicits neutralizing antibodies. In an embodiment, a cellular immune response is elicited. In an embodiment, the immune response is prophylactic or therapeutic.

In an embodiment, a non-naturally occurring influenza virus immunogen (immunogen sequence), e.g., a vaccine, is provided that elicits a broadly reactive immune response in a subject following introduction, administration, or delivery of the immunogen to the subject. The route of introduction, administration, or delivery is not limited and may include, for example, intravenous, subcutaneous, intramuscular, oral, etc. routes. The vaccine may be therapeutic (e.g., administered to a subject following a symptom of disease (flu or bronchitis) caused by the influenza virus, or it may be prophylactic (protective), (e.g., administered to a subject prior to the subject having or expressing a symptom of disease (flu or bronchitis), or full-blown disease, caused by the virus.

In an embodiment, the final amino acid sequence of the viral antigen, e.g., HA, is reverse translated and optimized for expression in mammalian cells. As will be appreciated by the skilled practitioner in the art, optimization of the nucleic acid sequence includes optimization of the codons for expression of a sequence in mammalian cells and RNA optimization (such as RNA stability).

In an embodiment, an isolated nucleic acid molecule (polynucleotide) comprising a nucleotide sequence encoding a polypeptide or peptide antigen, such as an influenza virus HA polypeptide, is provided. In certain embodiments, the nucleotide sequence encoding the HA polypeptide is at least 94%, at least 95%, at least 96%, at least 97%, at least 98% or at least 99% identical to a polynucleotide encoding an HA polypeptide sequence of SEQ ID NOS: 1-17 herein.

In other embodiments, the nucleotide sequence encoding an influenza virus HA polypeptide that is at least 90%, at least 91%, at least 92%, at least 93%, at least 94%, at least 95%, at least 96%, at least 97%, at least 98%, or at least 99% identical to a polynucleotide encoding an influenza virus HA polypeptide sequence of SEQ ID NOS: 1-17 herein lacks the start codon encoding an N-terminal methionine. In some embodiments, the nucleotide sequence encodes the start codon encoding an N-terminal methionine.

Vectors containing a nucleotide sequence encoding a non-naturally occurring, broadly reactive polypeptide or peptide antigen, such as an influenza HA polypeptide, are provided. In some embodiments, the vectors comprise a nucleotide sequence encoding the polypeptide or peptide antigen, such as an influenza H1 or H3 HA polypeptide antigen, that is at least 94%, at least 95%, at least 96%, at least 97%, at least 98% or at least 99% identical to a polynucleotide encoding an HA polypeptide sequence of SEQ ID NOS: 1-17 herein. In some embodiments, the vector further includes a promoter operably linked to the nucleotide sequence encoding the HA polypeptide. In a particular embodiment, the promoter is a cytomegalovirus (CMV) promoter. In some embodiments, the nucleotide sequence of the vector is at least 85%, at least 90%, at least 95%, at least 98% or at least 99% identical to a polynucleotide encoding an HA polypeptide sequence of SEQ ID NOS: 1-17 herein. In particular embodiments, the nucleotide sequence of the vector comprises the polynucleotide encoding an HA polypeptide sequence of SEQ ID NOS: 1-17 herein. In embodiments, the vector is a prokaryotic or eukaryotic vector. In an embodiment, the vector is an expression vector, such as a eukaryotic (e.g., mammalian) expression vector. In another embodiment, the vector is a plasmid (prokaryotic or bacterial) vector. In another embodiment, the vector is a viral vector.

The vectors used to express an influenza virus antigen, e.g., an H1 or H3 viral protein, such as the HA proteins as described herein, may be any suitable expression vectors known and used in the art. The vectors can be, for example, mammalian expression vectors or viral vectors. In some embodiments, the vector is the pTR600 expression vector (U.S. Patent Application Publication No. 2002/0106798, herein incorporated by reference; Ross et al., 2000, Nat Immunol. 1(2):102-103; and Green et al., 2001, Vaccine 20:242-248).

Provided are non-naturally occurring polypeptide immunogens derived from influenza virus, e.g., H1 or H3 influenza HA polypeptide antigens, produced by transfecting a host cell with an expression vector as known and used in the art under conditions sufficient to allow for expression of the HA polypeptide, in the cell. Isolated cells containing the vectors are also provided.

Also provided are non-naturally occurring, broadly reactive influenza virus antigen polypeptides as described herein, such as broadly reactive H1 or H3 influenza HA polypeptides. In certain embodiments, the amino acid sequence of the polypeptide is at least 95% to 99% (inclusive) identical to the amino acid sequence of an HA polypeptide as shown in SEQ ID NOS: 1-17 herein (Example 1). In particular embodiments, the amino acid sequence of the influenza HA polypeptide that is at least 95% to 99% (inclusive) identical to the amino acid sequence of an HA polypeptide of SEQ ID NOS: 1-17 lacks the N-terminal methionine residue. In a particular embodiment, the amino acid sequence of the influenza HA polypeptide is at least 95% to 99% (inclusive) identical to the amino acid sequence of the HA polypeptides of SEQ ID NOS: 1-17.

In some embodiments, fusion proteins comprising the broadly reactive influenza virus antigen polypeptides described herein, e.g., without limitation, the HA polypeptides disclosed herein, are also provided. In some embodiments, the influenza HA polypeptide can be fused to any heterologous amino acid sequence to form the fusion protein. By way of example, HA1 and HA2 polypeptides may be generated independently and then fused together to produce an influenza HA polypeptide antigen.

Also provided are virus-like particles (VLPs), in particular, H1 or H3 influenza VLPs containing a broadly reactive protein antigen, e.g., HA protein, as described herein. In certain embodiments, the HA protein of the VLP is at least or equal to 94%, at least or equal to 95%, at least or equal to 96%, at least or equal to 97%, at least or equal to 98%, at least or equal to 99% or 100% identical to the influenza virus HA proteins as set forth in SEQ ID NOS: 1-17 herein. The virus or influenza VLPs can further include any additional viral or influenza proteins necessary to form the virus particle. In certain embodiments, the virus or influenza VLPs further include influenza neuraminidase (NA) protein, influenza matrix (M1) protein, or both.

Also provided is an influenza VLP containing an H1 or H3 influenza virus HA polypeptide as described herein, produced by transfecting a host cell with a vector containing a polynucleotide encoding the HA polypeptide. In embodiments, the polynucleotide is DNA or RNA, e.g., mRNA. Also provided in a certain embodiment is an influenza VLP containing an influenza HA polypeptide as described herein, produced by transfecting a host cell with a vector encoding the influenza virus HA polypeptide, a vector encoding an influenza NA protein and a vector encoding an influenza M1 protein, under conditions sufficient to allow for expression of the influenza virus HA, NA and M1 proteins. Such VLPs comprise the sequences as set forth in SEQ ID NOS: 1-17 and are used as immunogens generate antibodies having high hemagglutinin inhibition (HAI) titers against different strains of the influenza virus types described herein.

Collections of plasmids (vectors) are also contemplated. In certain embodiments, the collection of plasmids includes a plasmid encoding an influenza virus NA, a plasmid encoding an influenza MA, and a plasmid encoding a broadly reactive influenza virus HA protein as described herein. In some embodiments, the nucleotide sequence encoding an influenza HA protein of the HA-encoding plasmid is at least 94%, at least 95%, at least 96%, at least 97%, at least 98% or at least 99% identical to a polynucleotide encoding an HA amino acid sequence as shown in SEQ ID NOS: 1-17. In some embodiments, the nucleotide sequence encoding a codon-optimized influenza HA protein of the HA-encoding plasmid is at least 94%, at least 95%, at least 96%, at least 97%, at least 98% or at least 99% identical to a polynucleotide encoding an influenza HA amino acid sequence as shown in SEQ ID NOS: 1-17. In another embodiment, the collection of plasmids contains a plasmid encoding a broadly reactive HA protein as described herein, comprising a polynucleotide encoding an HA amino acid sequence as shown in SEQ ID NOS: 1-17.

In the context of the present disclosure, “broadly reactive” or “broadly active” refers to an influenza virus protein (e.g., an H1 or an H3 HA protein sequence) that is immunogenic and contains a diversity of epitopes (antigenic determinants) that elicit in a subject an immune response (e.g., neutralizing antibodies directed against the epitopes contained in the broadly reactive protein immunogen, frequently accompanied by a T-cell response) sufficient to treat disease or infection, and/or to inhibit, neutralize, or prevent infection, caused by most or all of the influenza viruses within a specific subtype, or by related virus strains. In embodiments, the broadly reactive, H1 or H3 influenza virus-derived HA antigen protein can elicit a protective immune response against most or all known H1 or H3 influenza virus isolates, such as about 70%, about 75%, about 80%, about 85%, about 90%, about 95%, or about 96%-99% of the known H1 or H3 influenza virus isolates. In particular embodiments, the broadly reactive H1 or H3 influenza virus-derived antigen protein, e.g., HA protein, can elicit a protective immune response against most or all known H1 or H3 influenza virus isolates, such as about 70%, about 75%, about 80%, about 85%, about 90%, about 95%, or about 96%-99% of the known H1 or H3 influenza virus isolates.

### Compositions and Pharmaceutical Compositions for Administration

Compositions comprising a broadly reactive influenza HA protein, or a fusion protein or VLP comprising such a broadly reactive influenza or HA protein as described herein are provided. In some embodiments, the compositions further comprise a pharmaceutically acceptable carrier, excipient, or vehicle. In some embodiments, an adjuvant (a pharmacological or immunological agent that modifies or boosts an immune response, e.g. to produce more antibodies that are longer-lasting) is also employed. For example, without limitation, the adjuvant can be an inorganic compound, such as alum, aluminum hydroxide, or aluminum phosphate; mineral or paraffin oil; squalene; detergents such as Quil A; plant saponins; Freund's complete or incomplete adjuvant, a biological adjuvant (e.g., cytokines such as IL-1, IL-2, or IL-12); bacterial products such as killed Bordetella pertussis, or toxoids; or immuno-stimulatory oligonucleotides (such as CpG oligonucleotides).

Compositions and preparations (e.g., physiologically or pharmaceutically acceptable compositions) containing the non-naturally occurring, broadly reactive influenza virus HA polypeptides and influenza virus-like particles (VLPs) for parenteral administration include, without limitation, sterile aqueous or non-aqueous solutions, suspensions, and emulsions. Nonlimiting examples of non-aqueous solvents include propylene glycol, polyethylene glycol, vegetable oils, such as olive oil and canola oil, and injectable organic esters, such as ethyl oleate. Aqueous carriers include water, alcoholic/aqueous solutions, emulsions or suspensions, including saline and buffered media. Parenteral vehicles include, for example, sodium chloride solution, Ringer's dextrose, dextrose and sodium chloride, lactated Ringer's, or fixed oils. Intravenous vehicles include, for example, fluid and nutrient replenishers, electrolyte replenishers (such as those based on Ringer's dextrose), and the like. Preservatives and other additives may also be present in such compositions and preparations, such as, for example, antimicrobials, antioxidants, chelating agents, colorants, stabilizers, inert gases and the like.

Some of the compositions may potentially be administered as a pharmaceutically acceptable acid- or base-addition salt, formed by reaction with inorganic acids, such as hydrochloric acid, hydrobromic acid, perchloric acid, nitric acid, thiocyanic acid, sulfuric acid, and phosphoric acid, and organic acids, such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, tri-alkyl and aryl amines and substituted ethanolamines.

Provided herein are pharmaceutical compositions which include a therapeutically effective amount of a non-naturally occurring, broadly reactive influenza virus protein HA antigen, or influenza VLPs, alone, or in combination with a pharmaceutically acceptable carrier. In embodiments, the influenza virus HA antigens include those of the H1 or H3 influenza viruses having the sequences as shown, for example, in SEQ ID NOS: 1-17 herein. Pharmaceutically acceptable carriers include, but are not limited to, saline, buffered saline, dextrose, water, glycerol, ethanol, and combinations thereof. The carrier and composition can be sterile, and the formulation suits the mode of administration. The composition can also contain minor amounts of wetting or emulsifying agents, or pH buffering agents. The composition can be a liquid or aqueous solution, suspension, emulsion, dispersion, tablet, pill, capsule, powder, or sustained release formulation. A liquid or aqueous composition can be lyophilized and reconstituted with a solution or buffer prior to use. The composition can be formulated as a suppository, with traditional binders and carriers such as triglycerides. Oral formulations can include standard carriers, such as pharmaceutical grades of mannitol, lactose, starch, magnesium stearate, sodium saccharine, cellulose, and magnesium carbonate. Any of the commonly known pharmaceutical carriers, such as sterile saline solution or sesame oil, can be used. The medium can also contain conventional pharmaceutical adjunct materials such as, for example, pharmaceutically acceptable salts to adjust the osmotic pressure, buffers, preservatives and the like. Other media that can be used in the compositions and administration methods as described are normal saline and sesame oil.

### Methods of Treatment, Administration and Delivery

Methods of treating a disease or infection, or symptoms thereof, caused by influenza virus (e.g., H1 or H3 influenza viruses) are provided. The methods comprise administering a therapeutically effective amount of a broadly reactive immunogen as described herein or a pharmaceutical composition comprising the immunogen, or a vaccine (e.g., a VLP vaccine) as described herein to a subject (e.g., a mammal), in particular, a human subject, a non-human animal or veterinary subject, or an avian subject. It will be appreciated that the term administering embraces inoculation, immunization, or vaccination of a subject. The terms immunization and vaccination (“immunization/vaccination”) may be used interchangeably herein.

One embodiment involves a method of treating a subject suffering from, or at risk of or susceptible to, disease or infection, or a symptom thereof, caused by influenza virus. The method includes administering to the subject (e.g., a mammalian subject), an amount or a therapeutic amount of an immunogenic composition or a vaccine comprising a non-naturally occurring, broadly reactive influenza virus antigen polypeptide, such as the HA polypeptides or VLPs, sufficient to treat the disease, infection, or symptoms thereof, caused by the influenza virus, under conditions in which the disease, infection, and/or the symptoms thereof are treated.

In an embodiment, the methods herein include administering to the subject (including a human subject or a non-human subject identified as being in need of such treatment) an effective amount of a non-naturally occurring, broadly reactive influenza virus antigen polypeptide, such as the H1 or H3 influenza virus HA polypeptide as described herein, or a vaccine, or a composition as described herein to produce an immune response. The treatment methods are suitably administered to subjects, particularly humans, suffering from, having, susceptible to, or at risk of having a disease, disorder, infection, or symptom thereof, e.g., flu or influenza, or infectious bronchitis. In embodiments, the treatment methods are also suitably administered to non-human subjects, such as non-human animal subjects, veterinary subjects, or avian subjects. Identifying a subject in need of such treatment can be based on the judgment of the subject or of a medical or veterinary health care professional and can be subjective (e.g. opinion) or objective (e.g. measurable by a test or diagnostic method). Briefly, the determination of those subjects who are in need of treatment or who are “at risk” or “susceptible” can be made by any objective or subjective determination by a diagnostic test (e.g., genetic test, enzyme or protein marker assay), marker analysis, family history, and the like, including an opinion of the subject or a health care provider. The non-naturally occurring, broadly reactive virus immunogens, such as the H1 or H3 influenza virus HA polypeptide, immunogens and vaccines as described herein, may also be used in the treatment of any other disorders in which infection or disease caused by an H1 or H3 influenza virus may be implicated. A subject undergoing treatment can be a non-human mammal, such as a veterinary subject, an avian subject, or a human subject (also referred to as a “patient”).

In addition, prophylactic methods of preventing or protecting against a disease or infection, or symptoms thereof, caused by influenza virus, e.g., the H1 or H3 influenza viruses, are provided. Such methods comprise administering a therapeutically effective amount of a pharmaceutical composition comprising an H1 or H3 influenza virus HA polypeptide immunogenic composition or vaccine (e.g., an H1 or H3 influenza virus VLP vaccine) as described herein to a subject (e.g., a mammal such as a human) in need, in particular, prior to infection of the subject or prior to onset of the disease, such as an H1 or an H3 virus-associated disease.

In another embodiment, a method of monitoring the progress of an influenza virus infection or disease caused by, for example, H1 or H3 influenza virus, or of monitoring treatment of the influenza virus infection or disease is provided. The method includes determining a level of a diagnostic marker or biomarker (e.g., an influenza virus protein, such as H1 or H3 HA), or a diagnostic measurement (e.g., screening assay or detection assay) in a subject suffering from or susceptible to infection, disease or symptoms thereof associated with influenza virus, in which the subject has been administered an amount (e.g., a therapeutic amount) of a non-naturally occurring, broadly reactive influenza virus HA protein immunogen as described herein, or a vaccine as described herein, sufficient to treat the infection, disease, or symptoms thereof. The level or amount of the marker or biomarker (e.g., viral protein) determined in the method can be compared to known levels of the marker or biomarker in samples from healthy (uninfected), normal controls; in a pre-infection or pre-disease sample of the subject; or in other afflicted/infected/diseased patients to establish the treated subject's disease status. For monitoring, a second level or amount of the marker or biomarker in in a sample obtained from the subject is determined at a time point later than the determination of the first level or amount, and the two marker or biomarker levels or amounts can be compared to monitor the course of disease or infection, or the efficacy of the therapy/treatment. In certain embodiments, a pre-treatment level of the marker or biomarker in the subject (e.g., in a sample obtained from the subject) is determined prior to beginning treatment as described; this pre-treatment level of marker or biomarker can then be compared to the level of the marker or biomarker in the subject after the treatment commences and/or during the course of treatment to determine the efficacy of (monitor the efficacy of) the disease treatment. In the described methods, a subject may be a human subject or patient, or a non-human animal or veterinary subject.

The non-naturally occurring, broadly reactive influenza virus polypeptides, such as H1 or H3 influenza virus HA polypeptides, as described, and VLPs comprising such HA polypeptides, or compositions thereof, can be administered to a subject by any of the routes normally used for introducing a recombinant protein, composition containing the recombinant protein, or recombinant virus into a subject. Routes and methods of administration include, without limitation, intradermal, intramuscular, intraperitoneal, intrathecal, parenteral, such as intravenous (IV) or subcutaneous (SC), vaginal, rectal, intranasal, inhalation, intraocular, intracranial, or oral. Parenteral administration, such as subcutaneous, intravenous or intramuscular administration, is generally achieved by injection (immunization). Injectables can be prepared in conventional forms and formulations, either as liquid solutions or suspensions, solid forms (e.g., lyophilized forms) suitable for solution or suspension in liquid prior to injection, or as emulsions. Injection solutions and suspensions can be prepared from sterile powders, granules, and tablets. Administration can be systemic or local.

The non-naturally occurring, broadly reactive influenza virus polypeptides, such as H1 or H3 influenza virus HA polypeptides as described, and VLPs comprising such HA polypeptides, or compositions thereof, can be administered in any suitable manner, such as with pharmaceutically acceptable carriers as described supra. Pharmaceutically acceptable carriers are determined in part by the particular immunogen or composition being administered, as well as by the particular method used to administer the composition. Accordingly, a pharmaceutical composition comprising the immunogenic non-naturally occurring influenza virus antigen polypeptides, such as H1 or H3 influenza virus HA polypeptides as described, and VLPs comprising such HA polypeptides, or compositions thereof, can be prepared using a wide variety of suitable and physiologically and pharmaceutically acceptable formulations.

Administration of the broadly reactive, immunogenic virus antigen polypeptides, such as H1 or H3 influenza virus HA polypeptides as described, and VLPs comprising such HA polypeptides, or compositions thereof, can be accomplished by single or multiple doses. The dose administered to a subject should be sufficient to induce a beneficial therapeutic response in a subject over time, such as to inhibit, block, reduce, ameliorate, protect against, or prevent disease or infection by influenza virus (e.g., H1 or H3 influenza virus). The dose required will vary from subject to subject depending on the species, age, weight and general condition of the subject, by the severity of the infection being treated, by the particular composition being used and by the mode of administration. An appropriate dose can be determined by a person skilled in the art, such as a clinician or medical practitioner, using only routine experimentation.

Further provided is a method of eliciting an immune response to influenza virus in a subject by administering to the subject a non-naturally occurring, broadly reactive H1 or H3 influenza virus HA polypeptide as described, or VLPs comprising such HA polypeptides, compositions thereof, or fusion proteins containing the H1 or H3 influenza virus HA polypeptides as described herein. In some embodiments, the influenza virus HA protein, fusion protein, or VLP can be administered using any suitable route of administration, such as, for example, by intramuscular injection. In some embodiments, the influenza virus HA protein, fusion protein, or VLP is administered as a composition comprising a pharmaceutically acceptable carrier. In some embodiments the composition comprises an adjuvant selected from, for example, alum, Freund's complete or incomplete adjuvant, a biological adjuvant or immuno-stimulatory oligonucleotides (such as CpG oligonucleotides). In other embodiments, the composition may be administered in combination with another therapeutic agent or molecule, e.g., an antiviral agent or combinations thereof, as used by the skilled practitioner in the art.

Also provided is a method of immunizing a subject against infection or disease or the symptoms thereof caused by H1 or H3 influenza virus in which the method involves administering to the subject VLPs containing a non-naturally occurring, broadly reactive H1 or H3 influenza HA protein as described herein, or administering an immunogenic composition thereof. In some embodiments of the method, the composition further comprises a pharmaceutically acceptable carrier and/or an adjuvant. For example, the adjuvant can be alum, Freund's complete or incomplete adjuvant, a biological adjuvant or immuno-stimulatory oligonucleotides (such as CpG oligonucleotides). In an embodiment, the VLPs (or compositions thereof) are administered intramuscularly.

In some embodiments of the methods of eliciting an immune response or immunizing a subject against virus infection or disease caused by or associated with influenza virus (e.g., H1 or H3 influenza), the subject is administered at least 1 μg of the VLPs containing a non-naturally occurring, broadly reactive influenza virus (e.g., H1 or H3 influenza) HA protein, such as at least 5 μg, at least 10 μg, at least 15 μg, at least 20 μg, at least 25 μg, at least 30 μg, at least 40 μg g or at least 50 μg of the VLPs containing the non-naturally occurring, broadly reactive influenza virus HA protein, for example about 1 to about 50 μg or about 1 to about 25 μg of the VLPs containing the influenza virus HA protein. In particular examples, the subject is administered about 5 to about 20 μg of the VLPs, or about 10 to about 15 μg of the VLPs. In a specific, yet nonlimiting example, the subject is administered about 15 μg of the VLPs. However, one of skill in the art is capable of determining a therapeutically effective amount of VLPs (for example, an amount that provides a therapeutic effect or protection against influenza virus (e.g., H1 or H3 influenza) infection suitable for administering to a subject in need of treatment or protection from virus infection.

It is expected that the administration of VLPs comprising a non-naturally occurring, broadly reactive influenza virus HA protein as described herein will elicit high titers of neutralizing antibodies directed against the diverse repertoire of epitopic determinants on the HA protein immunogen, as well as therapeutic or protective levels of HA-inhibiting (HAI) antibodies that are directed against a number of representative influenza isolates and will provide complete protection against lethal challenge with influenza virus (e.g., H1 or H3 influenza) and/or related influenza virus types. The VLPs containing a non-naturally occurring, broadly reactive influenza HA protein (e.g., H1 or H3 influenza HA protein) as described herein, elicit a broader immune response (e.g., elicit neutralizing antibodies directed against a broader range of influenza virus isolates compared to the immune response elicited by, for example, a polyvalent influenza virus (e.g., a polyvalent H1 or H3 influenza virus) vaccine.

### Adjuvants and Combination Therapies

The influenza virus immunogens or immunogenic compositions containing an influenza protein antigen (e.g., an H1 or H3 influenza HA antigen), or containing influenza virus (e.g., H1 or H3 influenza virus) VLPs as described herein, can be administered alone or in combination with other therapeutic agents to enhance antigenicity or immunogenicity, i.e., to increase an immune response, such as the elicitation of specific antibodies, in a subject. By way of example, the H1 or H3 influenza virus VLPs can be administered with an adjuvant, such as alum, Freund's incomplete adjuvant, Freund's complete adjuvant, ADDAVAX™ adjuvant, biological adjuvant, or immuno-stimulatory oligonucleotides (such as CpG oligonucleotides). ADDAVAX™ adjuvant (InvivoGen, San Diego, CA; ThermoFisher) is a squalene-based, oil-in-water nano-emulsion with a formulation similar to that of MF59®. Such squalene oil-in-water emulsion adjuvants elicit both cellular (Th1) and humoral (Th2) immune responses and are believed to act through recruitment and activation of antigen-presenting cells (APC) and stimulation of the production of cytokines and chemokines by macrophages and granulocytes

One or more cytokines, such as interleukin-1 (IL-2), interleukin-6 (IL-6), interleukin-12 (IL-12), the protein memory T-cell attractant “Regulated on Activation, Normal T Expressed and Secreted” (RANTES), granulocyte-macrophage-colony stimulating factor (GM-CSF), tumor necrosis factor-alpha (TNF-α), or interferon-gamma (IFN-γ); one or more growth factors, such as GM-CSF or granulocyte-colony stimulation factor (G-CSF); one or more molecules such as the TNF ligand superfamily member 4 ligand (OX40L) or the type 2 transmembrane glycoprotein receptor belonging to the TNF superfamily (4-1BBL), or combinations of these molecules, can be used as biological adjuvants, if desired or warranted (see, e.g., Salgaller et al., 1998, J Surg. Oncol. 68(2):122-38; Lotze et al., 2000, Cancer J Sci. Am. 6(Suppl 1):S61-6; Cao et al., 1998, Stem Cells 16(Suppl 1):251-60; Kuiper et al., 2000, Adv. Exp. Med. Biol. 465:381-90). These molecules can be administered systemically (or locally) to a subject. In another embodiment, one or more antiviral agents, (e.g., without limitation, the influenza drugs Rapivab (peramivir), Relenza (zanamivir), Tamiflu (oseltamivir phosphate), or Xofluza (baloxavir marboxil)), as used by the skilled practitioner in the art may be co-administered to the subject.

Several ways of inducing cellular responses, both in vitro and in vivo, are known and practiced in the art. Lipids have been identified as agents capable of assisting in priming cytotoxic lymphocytes (CTL) in vivo against various antigens. For example, palmitic acid residues can be attached to the alpha and epsilon amino groups of a lysine residue and then linked (for example, via one or more linking residues, such as glycine, glycine-glycine, serine, serine-serine, or the like) to an immunogenic peptide (U.S. Pat. No. 5,662,907). The lipidated peptide can then be injected directly in a micellar form, incorporated in a liposome, or emulsified in an adjuvant. As another example, E. coli lipoproteins, such as tripalmitoyl-S-glycerylcysteinlyseryl-serine can be used to prime tumor-specific CTL when covalently attached to an appropriate peptide (see, e.g., Deres et al., 1989, Nature 342:561). Moreover, the induction of neutralizing antibodies can also be primed with the same molecule conjugated to a peptide which displays an appropriate epitope, and two compositions can be combined to elicit both humoral and cell-mediated responses where such a combination is deemed desirable.

While treatment methods may involve the administration of VLPs containing a non-naturally occurring, broadly reactive HA immunogenic protein as described herein, one skilled in the art will appreciate that the non-naturally occurring, broadly reactive HA protein itself (in the absence of a viral particle), as a component of a pharmaceutically acceptable composition, or as a fusion protein, can be administered to a subject in need thereof to elicit an immune response in the subject.

### Kits

Also provided are kits containing a non-naturally occurring, broadly reactive influenza virus immunogen as described, or a vaccine, or a pharmaceutically acceptable composition containing the immunogen and a pharmaceutically acceptable carrier, diluent, or excipient, for administering to a subject in need, for example. The immunogen may be in the form of an influenza virus (e.g., H1 or H3 influenza virus) protein (polypeptide) or polynucleotide (a polynucleotide encoding an influenza virus protein), e.g., an H1 or H3 influenza virus HA as described herein. Kits containing one or more of the plasmids, or a collection of plasmids as described herein, are also provided. As will be appreciated by the skilled practitioner in the art, such a kit may contain one or more containers that house the immunogen, vaccine, or composition, diluents or excipients, as necessary, and instructions for use.

The practice of the aspects and embodiments described herein employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the aspects and embodiments described and/or exemplified herein, and, as such, may be considered in making and practicing such aspects and embodiments. Useful techniques for particular embodiments will be discussed in the sections that follow.

### EXAMPLES

The following examples are provided to illustrate certain particular features and/or embodiments. The examples should not be construed to limit the disclosure to the particular features or embodiments described.

### Example 1: Influenza Virus Hemagglutinin (HA) Sequences

This Example presents the amino acid sequences of full length, non-naturally occurring, broadly reactive, influenza virus Hemagglutinin (HA) immunogenic polypeptide antigens and soluble influenza virus HA (sHA) antigen amino acid sequences derived from influenza H1 and H3 types, such as H1N1 and H3N2. In an embodiment, the HA antigens provide epitopes of HA antigens derived from influenza H1 or H3 in a certain time frame (e.g., a given span of years or flu seasons) that provide a broadly reactive immune response against present and future H1 or H3 HA antigens (e.g., H1 or H3 antigens of influenza viruses in circulation in future flu seasons) when the HA antigens are administered as immunogens to a subject or host. As immunogens, the non-naturally occurring HA immunogenic polypeptide antigens generate a broadly reactive immune response (antibody and/or cellular immune responses) against influenza virus (e.g., viral antigens) in a recipient subject or host. In an embodiment, the HA antigens used as immunogens generate a therapeutic and/or a protective immune response (e.g., antibody response and/or cellular immune response) in a subject against influenza virus strains or types that may arise in a different (e.g., a subsequent or future) flu season. By way of example, the non-naturally occurring, broadly reactive influenza virus hemagglutinin (HA) or neuraminidase (NA) polypeptide antigen sequences for use as immunogens may be generated by a method such as described, for example, in published PCT Application Nos. WO 2020/014673 or WO 2020/014675, the contents of which are incorporated herein by reference in their entirety. In some instances herein, the methods for generating the broadly reactive, influenza HA or NA immunogenic polypeptides (or peptides) are referred to as ‘computationally optimized broadly reactive antigen’ (Cobra) methods, and the non-naturally occurring, broadly reactive, influenza virus HA or NA immunogenic polypeptides (or peptides), including soluble forms thereof, are referred to as “Cobra” antigens or immunogens. The amino acid sequences of the H1 or H3 virus HA polypeptides provided herein are as follows:

Y2-H1N1 HA:
(SEQ ID NO: 1)
MKAILVVLLYTFTTANADTLCIGYHANNSTDTVDTVLEKNVTVTHSVNLLEDKINGKLCKLRGV
APLHLGKCNIAGWILGNPECESLSTASSWSYIVETSNSDNGTCYPGDFINYEELREQLSSVSSF
ERFEIFPKTSSWPNHDSNKGVTAACPHAGAKSFYKNLIWLVKKGNSYPKLSQSYINDKGKEVLV
LWGIHHPSTTADQQSLYQNADAYVFVGTSRYSKKFKPEIAIRPKVRDQEGRMNYYWTLVEPGDK
ITFEATGNLVVPRYAFTMERNAGSGIIISDTPVHDCNTTCQTPEGAINTSLPFQNVHPITIGKC
PKYVKSTKLRLATGLRNVPSIQSRGLFGAIAGFIEGGWTGMVDGWYGYHHQNEQGSGYAADLKS
TQNAIDKITNKVNSVIEKMNTQFTAVGKEFNHLEKRIENLNKKVDDGFLDIWTYNAELLVLLEN
ERTLDYHDSNVKNLYEKVRNQLKNNAKEIGNGCFEFYHKCDNTCMESVKNGTYDYPKYSEEAKL
NREKIDGVGTGYIPEAPRDGQAYVRKDGEWVLLSTFLGS**
NG2-H3N2 HA:
(SEQ ID NO: 2)
MKTIIALSYILCLVFAQKIPGNDNSTATLCLGHHAVPNGTIVKTITNDRIEVTNATELVQNSSI
GEICDSPHQILDGENCTLIDALLGDPQCDGFQNKKWDLFVERSKAYSNCYPYDVPDYASLRSLV
ASSGTLEFKNESFNWTGVTQNGTSSACIRGSSSSFFSRLNWLTHLNYTYPALNVTMPNNEQFDK
LYIWGVHHPGTDKDQIFLYAQSSGRITVSTKRSQQAVIPNIGSRPRIRDIPSRISIYWTIVKPG
DILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLK
STQAAIDQINGKLNRLIGKTNEKFHQIEKEFSEVEGRIQDLEKYVEDTKIDLWSYNAELLVALE
NQHTIDLTDSEMNKLFEKTKKQLRENAEDMGNGCFKIYHKCDNACIGSIRNGTYDHNVYRDEAL
NNRFQIKGVEGYIPEAPRDGQAYVRKDGEWVLLSTFLGS**
J1 (2013S-2015S) 1-566 AA:
(SEQ ID NO: 3)
MKTIIALSYILCLVFAQKIPGNDNSTATLCLGHHAVPNGTIVKTITNDRIEVTNATELVQNSSI
GEICDSPHQILDGENCTLIDALLGDPQCDGFQNKKWDLFVERSKAYSNCYPYDVPDYASLRSLV
ASSGTLEFNNESFNWTGVTQNGTSSACIRRSNSSFFSRLNWLTHLNFKYPALNVTMPNNEQFDK
LYIWGVHHPGTDKDQIFLYAQSSGRITVSTKRSQQAVIPNIGSRPRIRNIPSRISIYWTIVKPG
DILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLK
STQAAIDQINGKLNRLIGKTNEKFHQIEKEFSEVEGRIQDLEKYVEDTKIDLWSYNAELLVALE
NQHTIDLTDSEMNKLFEKTKKQLRENAEDMGNGCFKIYHKCDNACIGSIRNGTYDHNVYRDEAL
NNRFQIKGVELKSGYKDWILWISFAISCFLLCVALLGFIMWACQKGNIRCNICI
J2 (2014-15N thru 2015-16N) 1-566 AA:
(SEQ ID NO: 4)
MKTIIALSYILCLVFAQKIPGNDNSTATLCLGHHAVPNGTIVKTITNDRIEVTNATELVQNSSI
GEICDSPHQILDGENCTLIDALLGDPQCDGFQNKKWDLFVERSKAYSNCYPYDVPDYASLRSLV
ASSGTLEFNNESFNWTGVTQNGTSSACIRRSSSSFFSRLNWLTHLNYKYPALNVTMPNNEQFDK
LYIWGVHHPGTDKDQIFLYAQSSGRITVSTKRSQQAVIPNIGSRPRIRDIPSRISIYWTIVKPG
DILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLK
STQAAIDQINGKLNRLIGKTNEKFHQIEKEFSEVEGRIQDLEKYVEDTKIDLWSYNAELLVALE
NQHTIDLTDSEMNKLFEKTKKQLRENAEDMGNGCFKIYHKCDNACIGSIRNGTYDHNVYRDEAL
NNRFQIKGVELKSGYKDWILWISFAISCFLLCVALLGFIMWACQKGNIRCNICI
J3 (2014S-2016S) (S1) 1-566 AA:
(SEQ ID NO: 5)
MKTIIALSYILCLVFAQKIPGNDNSTATLCLGHHAVPNGTIVKTITNDRIEVTNATELVQNSSI
GEICDSPHQILDGENCTLIDALLGDPQCDGFQNKKWDLFVERSKAYSNCYPYDVPDYASLRSLV
ASSGTLEFNNESFNWTGVTQNGTSSACIRRSSSSFFSRLNWLTHLNYTYPALNVTMPNNEQFDK
LYIWGVHHPGTDKDQIFLYAQSSGRITVSTKRSQQAVIPNIGSRPRIRDIPSRISIYWTIVKPG
DILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLK
STQAAIDQINGKLNRLIGKTNEKFHQIEKEFSEVEGRIQDLEKYVEDTKIDLWSYNAELLVALE
NQHTIDLTDSEMNKLFEKTKKQLRENAEDMGNGCFKIYHKCDNACIGSIRNGTYDHNVYRDEAL
NNRFQIKGVELKSGYKDWILWISFAISCFLLCVALLGFIMWACQKGNIRCNICI
J4 1-566 AA (2013S thru 2015-16N) (S2):
(SEQ ID NO: 6)
MKTIIALSYILCLVFAQKIPGNDNSTATLCLGHHAVPNGTIVKTITNDRIEVTNATELVQNSSI
GEICDSPHQILDGENCTLIDALLGDPQCDGFQNKKWDLFVERSKAYSNCYPYDVPDYASLRSLV
ASSGTLEFNNESFNWTGVTQNGTSSACIRRSNSSFFSRLNWLTHLNYKYPALNVTMPNNEQFDK
LYIWGVHHPGTDKDQIFLYAQSSGRITVSTKRSQQAVIPNIGSRPRIRDIPSRISIYWTIVKPG
DILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLK
STQAAIDQINGKLNRLIGKTNEKFHQIEKEFSEVEGRIQDLEKYVEDTKIDLWSYNAELLVALE
NQHTIDLTDSEMNKLFEKTKKQLRENAEDMGNGCFKIYHKCDNACIGSIRNGTYDHNVYRDEAL
NNRFQIKGVELKSGYKDWILWISFAISCFLLCVALLGFIMWACQKGNIRCNICI
J1 HA:
(SEQ ID NO: 7)
MKTIIALSYILCLVFAQKIPGNDNSTATLCLGHHAVPNGTIVKTITNDRIEVTNATELVQNSSI
GEICDSPHQILDGENCTLIDALLGDPQCDGFQNKKWDLFVERSKAYSNCYPYDVPDYASLRSLV
ASSGTLEFNNESFNWTGVTQNGTSSACIRRSNSSFFSRLNWLTHLNFKYPALNVTMPNNEQFDK
LYIWGVHHPGTDKDQIFLYAQSSGRITVSTKRSQQAVIPNIGSRPRIRNIPSRISIYWTIVKPG
DILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLK
STQAAIDQINGKLNRLIGKTNEKFHQIEKEFSEVEGRIQDLEKYVEDTKIDLWSYNAELLVALE
NQHTIDLTDSEMNKLFEKTKKQLRENAEDMGNGCFKIYHKCDNACIGSIRNGTYDHNVYRDEAL
NNRFQIKGVELKSGYKDWILWISFAISCFLLCVALLGFIMWACQKGNIRCNICI
J4 HA:
(SEQ ID NO: 8)
MKTIIALSYILCLVFAQKIPGNDNSTATLCLGHHAVPNGTIVKTITNDRIEVTNATELVQNSSI
GEICDSPHQILDGENCTLIDALLGDPQCDGFQNKKWDLFVERSKAYSNCYPYDVPDYASLRSLV
ASSGTLEFNNESFNWTGVTQNGTSSACIRRSNSSFFSRLNWLTHLNYKYPALNVTMPNNEQFDK
LYIWGVHHPGTDKDQIFLYAQSSGRITVSTKRSQQAVIPNIGSRPRIRDIPSRISIYWTIVKPG
DILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLK
STQAAIDQINGKLNRLIGKTNEKFHQIEKEFSEVEGRIQDLEKYVEDTKIDLWSYNAELLVALE
NQHTIDLTDSEMNKLFEKTKKQLRENAEDMGNGCFKIYHKCDNACIGSIRNGTYDHNVYRDEAL
NNRFQIKGVELKSGYKDWILWISFAISCFLLCVALLGFIMWACQKGNIRCNICI
Soluble HA sequences
J1 H3N2 sHA:
(SEQ ID NO: 9)
MKTIIALSYILCLVFAQKIPGNDNSTATLCLGHHAVPNGTIVKTITNDRIEVTNATELVQNSSI
GEICDSPHQILDGENCTLIDALLGDPQCDGFQNKKWDLFVERSKAYSNCYPYDVPDYASLRSLV
ASSGTLEFNNESFNWTGVTQNGTSSACIRRSNSSFFSRLNWLTHLNFKYPALNVTMPNNEQFDK
LYIWGVHHPGTDKDQIFLYAQSSGRITVSTKRSQQAVIPNIGSRPRIRNIPSRISIYWTIVKPG
DILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLK
STQAAIDQINGKLNRLIGKTNEKFHQIEKEFSEVEGRIQDLEKYVEDTKIDLWSYNAELLVALE
NQHTIDLTDSEMNKLFEKTKKQLRENAEDMGNGCFKIYHKCDNACIGSIRNGTYDHNVYRDEAL
NNRFQIKGVEGYIPEAPRDGQAYVRKDGEWVLLSTFLGSGLNDIFEAQKIEWHEGHHHHHH**
J4 sHA:
(SEQ ID NO: 10)
MKTIIALSYILCLVFAQKIPGNDNSTATLCLGHHAVPNGTIVKTITNDRIEVTNATELVQNSSI
GEICDSPHQILDGENCTLIDALLGDPQCDGFQNKKWDLFVERSKAYSNCYPYDVPDYASLRSLV
ASSGTLEFNNESFNWTGVTQNGTSSACIRRSNSSFFSRLNWLTHLNYKYPALNVTMPNNEQFDK
LYIWGVHHPGTDKDQIFLYAQSSGRITVSTKRSQQAVIPNIGSRPRIRDIPSRISIYWTIVKPG
DILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLK
STQAAIDQINGKLNRLIGKTNEKFHQIEKEFSEVEGRIQDLEKYVEDTKIDLWSYNAELLVALE
NQHTIDLTDSEMNKLFEKTKKQLRENAEDMGNGCFKIYHKCDNACIGSIRNGTYDHNVYRDEAL
NNRFQIKGVEGYIPEAPRDGQAYVRKDGEWVLLSTFLGSGLNDIFEAQKIEWHEGHHHHHH**
NG1 sHA:
(SEQ ID NO: 11)
MKTIIALSYILCLVFAQKIPGNDNSTATLCLGHHAVPNGTIVKTITNDRIEVTNATELVQNSSI
GEICDSPHQILDGENCTLIDALLGDPQCDGFQNKKWDLFVERSKAYSNCYPYDVPDYASLRSLV
ASSGTLEFNNESFNWTGVTQNGTSSACIRGSSSSFFSRLNWLTHLNYTYPALNVTMPNNEQFDK
LYIWGVHHPGTDKDQIFLYAQSSGRITVSTKRSQQAVIPNIGSRPRIRDIPSRISIYWTIVKPG
DILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLK
STQAAIDQINGKLNRLIGKTNEKFHQIEKEFSEVEGRIQDLEKYVEDTKIDLWSYNAELLVALE
NQHTIDLTDSEMNKLFEKTKKQLRENAEDMGNGCFKIYHKCDNACIGSIRNGTYDHNVYRDEAL
NNRFQIKGVEGYIPEAPRDGQAYVRKDGEWVLLSTFLGSGLNDIFEAQKIEWHEGHHHHHH**
NG2 sHA:
(SEQ ID NO: 12)
MKTIIALSYILCLVFAQKIPGNDNSTATLCLGHHAVPNGTIVKTITNDRIEVTNATELVQNSSI
GEICDSPHQILDGENCTLIDALLGDPQCDGFQNKKWDLFVERSKAYSNCYPYDVPDYASLRSLV
ASSGTLEFKNESFNWTGVTQNGTSSACIRGSSSSFFSRLNWLTHLNYTYPALNVTMPNNEQFDK
LYIWGVHHPGTDKDQIFLYAQSSGRITVSTKRSQQAVIPNIGSRPRIRDIPSRISIYWTIVKPG
DILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLK
STQAAIDQINGKLNRLIGKTNEKFHQIEKEFSEVEGRIQDLEKYVEDTKIDLWSYNAELLVALE
NQHTIDLTDSEMNKLFEKTKKQLRENAEDMGNGCFKIYHKCDNACIGSIRNGTYDHNVYRDEAL
NNRFQIKGVEGYIPEAPRDGQAYVRKDGEWVLLSTFLGSGLNDIFEAQKIEWHEGHHHHHH**
NG3 sHA:
(SEQ ID NO: 13)
MKTIIALSYILCLVFAQKIPGNDNSTATLCLGHHAVPNGTIVKTITNDRIEVTNATELVQNSSI
GEICDSPHQILDGGNCTLIDALLGDPQCDGFQNKKWDLFVERSRAYSNCYPYDVPDYASLRSLV
ASSGTLEFKNESFNWTGVTQNGTSSACIRGSSSSFFSRLNWLTHLNYTYPALNVTMPNKEQFDK
LYIWGVHHPGTDKDQIFLYAQSSGRITVSTKRSQQAVIPNIGSRPRIRDIPSRISIYWTIVKPG
DILLINSTGNLIAPRGYFKIRSGKSSIMRSDAPIGKCKSECITPNGSIPNDKPFQNVNRITYGA
CPRYVKQSTLKLATGMRNVPEKQTRGIFGAIAGFIENGWEGMVDGWYGFRHQNSEGRGQAADLK
STQAAIDQINGKLNRLIGKTNEKFHQIEKEFSEVEGRIQDLEKYVEDTKIDLWSYNAELLVALE
NQHTIDLTDSEMNKLFEKTKKQLRENAEDMGNGCFKIYHKCDNACIGSIRNGTYDHNVYRDEAL
NNRFQIKGVEGYIPEAPRDGQAYVRKDGEWVLLSTFLGSGLNDIFEAQKIEWHEGHHHHHH**
H1N1 soluble HA Sequences
Y1 H1N1 SHA:
(SEQ ID NO: 14)
MKAILVVLLYTFTTANADTLCIGYHANNSTDTVDTVLEKNVTVTHSVNLLEDKINGKLCKLRGV
APLHLGKCNIAGWILGNPECESLSTASSWSYIVETSSSDNGTCYPGDFINYEELREQLSSVSSF
ERFEIFPKTSSWPNHDSNKGVTAACPHAGAKSFYKNLIWLVKKGNSYPKLSQSYINDKGKEVLV
LWGIHHPSTTADQQSLYQNADAYVFVGTSRYSKKFKPEIAIRPKVRDQEGRMNYYWTLVEPGDK
ITFEATGNLVVPRYAFTMERNAGSGIIISDTPVHDCNTTCQTPEGAINTSLPFQNVHPITIGKC
PKYVKSTKLRLATGLRNVPSIQSRGLFGAIAGFIEGGWTGMVDGWYGYHHQNEQGSGYAADLKS
TQNAIDKITNKVNSVIEKMNTQFTAVGKEFNHLEKRIENLNKKVDDGFLDIWTYNAELLVLLEN
ERTLDYHDSNVKNLYEKVRNQLKNNAKEIGNGCFEFYHKCDNTCMESVKNGTYDYPKYSEEAKL
NREKIDGVKLESTRIYGSGYIPEAPRDGQAYVRKDGEWVLLSTFLGLNDIFEAQKIEWHEGHHH
HHH
Y2 H1N1 sHA:
(SEQ ID NO: 15)
MKAILVVLLYTFTTANADTLCIGYHANNSTDTVDTVLEKNVTVTHSVNLLEDKINGKLCKLRGV
APLHLGKCNIAGWILGNPECESLSTASSWSYIVETSNSDNGTCYPGDFINYEELREQLSSVSSF
ERFEIFPKTSSWPNHDSNKGVTAACPHAGAKSFYKNLIWLVKKGNSYPKLSQSYINDKGKEVLV
LWGIHHPSTTADQQSLYQNADAYVFVGTSRYSKKFKPEIAIRPKVRDQEGRMNYYWTLVEPGDK
ITFEATGNLVVPRYAFTMERNAGSGIIISDTPVHDCNTTCQTPEGAINTSLPFQNVHPITIGKC
PKYVKSTKLRLATGLRNVPSIQSRGLFGAIAGFIEGGWTGMVDGWYGYHHQNEQGSGYAADLKS
TQNAIDKITNKVNSVIEKMNTQFTAVGKEFNHLEKRIENLNKKVDDGFLDIWTYNAELLVLLEN
ERTLDYHDSNVKNLYEKVRNQLKNNAKEIGNGCFEFYHKCDNTCMESVKNGTYDYPKYSEEAKL
NREKIDGVKLESTRIYGSGYIPEAPRDGQAYVRKDGEWVLLSTFLGLNDIFEAQKIEWHEGHHH
HHH
Y3 H1N1 sHA:
(SEQ ID NO: 16)
MKAILVVLLYTFTTANADTLCIGYHANNSTDTVDTVLEKNVTVTHSVNLLEDKHNGKLCKLRGV
APLHLGKCNIAGWILGNPECESLSTASSWSYIVETSNSDNGTCYPGDFINYEELREQLSSVSSF
ERFEIFPKTSSWPNHDSNKGVTAACPHAGAKSFYKNLIWLVKKGNSYPKLNQSYINDKGKEVLV
LWGIHHPSTTADQQSLYQNADAYVFVGTSRYSKKFKPEIATRPKVRDQEGRMNYYWTLVEPGDK
ITFEATGNLVVPRYAFTMERNAGSGIIISDTPVHDCNTTCQTPEGAINTSLPFQNVHPITIGKC
PKYVKSTKLRLATGLRNVPSIQSRGLFGAIAGFIEGGWTGMVDGWYGYHHQNEQGSGYAADLKS
TQNAIDKITNKVNSVIEKMNTQFTAVGKEFNHLEKRIENLNKKVDDGFLDIWTYNAELLVLLEN
ERTLDYHDSNVKNLYEKVRNQLKNNAKEIGNGCFEFYHKCDNTCMESVKNGTYDYPKYSEEAKL
NREKIDGVKLESTRIYGSGYIPEAPRDGQAYVRKDGEWVLLSTFLGLNDIFEAQKIEWHEGHHH
HHH
Y4 H1N1 sHA:
(SEQ ID NO: 17)
MKAILVVLLYTFTTANADTLCIGYHANNSTDTVDTVLEKNVTVTHSVNLLEDKINGKLCKLRGV
APLHLGKCNIAGWILGNPECESLSTARSWSYIVETSNSDNGTCYPGDFINYEELREQLSSVSSF
ERFEIFPKTSSWPNHDSNKGVTAACPHAGAKSFYKNLIWLVKKGNSYPKLNQTYINDKGKEVLV
LWGIHHPSTTADQQSLYQNADAYVFVGTSRYSKKFKPEIATRPKVRDQEGRMNYYWTLVEPGDK
ITFEATGNLVVPRYAFTMERNAGSGIIISDTPVHDCNTTCQTPEGAINTSLPFQNVHPITIGKC
PKYVKSTKLRLATGLRNVPSIQSRGLFGAIAGFIEGGWTGMVDGWYGYHHQNEQGSGYAADLKS
TQNAIDKITNKVNSVIEKMNTQFTAVGKEFNHLEKRIENLNKKVDDGFLDIWTYNAELLVLLEN
ERTLDYHDSNVKNLYEKVRNQLKNNAKEIGNGCFEFYHKCDNTCMESVKNGTYDYPKYSEEAKL
NREKIDGVKLESTRIYGSGYIPEAPRDGQAYVRKDGEWVLLSTFLGLNDIFEAQKIEWHEGHHH
HHH

### Example 2: Ferret and Mouse Animal Studies Using Recombinant Influenza Virus HA Immunogenic Peptides (Influenza a(H3)) as Immunogens (Vaccines)

Studies were performed in vivo in pre-immune and naïve mice and in pre-immune and naïve ferrets using recombinant influenza virus HA immunogenic peptides (such as Influenza A(H3N2)) as immunogens (vaccines) to determine the immunogenicity and efficacy of the recombinant influenza HA immunogens in eliciting broadly protective immune responses against seasonal and pandemic influenza viruses using an in vivo animal model. The pre-immune mouse and ferret animals provide relevant animal models in which to evaluate the efficacy of the influenza HA immunogens described herein as therapeutics in preventing and/or treating disease, as the animals normally do not exhibit pre-existing antibodies to seasonal influenza viral antigens (e.g., HA). In contrast, most human subjects have pre-existing antibodies to seasonal influenza viral antigens. As described in Example 1, the HA polypeptide immunogen(s) (and recombinant HA polypeptide immunogen(s)) constitute influenza virus Hemagglutinin (HA) antigen amino acid sequences derived from influenza H1 and H3 types, such as H1N1 and H3N2, e.g., containing HA sequences representing those of seasonal or pandemic influenza viruses. In the below examples, the terms immunization and vaccination are used interchangeably.

### Ferret Studies

### Viral Infection and Immunization (Vaccination) of Ferrets with Broadly-Reactive, Recombinant HA Polypeptide Immunogens

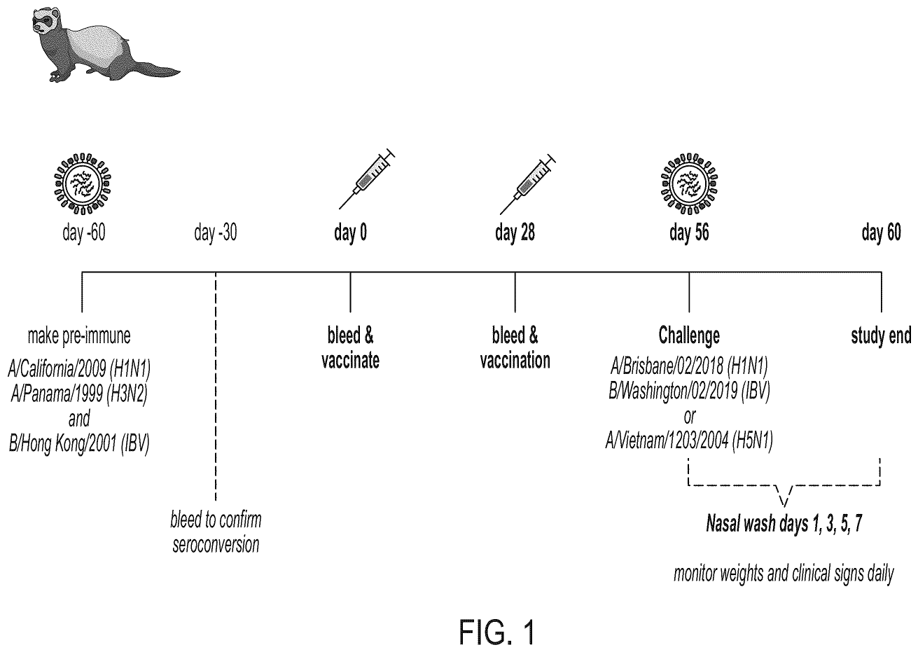

Fitch ferrets (Mustela putorius furo, female, 6 to 12 months of age), negative for antibodies to circulating influenza A (H1N1, H3N2) and influenza B viruses, were de-scented and purchased from Triple F Farms (Sayre, PA). Ferrets were pair-housed in stainless steel cages (Shor-line, Kansas City, KS) containing Sani-Chips laboratory animal bedding (P. J. Murphy Forest Products, Montville, NJ). Ferrets were provided with Teklad Global Ferret Diet (Harlan Teklad, Madison, WI) and with fresh water ad libitum. For the pre-immune vaccine groups, ferrets were infected with the CA/09, Pan/99, and B/HK/01 virus strains (10⁶ PFU each) 60 days prior to vaccination. (FIG. 1) Ferrets were vaccinated intranasally (days 0 and 28) with an octavalent formulation of recombinant influenza hemagglutinin (HA) and neuraminidase (NA) immunogenic polypeptide antigens (Y4, Z1, NG3, IAN8, Q6, BC2, N1I (also termed NA-A), and N2A), for example, the non-naturally occurring, broadly reactive influenza immunogenic polypeptides as described herein (e.g., Y4 (SEQ ID NO: 17); NG3 (SEQ ID NO: 13), as well as in WO 2020/014673 A1 (e.g., Z1; IAN8); in WO 2021/142256 A2 (e.g., Q6 (H7 HA) and neuraminidase antigens such as N1, N2, N1I (NA-A and NA-D)); in WO 2020/014656 A1, in Y. Huang et al., 2021, Vaccines, 9:793. doi.org/10.3390/vaccines9070793, or in J. D. Allen and T. M. Ross, 2022, J. Virology, Vol. 96, No. 7: doi.org/10.1128/jvi.01652-21, the contents of all of which are incorporated herein by reference in their entireties). The vaccine immunogens contained 15 μg of each antigen formulated with 50 μg cyclic-di-AMP as adjuvant (InvivoGen, San Diego, CA). Ferrets were boosted 28 days after initial vaccination. Blood was harvested from all anesthetized ferrets via the anterior vena cava prior to vaccination, and at days 28 and 56 post-initial vaccination. Sera collected from the animals was transferred to a centrifuge tube and centrifuged at 2500 rpm. Clarified serum was removed and frozen at −20±5° C. Control (mock) ferrets were immunized with placebo which included phosphate-buffered saline (PBS), pH 7.4, formulated with 50 μg of c-di-AMP. All vaccine immunogens and placebo were stored in a refrigerator at a temperature between 2° C. and 8° C. until use.

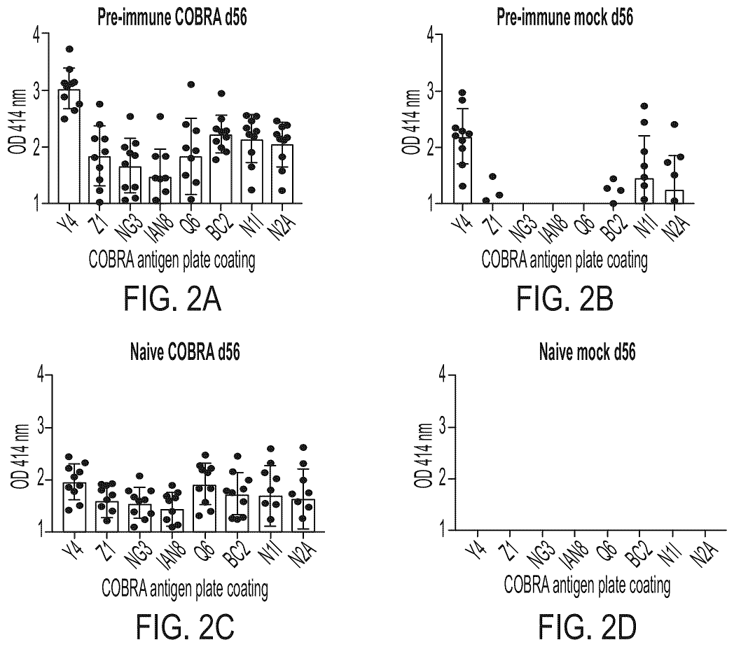

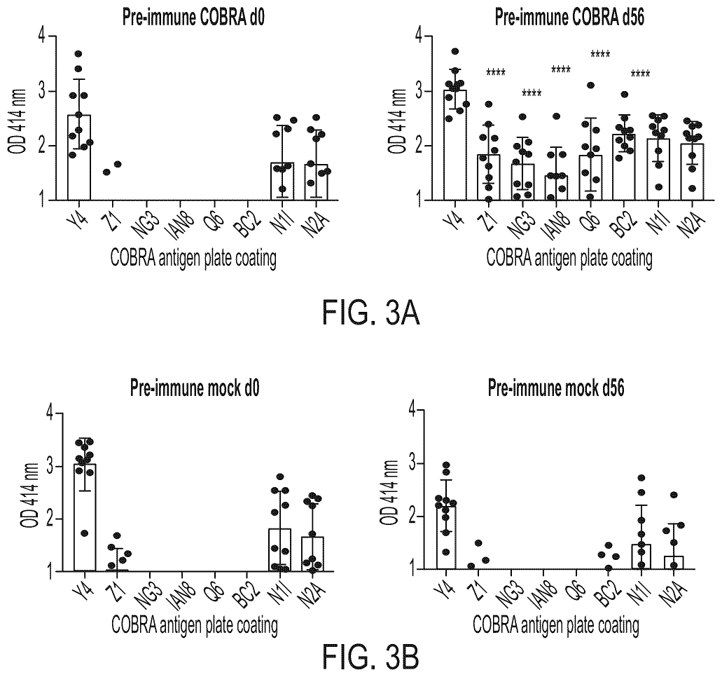

On day 56 post-vaccination, ferrets were challenged intranasally with 10⁸ PFU of Bris/18 (H1N1), 10⁷ PFU of B/WA/19 (IBV) or 10⁵ PFU of Vn/04 (H5N1) viruses in a volume of 1 ml (n=5 per vaccine group per challenge). For 14 days after infection, ferrets were monitored daily for weight loss, disease signs, and death. Individual body weights and deaths were recorded for each group on each day post virus challenge. Experimental endpoints were defined as >20% weight loss. Nasal washes were performed by instilling 3 ml of PBS into the nares of anesthetized ferrets on days 1, 3, 5, and 7 days post infection. (FIG. 1). Washes were collected and stored at −80° C. until use. The University of Georgia Institutional Animal Care and Use Committee approved all experiments under the Animal Use Protocol (no. A2020 11-016), which were conducted in accordance with the National Research Council's Guide for the Care and Use of Laboratory Animals, The Animal Welfare Act, and the CDC/NIH's Biosafety in Microbiological and Biomedical Laboratories guide. FIGS. 2A-2D present graphs showing the results of ELISA analyses performed on serum obtained from the ferrets day 56 of the study. The results demonstrated that immunization with the octavalent HA immunogenic polypeptide antigens (Cobra antigens) as described herein elicited an immune response and the production of antibodies that bound to all components of the HA immunogenic polypeptides/vaccine (FIGS. 2A and 2C). Antibodies from the pre-immune mock animals bound to the H1, N1 and N2 HA antigens (FIG. 2B). Antibodies from the naive mock animals showed no binding to the HA antigens (FIG. 2D). FIGS. 3A and 3B present graphs of the results of ELISA analyses performed to compare the total IgG antibody response before and after vaccination in pre-immune groups of ferrets on day 0 and day 56 of the study using sera obtained from pre-immune ferrets immunized with HA immunogenic polypeptide antigens or from pre-immune, mock-immunized ferrets. Sera were collected before immunization/vaccination (d0) and after final immunization/vaccination (d56) for individual pre-immune ferrets that were immunized with HA polypeptide immunogen (Cobra) (FIG. 3A) and pre-immune ferrets given mock vaccination (FIG. 3B). The results demonstrated that pre-immune ferrets that were immunized with HA immunogenic polypeptide antigens (Cobra antigens) as described herein had an increase in antibodies to all HA components of the immunogenic polypeptide antigens/vaccine, with statistically significant increases in antibodies generated against the Z1, NG3, IAN8, Q6, and BC2 immunogens.

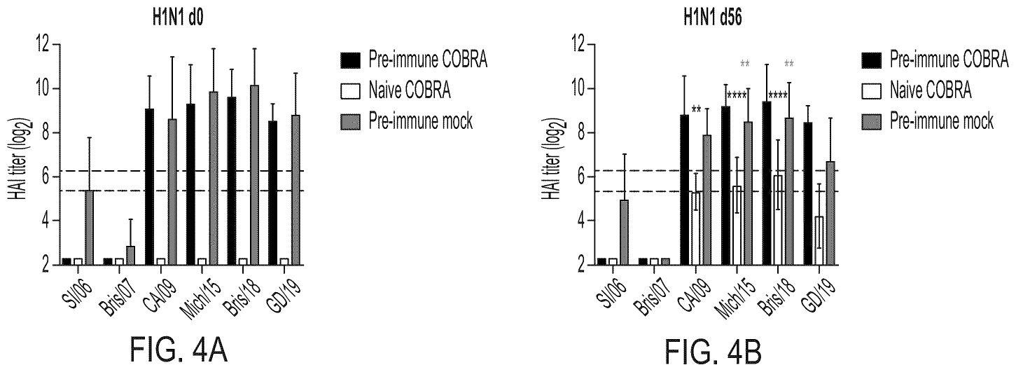

FIGS. 4A and 4B show serum HAI antibody titers for H1N1 viruses in serum obtained from pre-immune ferrets immunized with HA immunogenic polypeptide antigens (Cobra antigens), from naïve ferrets immunized with HA immunogenic polypeptide antigens (Cobra antigens), and from pre-immune, mock-immunized ferrets before and after vaccination. Ferrets were vaccinated intranasally twice at four-week intervals with c-di-AMP as adjuvant. Sera were collected before vaccination (H1N1, day 0; FIG. 4A) and 4 weeks after second vaccination (H1N1, day 56; FIG. 4B) for performing HAI assays against a panel of 6 H1N1 influenza viruses. The results showed that the pre-immune mock groups had statistically significant lower HAI titers for Mich/15 and Bris/18 strains of virus; the pre-immune groups immunized with HA immunogenic polypeptide antigens (Cobra antigens) maintained high HAI titers; and the naïve groups with HA immunogenic polypeptide antigens (Cobra antigens) reached HAI titers of 1:40 for pandemic-like strains, except Guangdong/19.

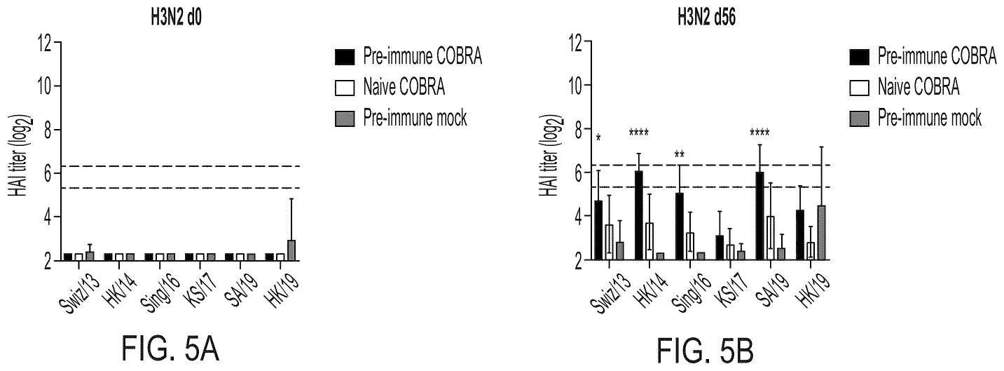

FIGS. 5A and 5B show serum HAI antibody titers for H3N2 viruses in sera obtained from study ferrets before and after vaccination. Ferrets were vaccinated intranasally twice at four-week intervals with c-di-AMP as adjuvant. Vaccine groups included pre-immune ferrets given octavalent recombinant influenza immunogenic polypeptides (Cobra antigens) (black bar); naïve ferrets given octavalent recombinant influenza immunogenic polypeptides (Cobra antigens) (white bar); or pre-immune ferrets given mock vaccination (grey bar). Sera were collected before vaccination (FIG. 5A) and 4 weeks after the second vaccination (FIG. 5B) for HAI assay against a panel of 6 H3N2 influenza viruses. The results showed that pre-immune ferrets that had received the octavalent recombinant influenza immunogenic polypeptides as immunogens had a statistically significant increase in HAI titers, except for KS/17 and HK/19, and that naïve ferrets that had received the octavalent recombinant influenza immunogenic polypeptides as immunogens had titers that increased but were not statistically significant.

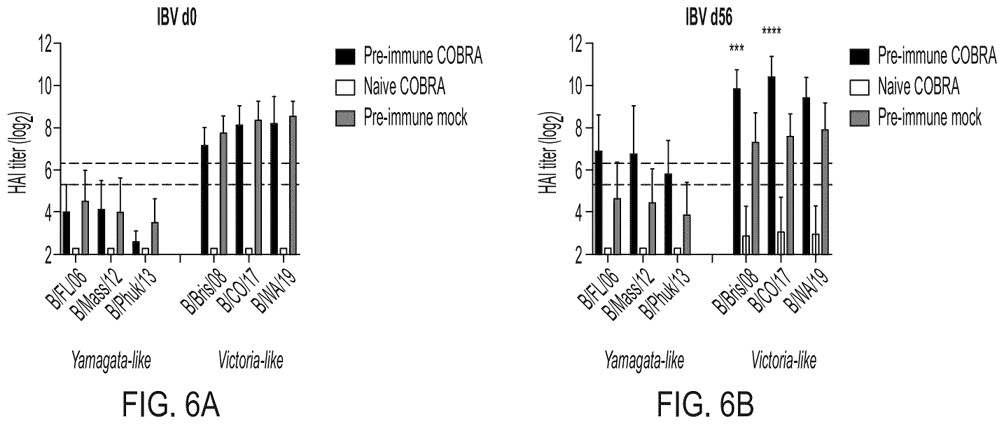

FIGS. 6A and 6B demonstrate serum HAI antibody titers in study ferrets against Influenza B (IBV) viruses before and after vaccination. Vaccine groups were: pre-immune ferrets given octavalent recombinant influenza immunogenic polypeptides (Cobra antigens); naïve ferrets given octavalent recombinant influenza immunogenic polypeptides (Cobra antigens); or pre-immune ferrets given mock vaccination. Sera were collected before vaccination and at 4 weeks after the second vaccination for HAI assay against a panel of 6 IBV influenza viruses. The results showed that the ferrets in the pre-immune groups given octavalent recombinant influenza immunogenic polypeptides (Cobra antigens) had increased HAI titers, statistically significant for B/Bris/08 and B/CO/17, and that the naïve ferrets given octavalent recombinant influenza immunogenic polypeptides (Cobra antigens) and the pre-immune mock-immunized ferrets did not have statistically significant changes.

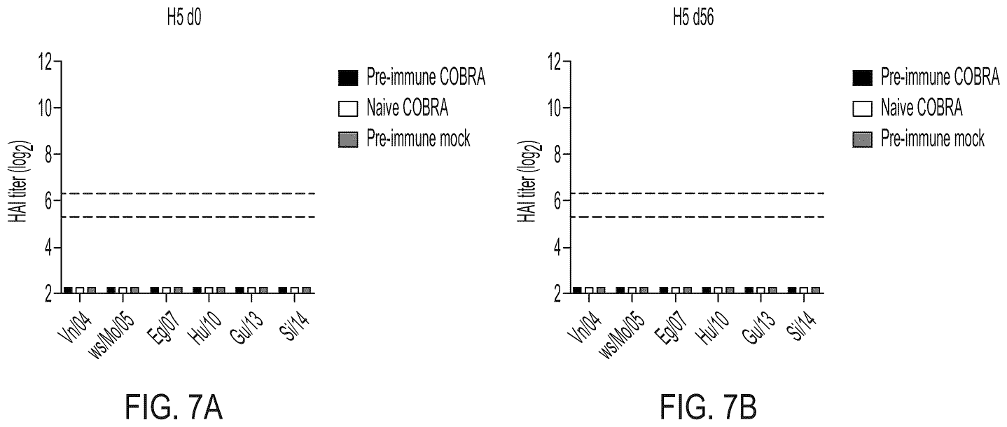

FIGS. 7A and 7B demonstrate serum HAI antibody titers in study ferrets against H5 viruses before and after immunization/vaccination. Ferrets were vaccinated intranasally twice at four-week intervals with c-di-AMP as adjuvant. Vaccine groups were: pre-immune ferrets immunized with octavalent recombinant influenza immunogenic polypeptides (Cobra antigens); naïve ferrets immunized with octavalent recombinant influenza immunogenic polypeptides (Cobra antigens); or pre-immune ferrets given mock vaccination. Sera were collected before immunization/vaccination and at 4 weeks after the second immunization/vaccination for HAI assay against a panel of 6 H5 influenza viruses. A/Vietnam/1203/2004 (H5N1, Vn/04), A/whooper swan/Mongolia/244/2005 (H5N1, ws/Mo/05) A/Egypt/321/2007 (H5N1, Eg/07), A/Hubei/01/2010 (H5N1, Hu/10), A/Guizhou/01/2013 (H5N1, Gu/13). A/Sichuan/26221/2014 (H5N6, Si/14). The results showed that none of the groups had HAI titers to H5N1 viruses.

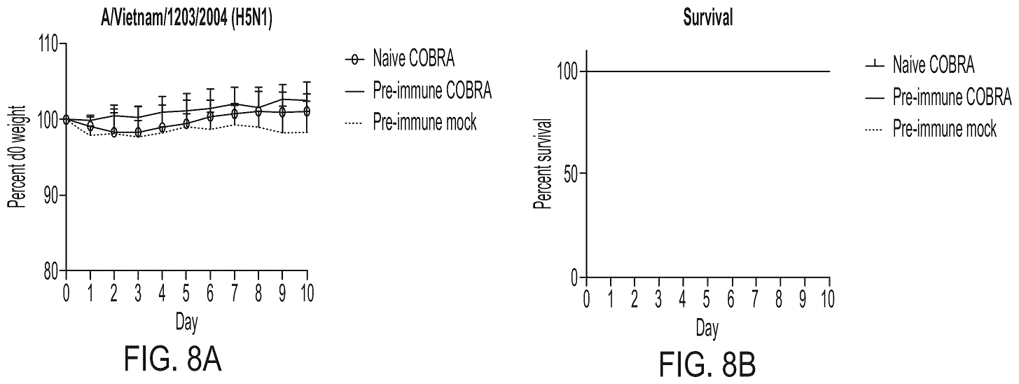

FIGS. 8A and 8B present body weight and survival curves of ferrets in the study after challenge with influenza H5N1 virus. Ferrets were vaccinated twice intranasally at four-week intervals with c-di-AMP as adjuvant. Vaccine groups were pre-immune ferrets immunized with octavalent recombinant influenza immunogenic polypeptides (Cobra antigens), (black line); naïve ferrets immunized with octavalent recombinant influenza immunogenic polypeptides (Cobra antigens), (black line with circle); or pre-immune ferrets given mock immunization/vaccination (grey line). Four weeks after the final immunization/vaccination, ferrets were intranasally infected with a lethal dose of A/Vietnam/1203/2004 (10⁵ PFU) in a volume of 1 mL. The animals were observed for clinical signs and their body weights were recorded daily post infection (FIG. 8A). Survival curve data after infection indicate that all of the animals survived the lethal virus challenge (FIG. 8B). The results show that immunization/vaccination of the ferrets protected the animals from lethal H5N1 virus challenge (10⁵ PFU). H1 pre-immunity also confers protection against H5N1.

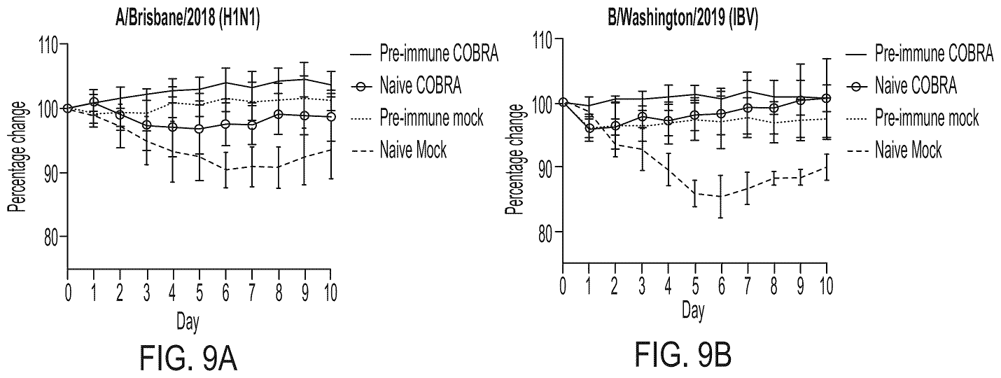

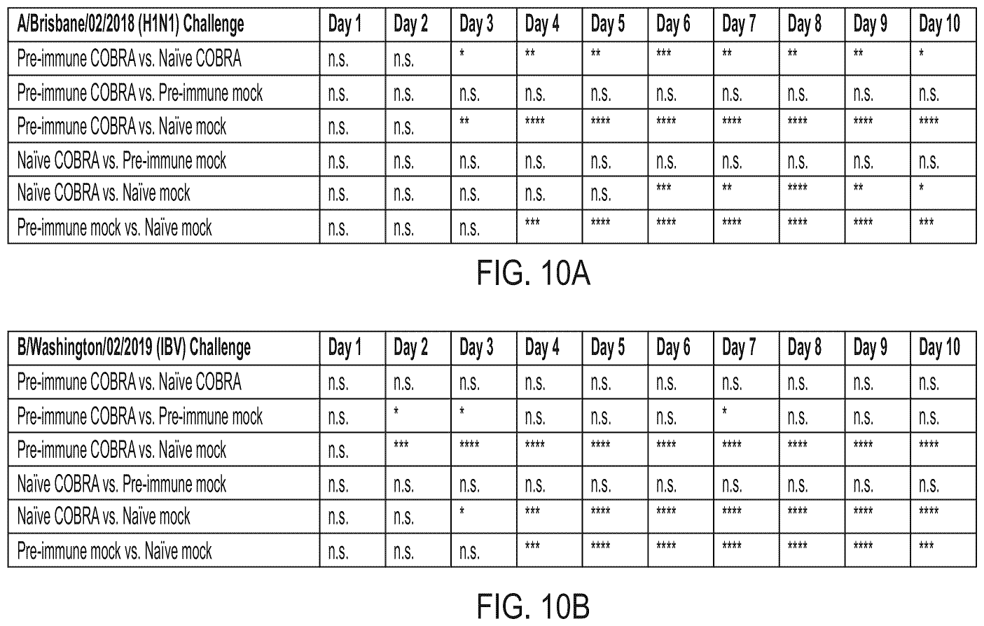

FIGS. 9A and 9B show body weight curves of ferrets after challenges with influenza virus. Ferrets were vaccinated twice intranasally at four-week intervals with c-di-AMP as adjuvant. Vaccine groups were: pre-immune ferrets immunized with octavalent recombinant influenza immunogenic polypeptides (Cobra antigens); naïve ferrets immunized with octavalent recombinant influenza immunogenic polypeptides (Cobra antigens); pre-immune ferrets given mock immunization/vaccination; or naïve ferrets given mock immunization/vaccination. Four weeks after the final vaccination, the ferrets were intranasally infected with influenza virus A/Brisbane/02/2018 (10⁸ PFU), (FIG. 9A), or with influenza virus (b) B/Washington/02/2019 (10⁷ PFU), (FIG. 9B), in a volume of 1 mL. The animals were observed for clinical signs and their body weights were recorded daily post infection. Statistical differences of body weight losses are shown in the tables in FIGS. 10A and 10B.

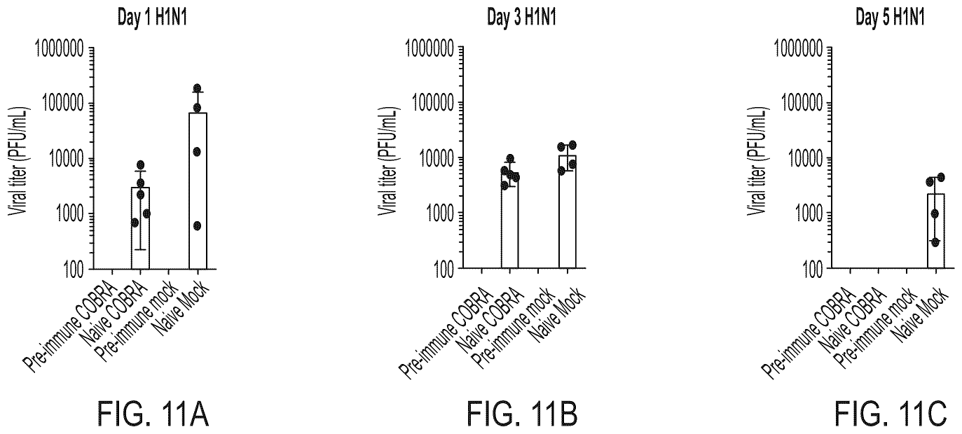

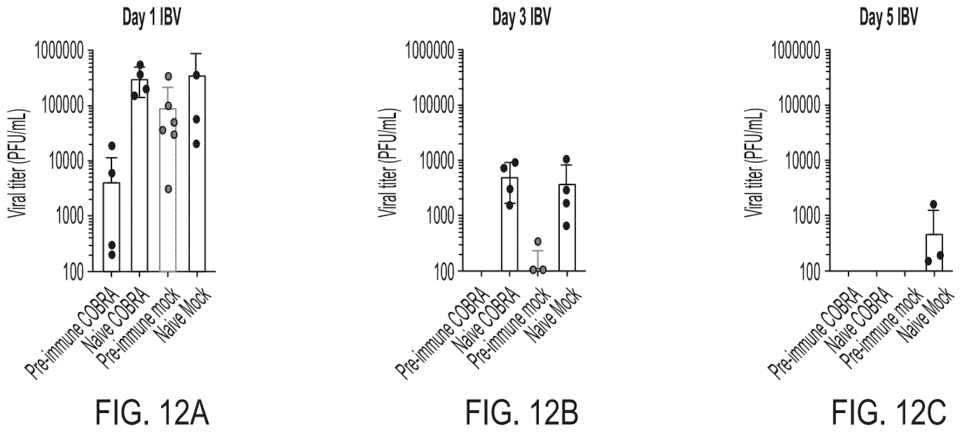

FIGS. 11-11C show graphs of viral titers in upper respiratory tracts of ferrets after infection with influenza virus A/Brisbane/02/2018 (H1N1) and FIGS. 12A-12C show graphs of nasal wash titers of ferrets after infection with influenza virus B/Washington/02/2019 (IBV). Prior to these analyses, ferrets were vaccinated intranasally twice at four-week intervals with c-di-AMP as adjuvant. The groups of immunized animals included pre-immune and naïve ferrets that were immunized with octavalent recombinant influenza immunogenic polypeptides (Cobra antigens) and pre-immune and naïve ferrets that were mock-immunized. Four weeks after the second vaccination, the groups of animals were challenged with H1N1 A/Brisbane/02/2018 virus (FIGS. 11A-11C) or with IBV B/Washington/02/2019 virus (FIGS. 12A-12C). Nasal washes were taken from the animals on day 1, day 3, and day 5 post infection and viral titers were determined. The results demonstrated that by day 5 post infection, no virus was detected in nasal fluids obtained from pre-immune and naïve animals that had been immunized with octavalent recombinant influenza immunogenic polypeptides (Cobra antigens) and in pre-immune animals that had been mock-immunized.

In summary, the results of the ferret studies demonstrated that multivalent recombinant influenza immunogenic polypeptides (Cobra antigens) as described herein elicited a broadly protective immune response against multiple subtypes of influenza viruses. In addition, it was observed that pre-immunity improved the animals' immune response to virus challenge, improved the immune response in animals immunized with recombinant influenza immunogenic polypeptides (Cobra antigens), and ameliorated disease caused by viruses of multiple subtypes.

### Mouse Studies

### Viral Infection and Immunization (Vaccination) of Mice with Broadly-Reactive, Recombinant HA Polypeptide Immunogens

BALB/c and DBA/2J mice (females, 6-8 weeks old) were purchased from The Jackson Laboratory (Bar Harbor, ME, USA). The mice were housed in microisolator units and were allowed free access to food and water. All animals were cared for under the USDA guidelines for laboratory animals, and all procedures were approved by the University of Georgia Institutional Animal Care and Use Committee (IACUC) (no. A2018 06-018-Y3-A16). Seventy-five (75) influenza-naïve BALB/c mice were randomly divided into 15 groups (5 animals/group) and vaccinated intramuscularly with either 3 μg of broadly-reactive, recombinant HA (rHA) immunogenic polypeptides (Cobra antigens) J1, J2, J3, J4, NG1, NG2, or NG3 rHA, as described herein (e.g., J1: SEQ ID NOS: 3, 7, 9); J2: SEQ ID NO: 4); J3 (SEQ ID NO: 5); J4 (SEQ ID NOS: 6, 8); NG1 (SEQ ID NO: 11); NG2 (SEQ ID NOS: 2, 12); NG3 (SEQ ID NO: 13)) with 3 μg of WT rHA (wild type recombinant HA antigens) from historical H3N2 vaccine strains: A/Switzerland/9715293/2013 (Switz/13) (EPI_ISL_162149, MDCK-SP2), A/Hong Kong/4801/2014 (HK/14) (EPI_ISL_259080, MDCK-SP3), A/Singapore/IFNIMH-16-0019/2016 (Sing/16) (EPI_ISL_285898, MDCK-SP3), A/Kansas/14/2017 (Kan/17) (EPI_ISL_403059, MDCK-SP2), A/Switzerland/8020/2017 (Switz/2017) (EPI_ISL_303951, MDCK-SP1), A/South Australia/34/2019 (SA/19) (EPI_ISL_395032, MDCK-SP2); or were administered 25 μL of phosphate-buffered saline (PBS, Corning, Tewksbury, MA, USA) alone as a mock vaccination control.

All immunogens/vaccines (broadly-reactive, influenza immunogenic polypeptide antigens (Cobra antigens), wildtype (WT) rHA antigens, and mock) were formulated with an emulsified squalene-based, oil-in-water emulsion adjuvant, ADDAVAX™ (InvivoGen, San Diego, CA, USA), and the final concentration after mixing 1:1 with rHA was 2.5% squalene. Immunogens/vaccines were administered into the hind leg of the animals on days 0, 28, and 56 in a homologous prime-boost-boost regimen. Blood was collected from the facial vein 14 days following each vaccination, on day 14, 42, and 70. Serum was isolated from the blood of the animals by centrifugation at 2,500 rpm for 10 minutes. Clarified serum was removed and frozen at −20±5° C.

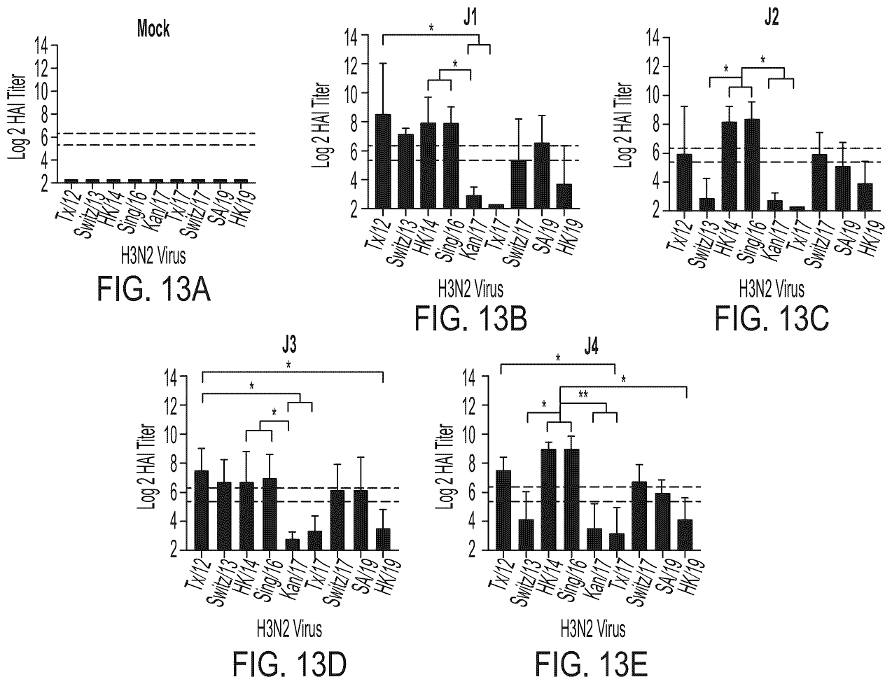

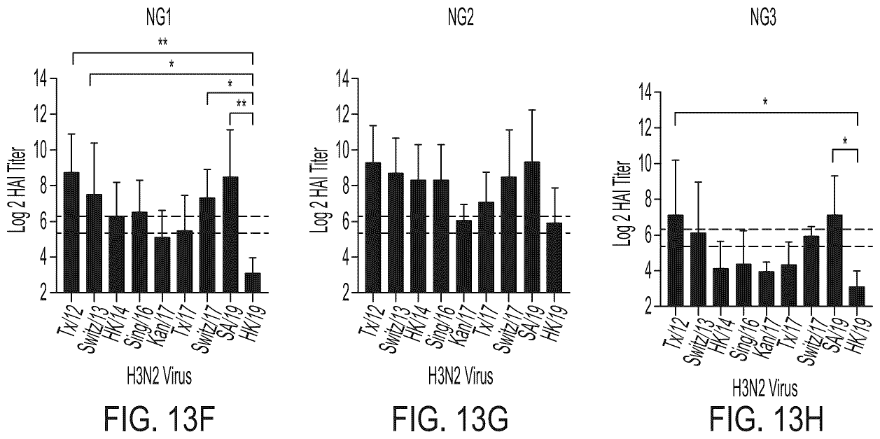

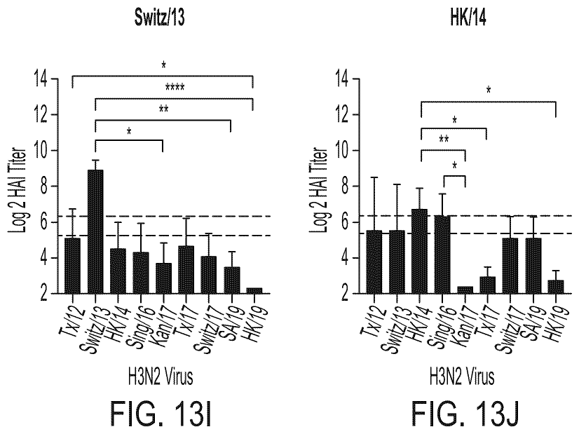

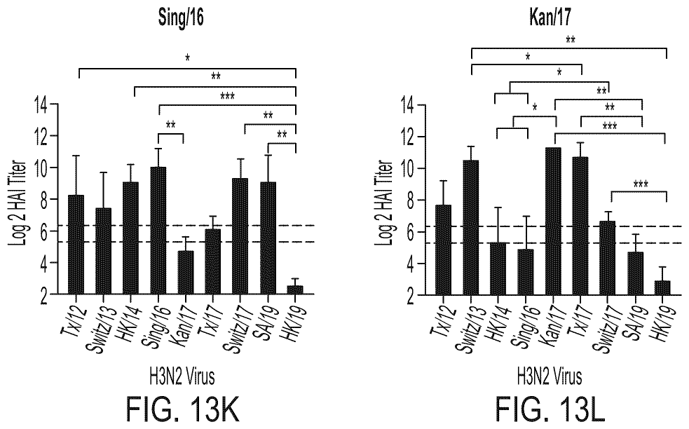

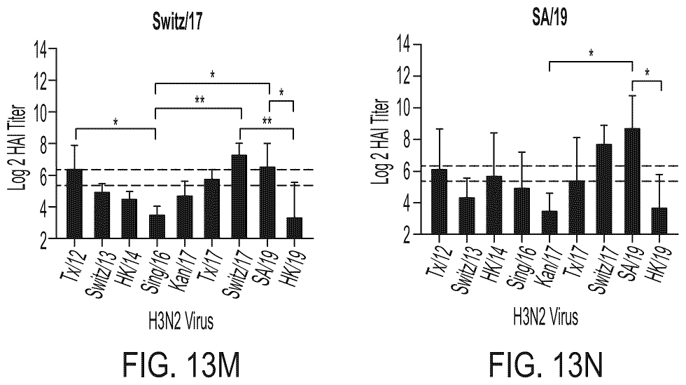

FIGS. 13A-13N show graphs of the results of a hemagglutination inhibition (HAI) assay performed on sera from the 75 influenza-naive BALB/c mice on day 70 post initial vaccination with the recombinant influenza hemagglutinin (rHA) immunogenic polypeptide antigens. The results demonstrated that the mock-vaccinated mice did not have any detectable titers against any of the H3N2 viruses in the panel (FIG. 13A). Mice vaccinated with the broadly-reactive, recombinant influenza immunogenic polypeptide antigen (Cobra antigen (rHA)) J1 (e.g, SEQ ID NOS: 3, 7) had antibodies with HAI activity against the H3N2 influenza viruses isolated from 2012 to 2016 and the Switz/17 and SA/19 strains, but these antibodies failed to recognize the Kan/17, Tx/17, and HK/19 viruses (FIG. 13B). J2 vaccinated mice had antibodies with HAI activity against Tx/12, HK/14, Sing/I 6, and Switz/17 (FIG. 13C). Mice vaccinated with J3 rHA had antibodies with HAI activity against all of the H3N2 influenza virus strains isolated from 2012 to 2016, as well as Switz/17 and SA/19, with their highest antibody titer being directed against the Tx/12 isolate (FIG. 13D). The J4 rHA induced antibodies with HAI activity against Tx/12, HK/14, Sing/I 6, Switz/17, and SA/19 in vaccinated mice, with their highest antibody titers detected against the HK/14 and Sing/16 strains (FIG. 13E). Mice vaccinated with NG1 rHA seroconverted to all of the H3N2 strains isolated from 2012 to 2019, except for Kan/I 7 and HK/19 (FIG. 13F). Mice vaccinated with NG2 rHA possessed seroprotective antibody titers against all of the H3N2 strains in the panel, with their highest antibody titers being directed against the SA/19 virus (FIG. 13G). The NG3 rHA vaccine induced seroconversion to Tx/12, Switz/13. Switz/17, and SA/19 (FIG. 13H). Animals vaccinated with Switz/13 only seroconverted to the homologously matched virus, Switz/13, and to none of the other viruses in the H3N2 panel (FIG. 13I). The HK/14 rHA vaccine induced antibodies with HAT activity against all of the viruses from 2012 to 2016, with its greatest magnitude antibody titers being directed against the homologously matched HK/14 virus. The HK/14 rHA vaccine did not induce antibodies with HAI activity against any of the strains isolated during 2017 to 2019 (FIG. 13J). Mice vaccinated with Sing/16 rHA had antibodies with HAT activity against all of the strains isolated from 2012 to 2019, with the exception of the Kan/17 and HK/19 viruses. The greatest magnitude antibody response for these mice was against the homologously matched Sing/16 virus (FIG. 13K). The Kan/17 rHA vaccine induced anti-bodies with HAT activity against all of the H3N2 isolates from 2012 to 2017, except against the Sing/16 virus. These mice also did not seroconvert to either of the 2019 isolates, SA/19 or HK/19, and the greatest magnitude antibody response from this group was directed toward the homologously matched Kan/17 virus (FIG. 13L). Mice vaccinated with Switz/17 rHA had antibodies with HAI activity against Tx/2, Tx/17, Switz/17, and SA/19; with the greatest magnitude antibody response directed against the homologously matched Switz/17 virus (FIG. 13M). The SA/19 rHA vaccine induced antibodies with HAJ activity against the Tx/12, HK/14, Tx/17, Switz/17, and SA/19 viruses. The highest titer of HAT antibodies for this group was directed against the homologously matched SA/19 virus (FIG. 13N).

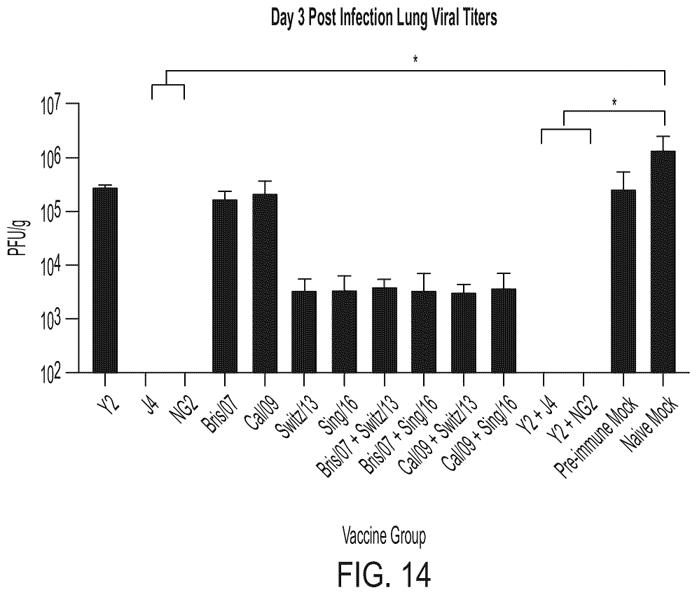

FIG. 14 shows viral titers from mouse lungs collected from mice (n=3 per group) on day 89 (3 days post A/Kansas/14/2017 challenge) to assess the viral load present in the lung tissue. The influenza naïve, mock-vaccinated animals had the most virus present in their lungs. This was significantly higher than preimmune animals vaccinated with either a monovalent J4 HA immunogenic polypeptide antigen or NG2 rHA, as described herein, as well as the bivalent formulations of Y2+J4 or Y2+NG2 rHA, in which there was no detectable virus present in the lung tissue beyond the limit of detection (˜1×10² PFU/g of lung tissue) at day 3 postinfection In contrast, mice vaccinated with monovalent H1 rHAs, Y2, Bris/07, or Cal/09 had lung titers similar to those of the preimmune animals that received mock vaccinations (˜1×10⁵ PFU/g of lung tissue).

One hundred thirty-eight (138)-influenza naïve DBA/2J mice were randomly divided into 17 groups (8 animals/group) to be used in a pre-immune mouse experiment. On day 0, 16 groups of mice were made pre-immune to both HINT and H3N2 influenza viruses by administering a mixture containing equal concentrations of HINT viruses (A/Singapore/6/1986 (Sing/86)) and H3N2 viruses (A/Panama/2007/1999 (Pan/99)) at a final concentration of 5×10⁵ PFU/50 μL in PBS, by administering 50 μL to each mouse intranasally. Mock pre-immune animals were inoculated intranasally with 50 μL of PBS. Following the pre-immune infection, animals were monitored twice daily, morning and evening, for weight loss and clinical signs (labored breathing, lethargy, hunched back, ruffled fur, failure to respond to stimuli, and severe respiratory distress), for 14 days post infection. During this time, none of the mice lost more than 5% of their original body weight, and exhibited no clinical signs. The animals were then allowed to rest for 30 days, at which time they were vaccinated with 3 μg total of either monovalent formulations of broadly-reactive, recombinant HA immunogens (Cobra): Y2 (H1), (e.g., SEQ ID NO: 15); J4 (H3), (e.g., SEQ ID NOS: 6, 8), or NG2 (H3) (e.g., SEQ ID NOS: 2, 12); monovalent formulations of WT rHA: A/Brisbane/59/2007 (Bris/07) (H1), A/California/07/2009 (Cal/09) (H1), A/Switzerland/9715293/2013 (Switz/13) (H3), A/Singapore/IFNIMH-16-0019/2016 (Sing/16) (H3); or 3 μg total (1.5 μg H1+1.5 μg H3) of Cobra rHA polypeptide immunogen cocktails: Y2, Y2+J4 (H1+H3), or Y2+NG2 (H1+H3); or 3 μg total (1.5 μg H1+1.5 μg H3) of WT rHA cocktails: Bris/07+Switz/13 (H1+H3), Bris/07+Sing/16 (H1+H3), Cal/09+Switz/13 (H1+H3), Cal/09+Sing/16 (H1+H3); or mock vaccines containing 25 μL of phosphate-buffered saline (PBS) (Corning, Tewksbury, MA, USA) alone. All vaccines (broadly-reactive, recombinant, polypeptide immunogens (Cobra antigens), WT, and mock) were formulated with ADDAVAX™ adjuvant, and the final concentration after mixing 1:1 with rHA was 2.5% squalene. Vaccines were administered intramuscularly into the hind leg of the animals on days 30 and 58 in a homologous prime-boost regimen. Blood was collected from the facial vein 14 days following the pre-immune infection and each vaccination, on days 14, 44, and 72. Serum was isolated from the blood of animals by centrifugation at 2,500 rpm for 10 minutes. Clarified serum was removed and frozen at −20±5° C. All DBA/2J mice were then challenged intranasally with 50 μL of live H3N2 influenza virus, A/Kansas/14/2107 (EP4), at a concentration of 6.7×10⁶ PFU/50 μL on day 86. Following infection, animals were monitored twice daily, morning and evening, for weight loss and clinical signs (labored breathing, lethargy, hunched back, ruffled fur, failure to respond to stimuli, and severe respiratory distress), for 14 days post infection. On day 89, 3 animals from each group were sacrificed, and the lungs were collected to assess the viral load. Lungs were frozen on dry ice, and stored at −80±5° C. until viral plaque assays were performed.

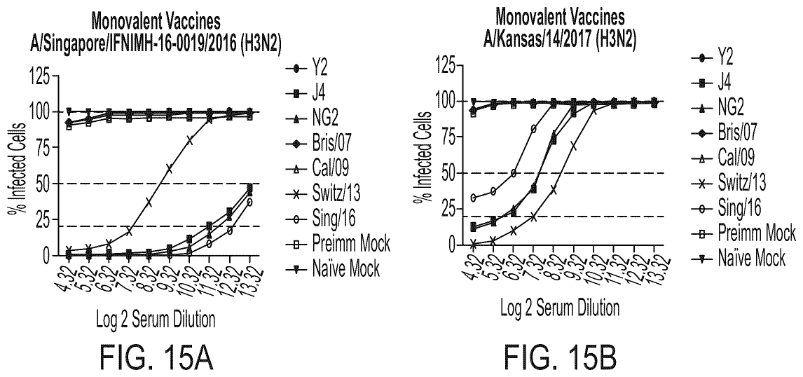

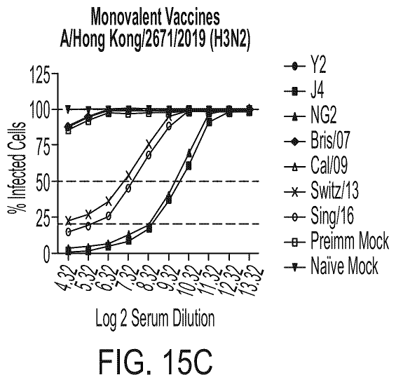

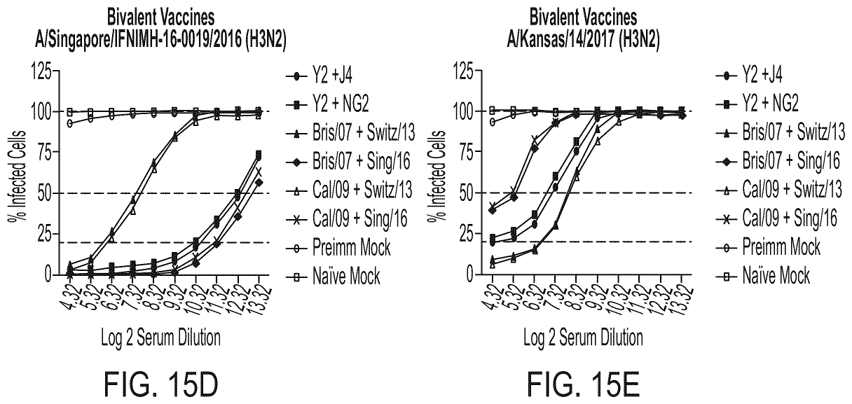

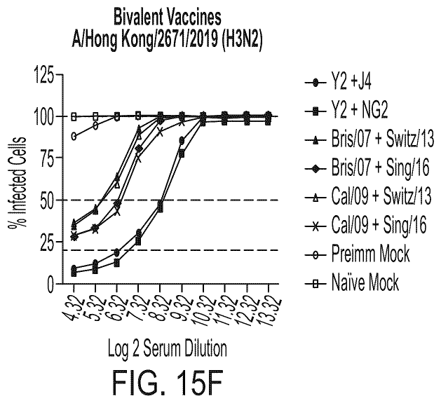

FIGS. 15A-15F show focus reduction assay (FRA) titers of serum from preimmune mice on day 72 to assess the presence of antibodies directed against an H3N2 virus panel. Serum collected from 136 H1+H3 preimmune DBA/2J mice (n=8/group) that were vaccinated with monovalent and bivalent formulations of H1+H3 recombinant influenza immunogenic polypeptides (Cobra antigens) or WT rHA on day 72 post initial infection were pooled for each group and assessed for FRA neutralization, as described below, against the panel of historical H3N2 vaccine strains from 2016-2019. Sera from mice vaccinated with monovalent antigens were tested against the following H1N1 viruses: A/Singapore/IFNIMH-16-0019/2016 (FIG. 15A), A/Kansas/14/2017 (FIG. 15B), and A/Hong Kong/2671/2019 (FIG. 15C). Sera from mice vaccinated with cocktails of bivalent H1+H3 antigens were tested against the following H1N1 viruses: A/Singapore/IFNIMH-16-0019/2016 (FIG. 15D), A/Kansas/14/2017 (FIG. 15E), and A/Hong Kong/2671/2019 (FIG. 15F). The results showed that the broadly reactive, influenza immunogenic polypeptides administered to preimmune animals enhanced the neutralizing antibody responses against recently circulating H3N2 viruses, e.g., Sing/16 viruses.

Preimmune mice vaccinated with the monovalent formulations of the broadly reactive, recombinant HA (rHA) immunogenic polypeptides J4, or NG2 possessed antibodies with high (80%) plaque reduction/neutralization titers (PRNT₈₀ titers) against the Sing/16 virus, which ranged between 10.92 and 11.76 (FIG. 5A). These titers were similar to those generated by the monovalent Sing/16 rHA-vaccinated group, which had an average PRNT₈₀ titer of 12.46 against the homologously matched Sing/16 virus (FIG. 15A). The mice vaccinated with the Switz/13 rHA had ˜6-fold lower neutralization titers than those of the other H3 rHA vaccine antigens, but still produced an average PRNT₈₀ titer of 7.47 against the Sing/16 virus. Mice that received monovalent H1 rHA vaccines, Y2, Bris/07, or Cal/09 had antibodies that neutralized ˜10% of Sing/16 virus infections, similar to those of the preimmune mice that received mock vaccinations (FIG. 15A). The influenza-naive mock-vaccinated mice did not have antibodies that could neutralize Sing/16. (see, e.g., J. D. Allen et al., 2022, J Virology, Vol. 96, No. 7: doi.org/10.1128/jvi.01652-21).

Mice vaccinated with the monovalent J4 or NG2 H3 rHAs generated antibodies with PRNT₅₀ titers against the Kan/17 virus that ranged between 7.56 and 7.82 (FIG. 15B). Mice vaccinated with Switz/13 rHA had the highest level of neutralizing antibodies against Kan/17 with a PRNT₅₀ titer of 8.71 (FIG. 15B). The Sing/16 rHA-vaccinated mice had an average PRNT₅₀ titer of 6.34, which was ˜2-fold lower than those of mice vaccinated with the H3 rHA immunogenic polypeptide antigens and ˜4-fold lower than those of Switz/13 rHA-vaccinated animals (FIG. 15B). The animals that received H1 rHA or mock vaccines had antibodies that neutralized ˜10% of the Kan/17 infections, and influenza-naive animals that were mock vaccinated were unable to neutralize the Kan/17 virus (FIG. 15B). Preimmune mice that were vaccinated with either J4 or NG2 rHA had PRNT₅₀ titers that ranged between 9.64 and 9.89 against the HK/19 v rus, which was the highest neutralizing antibody response against this H3N2 strain (FIG. 15C). Mice vaccinated with Sing/16 had similar PRNT₅₀ titers that ranged between 7.55 and 7.8 against the HK/19 virus, but their titers were ˜4-fold lower than the neutralizing antibody titers generated by J4 and N02 (FIG. 15C). The mice vaccinated with Switz/13 rHA had a slightly lower average PRNT₈₀ titer of 7.12, which was lower than those of any other group of H3 rHA-vaccinated animals (FIG. 15C). Groups that received H1 rHA or mock immunogens/vaccines generated antibodies that neutralized ˜15% of the HK/19 infections (FIG. 15C). Influenza-naive animals that were mock vaccinated were unable to neutralize the 1-1K/19 virus at any serum dilution (FIG. 15C).

Preimmune mice vaccinated with bivalent H1+H3, rHA formulations containing any of the H3 immunogenic polypeptide antigens (Y2+J4, and Y2+NG2) had antibodies with similar neutralizing capabilities against the Sing/16 virus, with average PRNT₅₀ titers that ranged between 12.34 and 1248 (FIG. 15D). These neutralizing antibody titers were similar, but slightly lower, than those elicited by the homologously matched rHA immunogen/vaccine, Sing/16, in which both bivalent formulations, Bris/07+Sing/16 and Cal/09+Sing16, produced PRNT₅₀ titers of 12.75 and 12.99, respectively (FIG. 15D). This is in contrast to mice vaccinated with bivalent combinations containing the Switz/13 rHA, Bris/07+Swxitz/13, and Cal/09+Switz/13, which had ˜10-fold lower PRNT₅₀ titers of 7.49 and 7.74, respectively, against the Sing/I 6 virus (FIG. 15D). However, mice vaccinated with bivalent vaccines containing the Switz/13 rHA, Bris/07+Switz/13, and Cal/09+Switz/13, generated the highest neutralizing antibody titers against the Kan/17 virus, with average PRNT₅₀ titers that ranged between 7.93 and 8.0 (FIG. 15E). Mice vaccinated with bivalent protein formulations containing the Sing/16 H3 rHA, Bris/07+Sing/16, and Cal/09+Sing16, had the lowest average neutralizing antibody titers of any bivalent vaccinations against the Kan/17 virus with PRNT₅₀ titers ranging between 5.21 and 5.45 (FIG. 15E). Aminals vaccinated with rHA formulations containing the H3 immunogenic polypeptide antigens (Y2+J4 and Y2+NG2) had antibodies with average PRNT₅₀ titers between 6.92 and 7.51 against the Kan/17 virus (FIG. 15E). Mice vaccinated with either Y2+J4 or Y2+NG2 generated the highest neutralizing antibody response against the HK/19 virus, with PRNT₈₀ titers of 8.41 and 8.52, respectively (FIG. 15F). Preimmune animals vaccinated with either of the immunogens/vaccines containing the Sing/16 rHA, Bris/07 1 Sing/16, and Cal/09+Sing16, produced similar neutralizing antibody responses against the HK/19 virus, but these titers were ˜4-fold lower than those generated by either Y2+J4 or Y2+NG2 (FIG. 15F). Mice ivaccinated with the Switz/13 rHA (Bris/07+Switz/13 and Cal/09+Switz/13) had the lowest neutralizing antibody titers against the HK/19 virus with identical PRNT₅₀ titers of 5.64 (FIG. 15F).

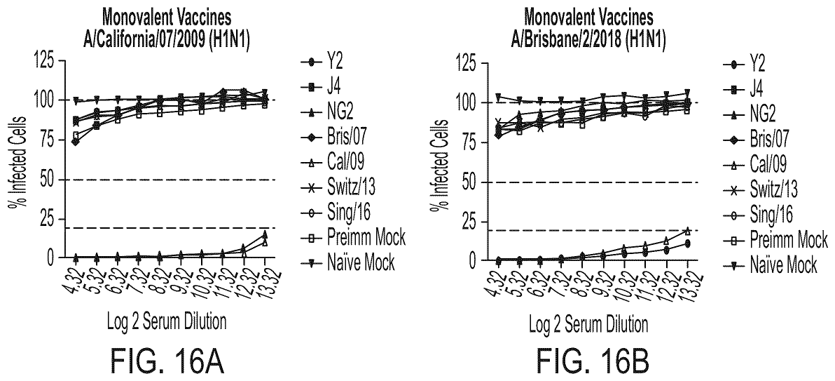

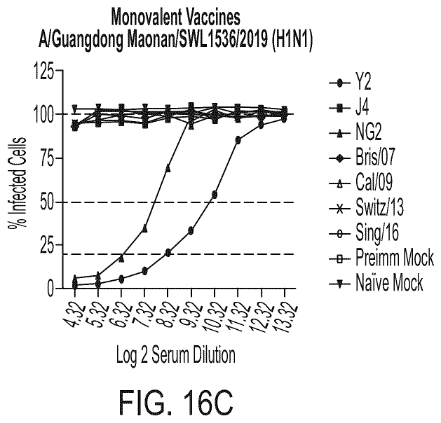

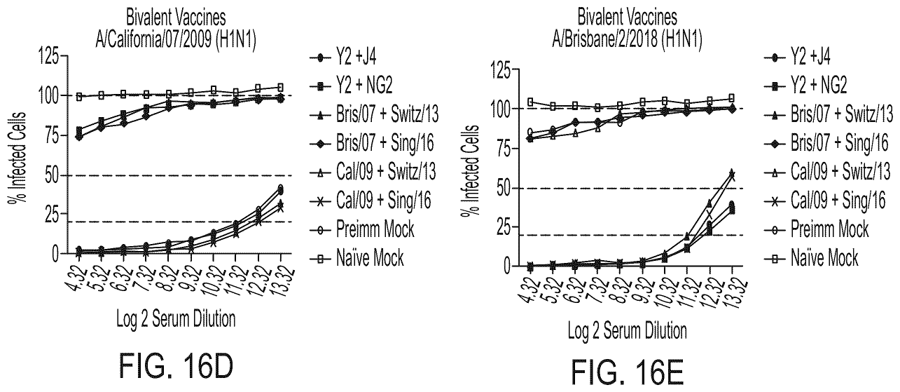

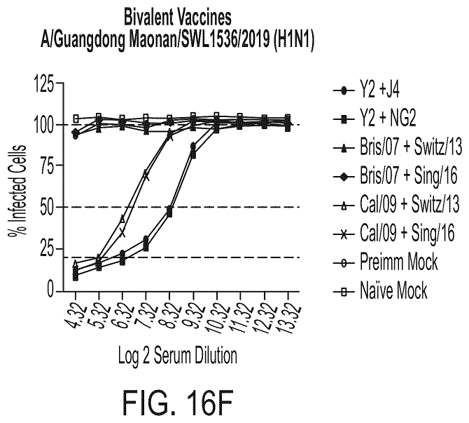

FIGS. 16A-16F show the results of focus reduction assays (FRA) against an H1N1 influenza virus panel carried out using serum from preimmune DBA/2J mice on day 72. The mice (n=8/group) had been vaccinated with monovalent and bivalent formulations of H1+H3 recombinant influenza immunogenic polypeptides (Cobra antigens) or WT rHA. Pooled sera from each group was assessed for FRA neutralization, as described below, against a panel of historical H1N1 vaccine strains from 2009 to 2019. Sera from mice vaccinated with monovalent antigens were tested against the following H1N1 viruses: A/California/07/2009 (FIG. 16A), A/Brisbane/2/2018 (FIG. 16B), and A/Guangdong Maonan/SWL1536/2019 (FIG. 16C). Sera from mice vaccinated with cocktails of bivalent H1+H3 antigens were tested against the following H1N1 viruses: A/California/07/2009 (FIG. 16D), A/Brisbane/2/2018 (FIG. 16E), and A/Guangdong Maonan/SWL1536/2019 (FIG. 16F).

For the study results shown in FIGS. 16A-16F, influenza virus focus reduction assays (FRAs) were used to determine the ability of the broadly reactive, immunogenic polypeptide immunogen-elicited antibodies to neutralize live virus infections against a panel of 3 historical influenza A (H1N1) vaccine strain isolates from 2009 to 2019. Sera used in this assay were collected 72 days after preimmunization from 136 mice (n=8/group), and pooled for each group. Preimmune mice vaccinated with the monovalent formulation of broadly reactive, immunogenic polypeptide immunogen Y2 rHA (e.g., SEQ ID NO: 15) possessed antibodies that neutralized Cal/09 viral infections with an average titer greater than the log₂ 80% neutralization (80% plaque reduction/neutralization titer [PRNT₈₀]) mark at every dilution (FIG. 16A). Similarly, mice vaccinated with Cal/09 rHA antigen also generated antibodies capable of neutralizing Cal/09 viral infection at every dilution tested at a titer greater than the PRNT₈₀ ((FIG. 16A). All other monovalent rHA-vaccinated mice did not generate neutralizing antibodies against the Cal/09 virus antigen, with titers similar to the mock-vaccinated preimmune animals ((FIG. 16A). Sera collected from influenza naive mice that received mock immunizations/vaccinations did not contain antibodies that were able to neutralize the Cal/09 virus antigen at any dilution (FIG. 16A). The preimmune mice vaccinated with the Y2 rHA also generated serum antibodies that neutralized Bris/18 viral infections at a titer of .PRNT₈₀ at every dilution (FIG. 16B). Sera collected from mice vaccinated with Cal/09 rHA antigen also contained antibodies that neutralized Bris/18 viral infection at every dilution with a titer of .PRNT₈₀ (FIG. 16B). All other monovalent rHA immunogen/vaccine groups generated antibodies that prevented fewer than ˜20% of the cells from being infected with Bris/18, similar to the mock-vaccinated preimmune animals, at the lowest serum dilution ((FIG. 16B). Sera collected from influenza naive mice that received mock immunizations/vaccinations did not contain antibodies that were able to neutralize the Bris/18 virus at any serum dilution ((FIG. 168). The preimmune mice vaccinated with Y2 rHA antigen had the highest neutralizing titers of any of the monovalent groups against the Guang/19 virus, with an average log₂ 50% plaque reduction/neutralization titer (PRNT₅₀) titer of 10.14 (FIG. 16C). Mice vaccinated with Cal/09 rHA had serum antibodies that neutralized Guan/19 at an average PRNT₅₀ titer of 7.75; a titer ˜6-fold lower than the titer elicited in Y2 rHA antigen-vaccinated animals (FIG. 16C). All other groups of mice that were vaccinated with monovalent rHA antigens generated serum antibodies that prevented ˜10% of the cells from infections with the Guang/19 virus, similar to the mock-vaccinated preimmune animals (FIG. 16C). Sera from influenza-naive mice that received mock immunizations/vaccinations were unable to neutralize the Guang/19 virus (FIG. 16C).

Mice vaccinated with bivalent rHA formulations containing the Y2 HA immunogenic polypeptide, namely, Y2+J4, and Y2+NG2, all had serum antibodies with similar neutralizing capabilities against the Cal/09 virus, with average PRNT₈₀ titers of 11.08, 11.35, and 11.61, respectively (FIG. 161)). Similarly, preimmune mice vaccinated with mixtures containing the Cal/09 rHA (Cal/09 1 Switz/13 and Cal/09 1 Sing/16) had high PRNT₈₀ titers between 11.96 and 12.32 against the homologously matched Cal/09 virus (FIG. 16D). Mice that were vaccinated with mixtures that contained the Bris/07 H1 rHA (Bris/07 1 Switz/13 and Bris/07 1 Sing/16) prevented ˜25% of the cells from being infected by the Cal/09 virus at the lowest serum dilution, similar to the mock vaccinated preimmune animals (FIG. 16D). Mice that were vaccinated with mixtures containing the Cal/09 rHA (Cal/09 1 Switz/13 and Cal/09 1 Sing/16) also produced high PRNT₈₀ serum titers that ranged between 11.35 and 11.73 against the Bris/18 virus, values which were slightly lower than the values generated against the Cal/09 virus (FIG. 16E). Mice vaccinated with mixtures containing the Y2 rHA antigen (such as Y2+J4 and Y2+NG2) all generated antibodies with similar neutralizing capabilities against the Bris/18 virus, with PRNT₈₀ titers that ranged between 1.75 and 12.13, which were slightly higher than the titers produced by Cal/09 vaccine mixtures (FIG. 16E). Animals vaccinated with mixtures that contained the Bris/07 rHA prevented ˜20% of the cells from being infected with the Birs/18 virus at the lowest serum dilution, similar to the mock vaccinated preimmune animals (FIG. 16E). Mice vaccinated with bivalent formulations containing the Cal/09 rHA antigen (Cal/09 1 Switz/13 and Cal/09 1 Sing/16) produced PRNT₅₀ titers of 6.59 to 6.78, respectively, against the Guang/19 virus (FIG. 16F). In contrast, preimmune animals vaccinated with mixtures containing the COBRA Y2 H1 rHA antigen (such as Y2+J4 and Y2+N02) all generated antibodies with similar neutralizing capabilities against the Guang/19 virus, with PRNT₅₀ titers A4-fold higher than those elicited in the Cal/09 rHA-vaccinated animals with log₂ PRNT50 titers between 8.34 and 8.44, respectively (FIG. 16F). Mice vaccinated with mixtures that contained the Bris/07 rHA prevented ˜10% of the cells from being infected with Guang/19 virus at the lowest serum dilution, similar to the mock-vaccinated preimmune animals (FIG. 16E). Average HAI and PRNT₅₀ titers were also compared for each group. In general, an HAI log₂ geometric mean titer (GMT) of ˜5.5 or higher correlated with a log 2 PRNT₅₀ titer of ˜8.34 or higher, indicating that an HAI titer correlated with 50% protection in humans (5.32) can be a predictive measure of neutralization observed in FRAs against influenza A(H1N1) viruses. The results demonstrated that COBRA vaccines enhanced neutralizing antibody responses against modern pandemic-like H1N1 viruses.

### Materials, Reagents and Methods Used in Example 2

### Viruses and HA Antigens

Influenza A(H3N2) viruses were obtained through either the Influenza Reagents Resource (IRR), BEI Resources, the Centers for Disease Control (CDC), or provided by Virapur (San Diego, CA, USA). Viruses were passaged once in the same growth conditions as they were received, i.e., either embryonated chicken eggs or semi-confluent Madin-Darby canine kidney (MDCK) cell cultures as per the instructions provided by the WHO. H3N2 virus lots were titered with 0.75% guinea pig erythrocytes in the presence of 20 nM Oseltamivir, and made into aliquots for single-use applications. H1N1 virus lots were titered with 0.8% turkey erythrocytes, and made into aliquots for single use applications.

The A(H3N2) 2012-2019 historical influenza vaccine strain viral panel for HAI analysis included the following eight (8) viral strains: A/Texas/50/2012 (Tx/12) egg passage 4 (EP4) (clade 3c2), A/Switzerland/9715293/2013 (Switz/13) EP4 (clade 3c3.a), A/Hong Kong/4801/2014 (HK/14) EP11 (clade 3c2.a), and A/Singapore/IFNIMH-16-0019/2016 (Sing/16) EP3 (clade 3c2.al), A/Kansas/14/2017 (Kan/17) EPI (clade 3c3.a), A/Texas/71/2017 (Tx/17) MDCK-siat cell passage 1 (MDCK-SP1) (clade 3c3.a), A/Switzerland/8060/2017 (Switz/17) EPI (clade 3c3.a2), A/South Australia/34/2019 (SA/19) EPI (clade 3c2.alb/131K), A/Hong Kong/2671/2019 (HK/19) EPI (clade 3c2.alb/137F).

The A(H1N1) 2007-2019 historical influenza vaccine strain panel for HAI analysis included the following five (5) Influenza A virus (IAV) strains: A/Brisbane/59/2007 (Bris/07) EP1, A/California/07/2009 (Cal/09) EP4, A/Michigan/45/2015 (Mich/15) EP1, A/Brisbane/02/2018 (Bris/18) EP1, and A/Guangdong-Maonan/SWL1536/2019 (Guang/19) EP1. For H1N1 HAI analysis in ferrets, A/Solomon Islands/03/2006 (Genbank Accession No. EU100724) was also used.

The IBV 2006-2019 panel for HAI analysis included the following six (6) viral strains: for Yamagata-like lineages B/Florida/04/2006 (Genbank Accession No. KF009552), B/Massachusetts/02/2012 (Genbank Accession No. C892118), B/Phuket/3073/2013 (NCBI Accession No. EPI1799823); for Victoria-like lineages B/Brisbane/60/2008 (Genbank Accession No. FJ766840), B/Colordado/06/2017 (Genbank Accession No. CY232066), and B/Washington/02/2019 (Genbank Accession No. MK676295).

The H5 2004-2014 panel for HAI analysis included the following six (6) viral strains: A/Vietnam/1203/2004 (H5N1, Genbank Accession No. AAW80717.1), A/whooper swan/Mongolia/244/2005 (H5N1, Genbank Accession No. ACD68156.1), A/Egypt/321/2007 (H5N1, Genbank Accession No. AEL31632.1), A/Hubei/01/2010 (H5N1, Genbank Accession No. AEO89181.1), A/Guizhou/01/2013 (H5N1, Genbank Accession No EPI420386). A/Sichuan/26221/2014 (H5N6, Genbank Accession No. EPI533583).

One historical H1N1 vaccine strain virus, A/Singapore/6/1986 (Sing/86) EP1, and one historical H3N2 vaccine strain virus, A/Panama/2007/1999 (Pan/99) EP4, were used to make mice pre-immune to H1N1 and H3N2 viruses. The pre-immune mice were also challenged with an H3N2 influenza virus, A/Kansas/14/2017 (Kan/17) EP1 on day 86 of the pre-immune study.

For ferrets, CA/09, Pan/99 and B/Hong Kong/330/2001 virus strains (Genbank Accession No. AF532549) were used to establish pre-immunity against all 3 seasonal subtypes of viruses. Ferrets were challenged with Bris/18 (H1N1), B/WA/19 (IBV), or A/Vietnam/1203/2004 (Vn/04).

### Hemagglutination-Inhibition (HAI) Assay

The hemagglutination inhibition (HAI) assay was used to assess the presence of functional anti-hemagglutinin (HA) antibodies (e.g., in serum obtained from bled animals) that are able to inhibit agglutination of guinea pig erythrocytes for H3N2 viruses, and turkey erythrocytes for H1N1 viruses. The protocols were adapted from the WHO laboratory influenza surveillance manual. (See, J. D. Allen et al., 2022, J Virology, Vol. 96, No. 7: doi.org/10.1128/jvi.01652-21). Guinea pig red blood cells are frequently used to characterize contemporary A(H3N2) influenza strains that have developed a preferential binding to alpha (2,6) linked sialic acid receptors. To inactivate nonspecific inhibitors, serum samples were treated with receptor-destroying enzyme (RDE) (Denka Seiken, Co., Japan) prior to being tested. Briefly, three parts of RDE was added to one part of serum and the samples were incubated overnight at 37° C. RDE was inactivated by incubation at 56° C. for 30 minutes.

RDE-treated sera were diluted in a series of two-fold serial dilutions in v-bottom microtiter plates. An equal volume of each A(H3N2) virus, adjusted to approximately 8 hemagglutination units (HAU)/50 μl in the presence of 20 nM Oseltamivir carboxylate, was added to each well. The plates were covered and incubated at room temperature for 30 minutes, and then 0.75% guinea pig erythrocytes (Lampire Biologicals, Pipersville, PA, USA) in PBS were added. Prior to use, the red blood cells (RBCs) were washed twice with PBS, stored at 4° C., and used within 24 hours (h) of preparation. The plates were mixed by gentle agitation, covered, and the RBCs were allowed to settle for 1 h at room temperature. The HAI titer was determined by the reciprocal dilution of the last well that contained non-agglutinated RBCs. Positive and negative serum controls were included for each plate.

In separate assays, RDE-treated sera were diluted in a series of two-fold serial dilutions in v-bottom microtiter plates. An equal volume of each A(H1N1) virus, adjusted to approximately 8 hemagglutination units (HAU)/50 μl, was added to each well. The plates were covered and incubated at room temperature for 20 mins, and then 0.8% turkey erythrocytes (Lampire Biologicals, Pipersville, PA, USA) in PBS were added. Prior to use, the RBCs were washed twice with PBS, stored at 4° C., and used within 24 h of preparation. The plates were mixed by gentle agitation, covered, and the RBCs were allowed to settle for 30 mins at room temperature. The HAI titer was determined by the reciprocal dilution of the last well that contained non-agglutinated RBCs. Positive and negative serum controls were included for each plate.

All mice were negative (HAI≤1:10) for pre-existing antibodies to human influenza viruses prior to infection or vaccination, and for the animal studies described supra, a positive HAI reaction (HAI+), or “sero-protection,” is defined as an HAI titer≥1:40, while “seroconversion” refers to a 4-fold increase in titer compared to baseline, as per the WHO and European Committee for Medicinal Products to evaluate influenza vaccines.

### Focus Reduction Assay (FRA)

The Focus Reduction Assay (FRA) used in the animal studies was initially developed by the WHO collaborating Centre in London, U.K. and then was modified by U.S. Centers for Disease Control and Prevention (CDC). MDCK-SIAT1 cells (Sigma, St. Louis, MO, USA) were plated at 2.5-3×10⁵ cells/ml (100 μL/well in a 96-well plate) one day prior to use in the assay. Cells were cultured in Dulbecco's Modified Eagle Medium (DMEM) containing 5% heat-inactivated fetal bovine serum and antibiotics in 96-well flat bottom plates overnight to form a 95-100% confluent monolayer. The following day, the cell monolayers were rinsed with 0.01 M phosphate-buffered saline (PBS) at pH 7.2 (Gibco, Waltham, MA, USA), followed by the addition of 2-fold serially diluted RDE treated serum (50 μL per well) starting with a 1:20 dilution in virus growth medium containing TPCK-treated trypsin (1 μg/ml) (Thermo Fisher, Waltham, MA, USA), VGM-T, (i.e., DMEM containing 0.1% BSA, 1% Penicillin/Streptomycin (100 U/mL Penicillin, 100 μg/mL Streptomycin solution), and 1 μg/ml TPCK-treated trypsin) (Sigma, St. Louis, MO, USA). 50 μL of A(H3N2) influenza virus (1.2×10⁴ focus forming units (FFU)/mL, which corresponds to 600 FFU/50 μl) in VGM-T was added to the wells of each plate. VGM-T only was added to the wells containing the control wells. Virus stocks were standardized by previous titration in the FRA. The A(H3N2) viruses used in the assay were the historical WHO-selected vaccine strains A/Singapore/IFNIMH-16-0019/2016 EP3, A/Kansas/14/2017 EP1, and A/Hong Kong/2671/2019 EP1. The A(H1N1) viruses used in the assay were the historical WHO-selected vaccine strains A/California/07/2009 EP4, A/Brisbane/2/2018 EP1, and A/Guangdong-Maonan/SWL1536/2019 EP1.

Following a 2-hour incubation period at 37° C. in 5% CO₂, the cells in each well were then overlaid with 100 μL of equal volumes of 1.2% Avicel RC/CL (Type: RC581 NF; FMC Health and Nutrition, Philadelphia, PA, USA) in 2× Modified Eagle Medium containing 1 μg/ml TPCK-treated trypsin, 0.1% BSA and antibiotics. Plates were incubated for 18-22 h at 37° C., 5% CO₂. The overlays were then removed from each well, and the monolayer was washed once with PBS to remove any residual Avicel. The plates were then fixed with ice-cold 4% formalin in PBS for 30 min at 4° C., followed by a PBS wash and permeabilization using 0.5% Triton-X-100 in PBS/glycine at room temperature (RT) for 20 min. Plates were washed three times with wash buffer (PBS, 0.1% TWEEN-20; PBST) and then incubated for 1 h with a monoclonal antibody directed against influenza A nucleoprotein obtained from the Influenza Reagent Resource (IRR) (Manassas, VA, USA) (FR-1217) (1 mg/mL), diluted 1:2000 in ELISA buffer (PBS, 10% horse serum, 0.1% TWEEN-80). Following washing (3× PBST), the cells were incubated with goat anti-mouse peroxidase-labelled IgG (Sera Care, Inc., Milford, MA, USA) (KPL 474-1802) (1 mg/mL), diluted 1:2000 in ELISA buffer for 1 hour at RT. Plates were then washed again (3× PBST) and infectious virus foci were visualized using TrueBlue substrate (Sera Care, Inc., Milford, MA USA) containing 0.03% H₂O₂ incubated at RT for 10 min. The reaction was stopped by washing five times with dH₂0. Plates were air-dried and foci were enumerated using a CTL BioSpot Analyser with ImmunoCapture 6.4.87 software (CTL, Shaker Heights, OH, USA). The FRA titer was reported as the reciprocal of the highest dilution of serum corresponding to 50% foci reduction compared to the virus control minus the cell control.

In order for a plate to pass quality control (QC), both the average of the octuplet virus control wells (VC), as well as the average of the octuplet cell control wells (CC) must also pass QC. The virus controls must fall between 200 and 1600 foci and the cell controls must be free of foci. Additionally, the positive control, A(H1N1) and A(H3N2) historical influenza vaccine strain viruses were run in triplicate plates in each individual assay and at least two out of three plates must pass VC and CC criteria. Homologous mouse antisera, previously generated through infection with homologously-matched A(H1N1) and A(H3N2) influenza viruses at 1×10⁶ FFU/mL, and collected 14 days post infection, must have the same titer across the plates. Each assay plate (one virus per plate) contained a panel of mouse reference antisera, as well as a human influenza vaccine serum control to assess overall assay consistency. The percentage of infected cells reported in the assay was calculated by averaging the foci count from the positive control (virus and cell only) wells, and dividing the number of foci in each experimental well by the average of the positive control.

### Influenza Viral Plaque Assay

MDCK cells (Sigma, St. Louis, MO, USA) were seeded into each well of a six-well plate at a concentration of 1×10⁶ cells/well one day prior to performing the plaque assay. On the day of the assay, frozen lung tissues were thawed on ice and were weighed and homogenized in 1 ml of DMEM (Thermo Fisher, Waltham, MA, USA). The homogenate was centrifuged at 2,000 rpm for 5 min to remove tissue debris. The supernatant was collected and subjected to a serial 10-fold dilution in DMEM supplemented with 1% penicillin-streptomycin (DMEM+P/S) (Thermo Fisher, Waltham, MA, USA). When MDCK cells reached 90% confluency in each well, the plates were washed 2× with DMEM+P/S, and infected with 100 μL of each dilution of homogenate supernatant. The plates were then shaken every 15 minutes for 1 hour. After 1 hour of incubation, the supernatant was removed and cells were washed twice with fresh DMEM+P/S. Following the second wash, a solution of 2×MEM and 1.6% agarose (Thermo Fisher, Waltham, MA, USA) mixed 50:50 v/v, and supplemented with 1 μg/mL of TPCK trypsin (Thermo Fisher, Waltham, MA, USA) was added into each well. Plates were then incubated at 37° C.+5% CO₂ for 72 hours. After 72 hours, the gel overlays were removed from each well, and the cells were fixed with 10% buffered formalin for 10 minutes and stained with 1% crystal violet (Thermo Fisher, Waltham, MA, USA) for 10 minutes at RT. Plates were then rinsed thoroughly 5 times with fresh water to remove excess crystal violet. Plates were allowed to air dry for 24 hours, and the viral plaques were enumerated as the reciprocal of each dilution. The lung viral titers were calculated and presented as plaque forming units (PFU)/g of lung tissue.

Similarly, for the ferret nasal wash plaque assay, nasal wash samples were thawed on the day of assay and serially diluted 10-fold in DMEM+P/S. 100 μL of each dilution were placed on 90% confluent MDCK cells that had been washed 2× with DMEM+P/S. The plates were shaken every 15 minutes for 1 hour as mentioned above. The plates were washed and were incubated at 37° C.+5% CO₂ for 72 hours in the agarose overlay. Afterward, the overlays were removed, and the cells were fixed, stained, and enumerated as described above. Viral titers were calculated and presented as PFU/mL of nasal wash sample.

### Enzyme-Linked Immunosorbent Assay (ELISA)

ELISA was used to assess antibody reactivity against different H1N1 HA strains and performed as previously described. In brief, Immulon 4HBX plates (Thermo Fisher Scientific, Waltham, MA, USA) were coated at 4° C. overnight with 50 μl per well with a solution of carbonate buffer (pH 9.4) containing 1 μg/mL of the different rHAs (A/California/07/2009, A/Brisbane/02/2018), or cH6/1 purified rHA and 5 μg/ml of bovine serum albumin (BSA) in a humidified chamber. 5 μg/ml BSA (50 μl per well) were coated alone as a negative control. Plates were blocked with ELISA blocking buffer in a volume of 200 μl/well for 1 hour at 37° C. Serum samples were serially diluted 3-fold in blocking buffer starting from a dilution of 1:100, and then added to HA protein coated plates. After incubation at 4° C. overnight, the 1:2000 diluted goat anti-mouse IgG (Southern Biotech, Birmingham, AL, USA) secondary antibody was added to each well in a volume of 100 μl, and incubated at 37° C. for 1 hour. Finally, 50 μl of ABTS substrate (VWR Corporation) was added to each well, and the plate was further incubated at 37° C. for 15-20 mins. Colorimetric conversion was terminated by adding 50 μl of 1% SDS to each well. The O.D. values (OD 414) were measured by a spectrophotometer (PowerWave XS, BioTek) at 414 nm.

Similarly, ELISA was used to assess antibody reactivity in ferret serum against influenza HA or NA polypeptide antigens (Cobra antigens). Plates were coated at 4° C. overnight with 50 μl per well in carbonate buffer containing 1 μg/mL of the different rHAs and rNAs in a humidified chamber. Plates were blocked with ELISA blocking buffer in a volume of 200 μl/well for 1 hour at 37° C. Serum samples were serially diluted 3-fold in blocking buffer starting from a dilution of 1:100, and then added into the protein coated plates. After incubation at 4° C. overnight, plates were washed 5 times in washing buffer (0.05% TWEEN-20 in PBS). 100 μl of goat anti-ferret IgG H&L HRP (Abcam, Boston, MA) diluted at 1:4000 in blocking buffer was added, and the plates were incubated for 1 hour at 37° C. The plates were washed 5 times in washing buffer. 50 μl of ABTS substrate (VWR, Radnor, PA) was added to each well, and the plate was further incubated at 37° C. for 15-20 mins. Colorimetric conversion was terminated by adding 50 μl of 1% SDS into each well. The O.D. values (OD 414) were measured by a spectrophotometer (PowerWave XS, BioTek) at 414 nm.

### Statistical Analysis

Data is presented as absolute mean values ±standard error of the mean (SEM). One-way ANOVA and Two-way ANOVA with multiple comparisons were used to analyze the statistical differences between groups using GraphPad Prism 9 software (GraphPad, San Diego, CA, USA). A “p” value less than 0.05 was defined as statistically significant (*, P<0.05; **, P<0.01; ***, P<0.001; ****, P<0.0001).

### Example 3: Mouse Studies Using Recombinant Influenza Virus HA Immunogenic Peptides (Influenza H1N1 Viruses) as Immunogens (Vaccines)

A study similar to those described in Example 2 was performed in vivo in pre-immune and naïve mice using recombinant influenza virus HA immunogenic polypeptides, (influenza H1N1 viruses), as immunogens (vaccines) to determine the immunogenicity and efficacy of the recombinant influenza HA immunogenic polypeptides in eliciting broadly protective immune responses against seasonal and pandemic influenza viruses using an in vivo mouse model. As described in Examples 1 and 2, the broadly reactive, non-naturally occurring, HA polypeptide immunogen(s) (and recombinant HA polypeptide immunogen(s)) constitute influenza virus Hemagglutinin (HA) antigen amino acid sequences derived from influenza H1 types, such as H1N1, e.g., containing HA sequences representing those of seasonal or pandemic influenza viruses.

H1N1 Influenza viruses used in this study included: A/Chile/1/1983 (GenBank Accession No. CY121261.1), A/Singapore/6/1986 (GenBank Accession No. CY020477.1), A/Beijing/262/1995 (GenBank Accession No. CY033614.1), A/New Caledonia/20/1999 (GenBank Accession No. EF566076.1), A/California/07/2009 (GISAID Accession No. EPI516535), A/Brisbane/02/2018 (GISAID Accession No. EPI1799929), and A/Guangdong-Maonan/SWL 1536/2019 (GISAID Accession No. EPI1921671). All viruses were obtained from Virapur (NY) or BEI Resources. Each virus was amplified in embryonated chicken eggs.

### Animal Immunization/Vaccination and Infection

BALB/c and DBA/2J mice (females, 6 to 8 weeks old) were purchased from Jackson Laboratory (Bar Harbor, ME, USA), housed in microisolator units, and allowed free access to food and water. The animals were cared for under USDA guidelines for laboratory animals. All procedures were reviewed and approved by the University of Georgia Institutional Animal Care and Use Committee (IACUC) (no. A2018 06-018-Y3-A16). Eighty-eight (88) BALB/c mice were randomly divided into 8 groups, with 11 mice in each group. Mice were vaccinated intramuscularly with either 1 μg of recombinantly produced, non-naturally occurring, broadly reactive H1 HA polypeptide immunogen (Cobra); Brisbane/59/2007, California/07/2009, Brisbane/02/2018 virus-like particles (VLPs); or PBS formulated with ADDAVAX™ (oil-in-water emulsion) (InvovoGen, San Diego, CA, USA) at a 1:1 ratio for a final volume of 50 μL. At weeks 4 and 8 following the first vaccination, mice were boosted intramuscularly with the same amount of VLPs or PBS. (Y. Huang et al., 2021, Vaccines, 9(7):793, the contents of which are incorporated herein by reference).

Another group of 40 DBA/2J mice were divided into 5 groups (n=8/group) and were vaccinated with 1 μg of the corresponding soluble recombinant HA proteins mentioned above using the same vaccination regimen. Blood was collected at weeks 6 and 10 following the first vaccination and sera were separated and stored at −20° C. for future use. At week 12, all mice were infected intranasally with 5×10⁴ PFU of wild-type H1N1 A/California/07/2009 (CA/09) or 8.75×10⁶ PFU of H1N1 A/Brisbane/02/2018 (Bris/18) in a volume of 50 μL. The animals were monitored and their body weights were recorded daily for 14 days post infection. At days 3 and 6 post infection, three mice from each group were sacrificed and the lungs were collected. The left lung was inflated with 10% neutral formalin for histopathology, and the right lung lobes were snap-frozen on dry ice and then stored at −80° C. for assessing virus titers. Mice were humanely euthanized once they reached humane endpoints by losing 20% of their original body weight or accumulated a clinical disease score of 3. All procedures were performed in accordance with Guide for the Care and Use of Laboratory Animals, the Animal Welfare Act, and Biosafety in Microbiological and Biomedical Laboratories.

An ELISA was used to assess antibody reactivity against different H1N1 HA strains and performed as described in G. A. Kirchenbaum G. A. et al., (2017, J Immunol.; 199:3798-3807). In brief, Immulon 4HBX plates (Thermo Fisher Scientific, Waltham, MA, USA) were coated at 4° C. overnight at 50 μL per well with a solution of carbonate buffer (pH 9.4) containing 1 μg/mL of the different rHAs (A/California/07/2009, A/Brisbane/02/2018) and 5 μg/mL of bovine serum albumin (BSA) in a humidified chamber. An amount of 5 μg/mL BSA (50 μL per well) was coated alone as a negative control. Plates were blocked with ELISA blocking buffer in a volume of 200 μL/well for 1 h at 37° C. Serum samples were serially diluted 3-fold in blocking buffer starting from a dilution of 1:100, and then added into HA protein coated plates. After incubation at 4° C. overnight, goat anti-mouse IgG (Southern Biotech, Birmingham, AL, USA) secondary antibody (diluted 1:2000) was added into each well in a volume of 100 μL, and the plates were incubated at 37° C. for 1 h. Finally, 50 μL of ABTS substrate (VWR Corporation) was added into each well, and the plates were further incubated at 37° C. for 15-20 min. Colorimetric conversion was terminated by adding 50 μL of 1% SDS into each well. The O.D. values (OD 414) were measured at 414 nm using a spectrophotometer (PowerWave XS, BioTek).

A plaque assay was performed to analyze the samples. For the plaque assay, MDCK cells within 20 passages were seeded in each well of a six-well plate at a concentration of 1×10⁶ cells/well one day prior to performing the plaque assay. Frozen lung tissues were thawed on ice and homogenized in 1 mL of DMEM. The homogenate was centrifuged at 2000 rpm for 5 min to remove tissue debris, and the supernatant was collected and subjected to a serial 10-fold dilution in DMEM supplemented with 1% penicillin-streptomycin. MDCK cells having 90% confluency in each well were infected with 100 μL of each dilution of homogenate supernatant. The plates were then shaken every 15 min for 1 h. After the 1 h incubation, the supernatant was removed, and the cells were washed twice with fresh DMEM. Finally, 2 mL of 2× MEM and 0.8% agarose overlay (Cambrex, East Rutherford, NJ, USA) were added into each well, and the plates were incubated at 37° C. with 5% CO₂ for another 72 h. Thereafter, the overlay was removed from each well, and the cells were fixed with 10% buffered formalin for 20 min and stained with 1% crystal violet (Fisher Science Education, Waltham, MA, USA) for 15 min at room temperature (RT). Plates were then rinsed thoroughly using tap water to remove excess crystal violet. The plaques were enumerated, and the lung viral titers were calculated and presented as PFU/mL.

Hemagglutination-Inhibition (HAI) assays were used to evaluate the presence of functional antibodies that bound to HA protein and that were capable of inhibiting red blood cell agglutination. The protocol was adapted from the WHO laboratory influenza surveillance manual (WHO Global Influenza Surveillance Network. Manual for the Laboratory Diagnosis and Virological Surveillance of Influenza. World Health Organization; Geneva, Switzerland: 2011). In this study, HAI assays were performed against a panel of 7 H1N1 influenza viruses, including: A/Chile/i/1983, A/Singapore/6/1986, A/Beijing/262/1995, A/New Caledonia/20/1999, A/California/07/2009, A/Brisbane/02/2018, and A/Guangdong-Maonan/SWL 1536/2019. The HAI assays were performed as described by D. M. Carter et al. (2016, J. Virol. 2016; 90: 4720-4734). Briefly, sera were treated with receptor-destroying enzyme (RDE) (Denka Seiken, Co., Tokyo, Japan) prior to being tested to remove nonspecific inhibitors by incubating overnight at 37° C. The RDE was then further inactivated at 56 C for 45 min. An amount of 25 μL of PBS was added to a 96-well V-bottom plate in rows 2-12; 50 μL RDE-treated sera was added into row 1; and then a 2-fold serial dilution was performed across the plate. An equal volume of H1N1 virus with approximately 8 hemagglutination units (HAU)/50 μL was added into each well. The plates were incubated at RT for 30 min, and then a solution of 0.8% turkey erythrocytes in PBS were added in a volume of 50 μL to each well. The plate was mixed by agitation and incubated at RT for another 30 min. The HAI titer was determined as the reciprocal dilution of the last well that contained non-agglutinated RBCs. Positive and negative serum controls were included for each plate. An HAI titer greater than 1:40 was defined as seroprotective, and a 4-fold increase in HAI titer compared to the baseline was considered seroconversion in accordance with the WHO and European Committee for Medicinal Products guidelines to evaluate influenza vaccines (European Medicines Agency, 2014, Guideline on Influenza Vaccines: Non-Clinical and Clinical Module (Draft) European Medicines Agency; London, UK).

A Focus Reduction Assay (FRA) initially developed by the World Health Organization collaborating center in London, UK and modified by U.S. Centers for Disease Control and Prevention (CDC) was used in this study. Briefly, serum samples were treated with RDE as described above. Initially, 100 μL of MDCK cells at a concentration of 3×10⁵ cells/mL were seeded into each well in a 96-well flat-bottom plate. After 24 h, the cells were allowed to reach 95% to 100% confluence and were then washed with PBS. Next, 50 μL of 2-fold serial diluted serum samples were added to each well starting with a 1:20 dilution in virus growth medium (VGM) supplemented with 1 μg/mL tosylsulfonyl phenylalanyl chloromethyl ketone (TPCK)-treated trypsin (VGM-T) (Sigma, St. Louis, MO, USA). Afterward, influenza virus was diluted in VGM-T, and 50 μL of virus solution at a concentration of 1.2×10⁴ FFU/mL was added to each well; VGM-T alone was also added to cell control wells. The plates were incubated at 37° C. for 2 h, and then 100 μL of overlay was added to each well. The overlay contained equal volumes of 1.2% Avicel RC/CL (FMC Health and Nutrition, Philadelphia, PA, USA) and 2× MEM supplemented with 1 μg/mL TPCK-treated trypsin, 0.1% BSA, and 1% penicillin-streptomycin. After 18-22 h of incubation at 37° C., the overlay was removed and the cells were washed twice using PBS. Thereafter, cells were fixed with ice-cold 4% formalin at 4° C. for 30 min, followed by washing once with PBS and permeabilizing with 0.5% Triton X-100 at RT for 20 min. Monolayers were washed three times with PBS containing 0.1% Tween 20 (wash buffer) and incubated with a mouse-anti-IAV nucleoprotein monoclonal antibody at 37° C. for 1 h. ((Y. Huang et al, 2021, Vaccines, 9(7):793). After washing three times with wash buffer, the cells were incubated with a secondary antibody, goat anti-mouse peroxidase-labeled IgG (SeraCare, Inc., Milford, MA, USA), for 1 h at RT. Cells were then washed three times with wash buffer, and TrueBlue substrate (SeraCare, Inc., Milford, MA, USA) containing 0.03% H₂O₂ was added and incubated for 10 to 15 min at RT. The reaction was terminated by washing plates with distilled water five times. Plates were air-dried and foci were counted using a CTL BioSpot Analyzer with ImmunoCapture 6.4.87 software (CTL, Cleveland, OH, USA). The FRA titer was presented as the reciprocal of the highest dilution of sera corresponding to 50% focus reduction compared to the virus control wells minus the cell control wells.

All data are presented as absolute mean values ±standard errors of the means (SEM). One-way ANOVA was used to analyze the statistical differences among groups using GraphPad Prism 9 software (GraphPad, San Diego, CA, USA). A “p” value less than 0.05 was defined as statistically significant (*, p<0.05; **, p<0.01; ***, p<0.001; ****, p<0.0001).

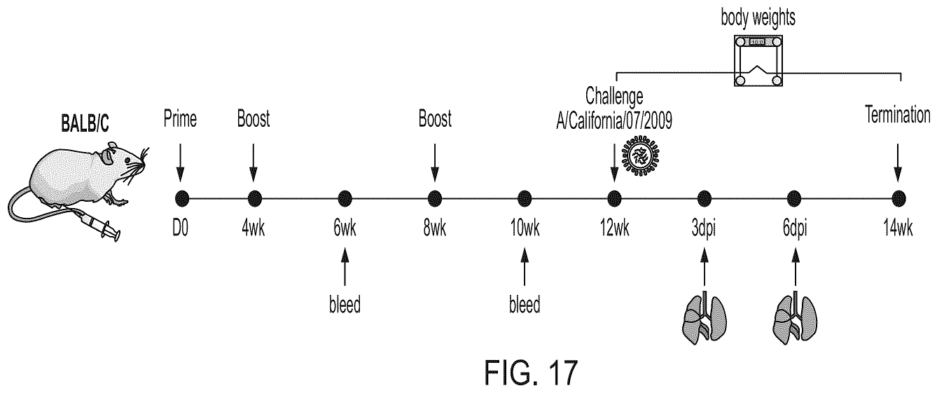

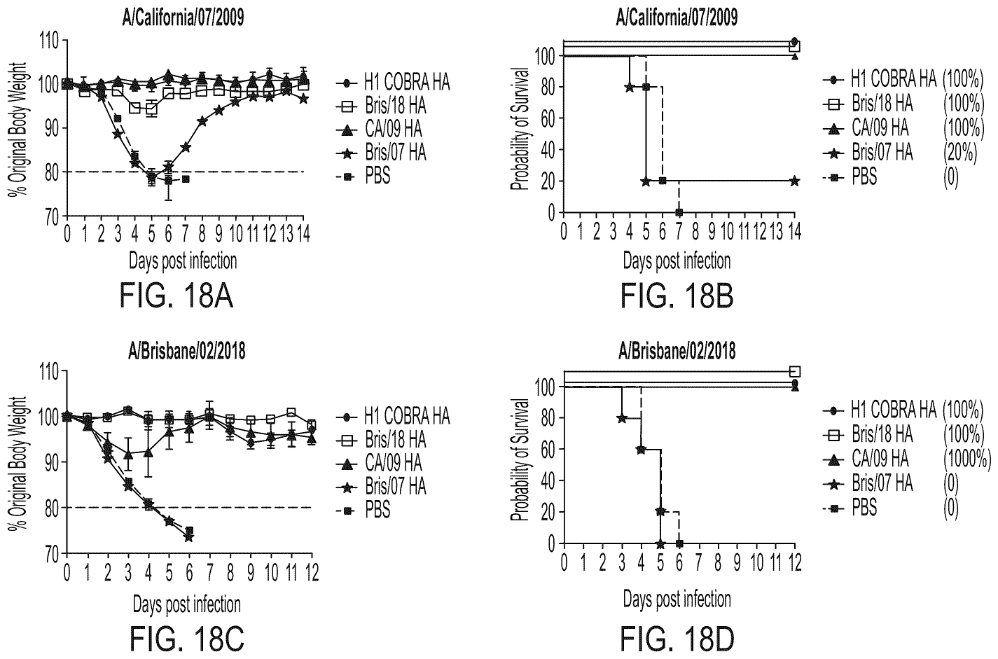

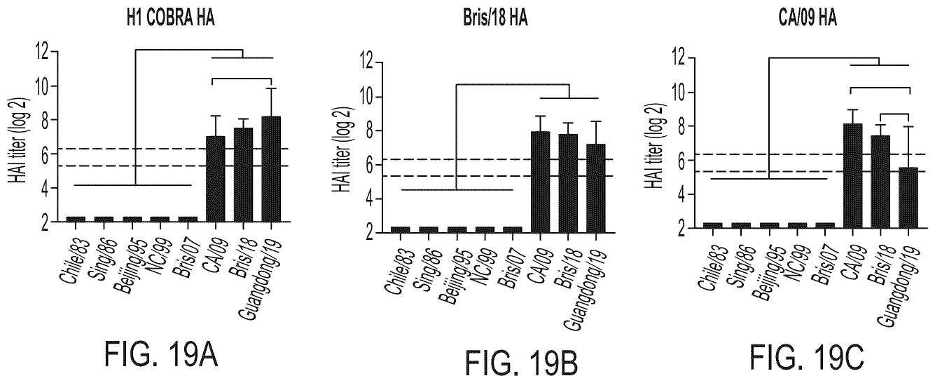

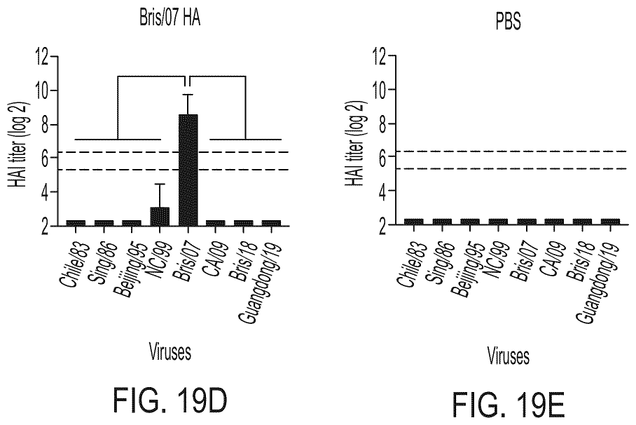

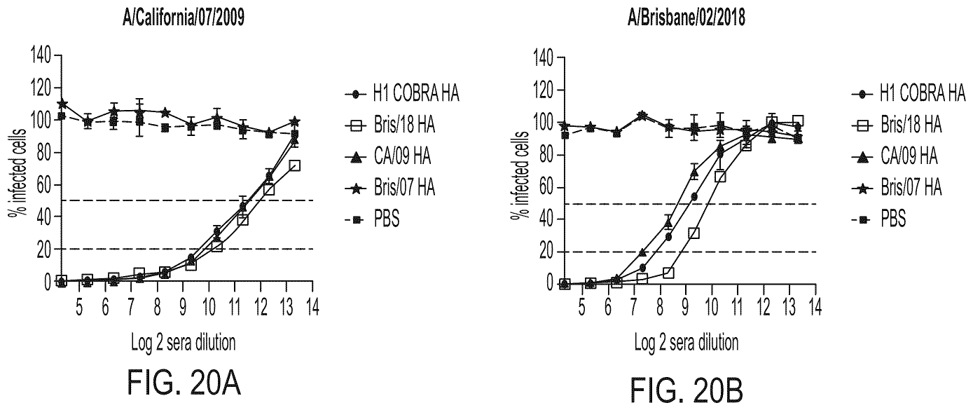

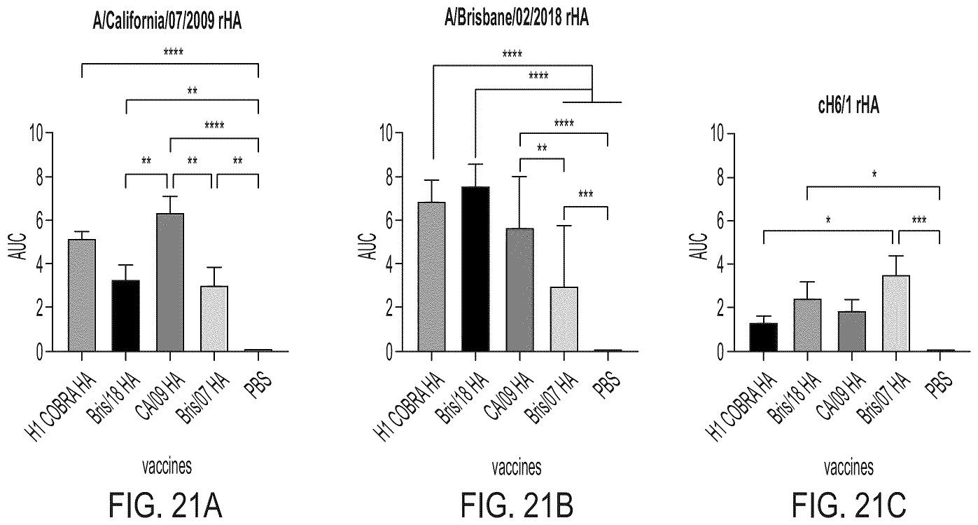

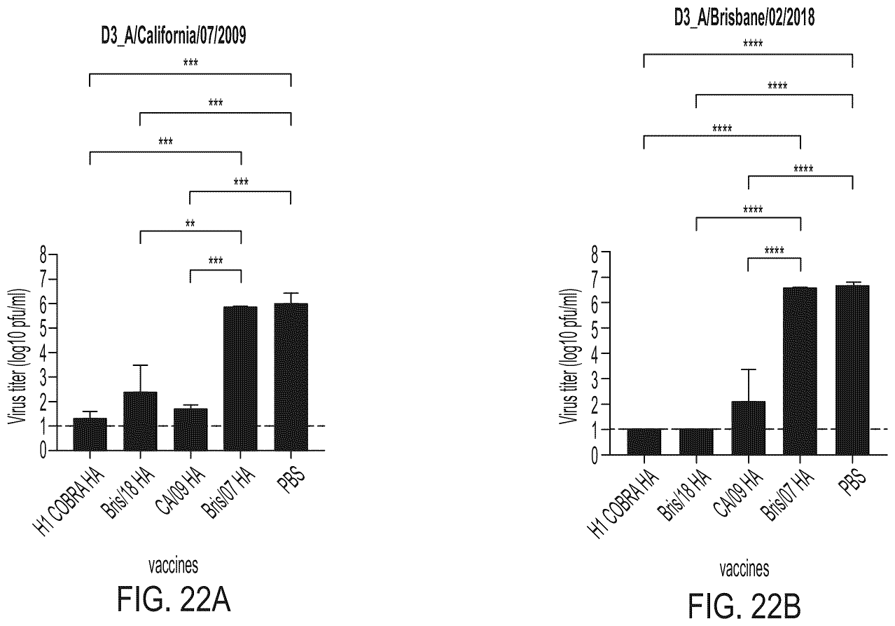

FIG. 17 depicts a timeline of the study described in Example 3. FIGS. 18A-18D present graphs demonstrating body weight and survival curves after influenza virus infection of the mice vaccinated with broadly-reactive, recombinant influenza HA immunogens according to the study described in Example 3 and in the timeline and protocol as illustrated in FIG. 17. The body weight and survival results provided in FIGS. 18A-18D showed that animals that had been vaccinated with broadly-reactive, recombinant influenza HA immunogens as described herein did not lose significant body weight and showed 100% survival following virus challenge. FIGS. 19A-19E show the serum HAI antibody titers in mice post vaccination against a panel of H1N1 viruses. Serum from animals that were vaccinated with VLPs encoding broadly-reactive, recombinant influenza H1N1 HA immunogen Y2 (e.g., SEQ ID NO: 15) or with H1N1 wildtype CA/09 or Bris/18 had high HAI titers against CA/09, Bris/18 and Guangdong/19 virus antigens post infection. FIGS. 20A and 20B show serum neutralizing antibody titers and neutralizing activity of the antibodies elicited in immunologically naïve Balb/c mouse sera post immunization/vaccination with a broadly reactive HA immunogenic polypeptide antigen as described herein (e.g., Y2 (SEQ ID NO: 15) or Y4 (SEQ ID NO: 17), (“H1 COBRA HA”), or with wild-type Bris/07, CA/09, or Bris/18 VLP immunogens/vaccines. Mice that were vaccinated with PBS and wild-type Bris/07 VLP immunogens did not have detectable neutralizing antibody titers against either the CA/09 virus (FIG. 20A) or Bris/18 virus (FIG. 20B). Mice immunized/vaccinated with the H1 COBRA HA VLP immunogens had high neutralizing antibody titers against both CA/09 and Bris/18 viruses. Antisera from the COBRA HA VLP vaccinated mice had a log 2 titer of 11.32 (50% inhibition) against CA/09 virus and a log 2 of 9.32 (50% inhibition) against the Bris/18 virus. FIGS. 21A-21C show total IgG antibody responses in mice that had been immunized/vaccinated with a broadly-reactive, recombinant influenza HA VLP immunogens (H1 Cobra HA, e.g., Y2 or Y4 rHA VLP immunogens), wild-type influenza rHA VLP immunogens, or PBS formulated with ADDAVAX™ adjuvant. IgG antibody titers were determined against (FIG. 21A): A/California/07/2009 rHA protein, (FIG. 21B): A/Brisbane/02/2018 rHA protein immunogen, or (FIG. 21C): cH6/1 HA protein immunogen: chimeric rHA with globular head from A/California/07/2009 HA and stalk form subtype H6 influenza virus rHA protein immunogen. Mice vaccinated with different VLPs had significantly higher IgG antibody titers against CA/09 HA than control mice that had received PBS. (FIG. 21A). Antisera from CA/09-rHA vaccinated mice had the highest total IgG titers against the homologously-matched CA/09 HA protein, while antisera from mice vaccinated with the H1 Cobra HA protein immunogens (e.g., Y2 or Y4 immunogens) had statistically similar IgG antibody titers compared to those in the CA/09 vaccinated mice. (FIG. 21A). Bris/18 rHA-vaccinated mice had lower IgG antibody titer compared to mice vaccinated with CA/09 rHA immunogen and mice vaccinated with the H1 Cobra HA protein immunogen (FIG. 21A), suggesting that the H1 Cobra HA protein antigens on the VLPs efficiently elicited IgG titers similar to those generated by the homologously matched CA/09 HA antigen. Antisera from mice vaccinated with the H1 Cobra HA protein immunogen VLPs (Y2 or Y4) had statistically similar total IgG titers against Bris/18 rHA compared with that in Bris/18- and in CA/09-vaccinated mice (FIG. 21B). FIGS. 22A and 22B show viral titers in the lung tissues of BALB/c and DBA/2J mice three days after infection with H1N1 A/California/07/2009 virus or A/Brisbane/02/2018 virus. BALB/c mice were intramuscularly vaccinated with a broadly reactive, HA immunogenic polypeptide vaccine (H1 Cobra HA VLP vaccine), e.g., Y2 or Y4 immunogens, or with wild-type HA VLP vaccines (e.g., Bris/18 HA, CA/09 HA, Bris/07 HA), (x-axis), and then challenged with H1N1 A/California/07/2009 virus (FIG. 22A) at week 12 post vaccination. Lung samples (n=3/group) were collected at day 3 post infection and lung viral titers were determined. Another set of DBA/2J mice that had been immunized/vaccinated with the same vaccines as noted above, delivered in a rHA format, were challenged with A/Brisbane/02/2018 virus at week 12 post vaccination (FIG. 22B). Lung samples (n=3) were harvested on day 3 post infection, and lung viral titers were determined (FIG. 22B). It was determined from this study that for the A/California/07/2009 (“CA/09”) virus challenge, the PBS-vaccinated mice had the highest lung viral titers on day 3 post-infection, and that mice vaccinated with Bris/07 HA VLPs had statistically similar lung viral titers compared to the PBS-vaccinated animals. The mice vaccinated with the broadly reactive, HA immunogenic polypeptide VLP vaccine (e.g., Y2) had the lowest lung viral titers, which were statistically similar to that of CA/09-vaccinated mice (FIG. 22A). For the A/Brisbane/02/2018 (“Bris/18”) virus challenge, the PBS-vaccinated mice had the highest lung viral titers on day 3 post-infection, and the mice vaccinated with the broadly reactive, HA immunogenic polypeptide VLP vaccine (e.g., Y2) or with Bris/18 had essentially no viral titer in their lungs, while mice vaccinated with CA/09 had significantly lower lung viral titer than that in PBS-vaccinated mice. Mice vaccinated with Bris/07 had viral titers that were similar to those in the PBS-vaccinated mice (FIG. 22B). These results demonstrated that animals immunized/vaccinated with the broadly reactive, HA H1N1 immunogenic polypeptide vaccines decrease viral loads in the lungs after challenge and infection with influenza viruses.

As described in the Examples supra, the non-naturally occurring, broadly reactive, H1 and H3 immunogenic polypeptides used as vaccines as described herein vaccine (e.g., Y2, J4, and NG2), were evaluated in mice that had been previously exposed to influenza A(H1N1) and A(H3N2) viruses, i.e., preimmune animals. While the impact of preimmunity on influenza vaccination has been understudied, such an evaluation is critical, because most humans are infected with influenza viruses before the age of five years. Therefore, the majority of the targeted vaccine population will already possess an immune history to one or both of these virus subtypes prior to vaccination. In the preimmune model, the J4 immunogen had improved HAL reactive antibody breadth compared to other vaccine candidates in the influenza-naive model. Preimmune mice vaccinated with the J4 immunogen also generated serum antibodies with HAI activity against Switz/13, Kan/17, Tx/17, and HK/19 virus types, which were not present in influenza-naive J4 HA vaccinated mice. A similar expansion of breadth occurred in the preimmune mice vaccinated with Sing/16, which generated serum antibodies with HAT activity against HK/19 in the preimmune model that were not present in the influenza-naive mice.

Without wishing to be bound by theory, seasonal influenza vaccination in the context of preimmunity is strongly biased by contributions from the immune memory compartment and primarily stimulates memory B cells leading to a transient surge in the number of antibody-secreting plasmablasts. Therefore, the observed expansion of reactive antibodies (antibody breadth) is likely due to memory B cell responses that recognize shared epitopes between the rHA vaccine antigens and the HA protein of 1H3N2 priming strain, as broad HA group-level immune memory arises when lymphocytes target conserved HA epitopes. Furthermore, preimmune imprinting induces immune memory to both conserved and variable sites on different influenza virus antigens. Thus, the shared HA epitopes between vaccine antigen and the priming strain may also be present on the H3N2 viruses in the historical HAI panel. Upon vaccine-induced immune recall, specific memory B cells are stimulated in preimmune animals to produce HAI reactive antibodies that are not elicited by the vaccine in the absence of influenza preimmunity.

### OTHER EMBODIMENTS

From the foregoing description, it will be apparent that variations and modifications may be made to various aspects and embodiments described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A non-naturally occurring and immunogenic influenza virus antigen or a polynucleotide encoding the antigen, the antigen comprising or consisting of an amino acid sequence that is at least 95% identical to an amino acid sequence of a hemagglutinin (HA) protein antigen of any one of SEQ ID NOS: 1-17, or an immunogenic portion thereof.

2. The influenza virus antigen of claim 1, wherein the influenza virus is an H1 or H3 influenza virus.

3. A virus-like particle (VLP) comprising the influenza virus antigen or a polynucleotide encoding the influenza virus antigen of claim 1.

4. A non-naturally occurring immunogen capable of generating an immune response against present and future influenza virus strains; wherein the immunogen comprises an amino acid sequence that is at least 95% identical to an amino acid sequence of a hemagglutinin (HA) antigen as set forth in SEQ ID NOS: 1-17.

5. The virus antigen of claim 1, which generates an immune response comprising the production of neutralizing antibodies.

6. An immunogenic composition or vaccine comprising the immunogen of claim 1 or a virus-like particle comprising the immunogen of claim 1.

7. A method of treating, protecting, and/or generating an immune response in a subject, the method comprising administering to the subject an effective amount of the virus antigen, VLP, or immunogen of claim 6.

8. The method of claim 7, wherein the subject is infected with influenza virus, or is at risk of or susceptible to infection by influenza virus.

9. The method of claim 7, wherein the subject generates an immune response comprising the production of neutralizing antibodies, a cellular immune response, and/or the production of T-lymphocytes.

10. The polynucleotide of claim 1, wherein the polynucleotide is RNA or DNA.

11. A pharmaceutical composition comprising a pharmaceutically acceptable carrier, diluent, or excipient and the polynucleotide of claim 1 or a virus like particle comprising the polynucleotide of claim 1.

12. A monovalent immunogen comprising a non-naturally occurring and immunogenic influenza virus antigen of claim 1.

13. The monovalent immunogen of claim 12, wherein the immunogenic influenza virus antigen comprises Y2 antigen comprising the sequence set forth in SEQ ID NO: 15; J1 antigen comprising the sequence set forth in SEQ ID NOS: 3, 7, or 9; J2 antigen comprising the sequence set forth in SEQ ID NO: 4; J3 antigen comprising the sequence set forth in SEQ ID NO: 5; J4 antigen comprising the sequence set forth in SEQ ID NOS: 6 or 8; NG1 antigen comprising the sequence set forth in SEQ ID NO: 11; NG2 antigen comprising the sequence set forth in SEQ ID NOS: 2 or 12; or NG3 antigen comprising the sequence set forth in SEQ ID NO: 13.

14. A multivalent immunogen comprising at least two of the non-naturally occurring and immunogenic influenza virus antigens of claim 1.

15. The multivalent immunogen of claim 14, wherein the immunogen is bivalent and comprises a combination of Y2 antigen comprising the sequence set forth in SEQ ID NO: 15 and J4 antigen comprising the sequence set forth in SEQ ID NOS: 6 or 8, or a combination of Y2 antigen comprising the sequence set forth in SEQ ID NO: 15 and NG2 antigen comprising the sequence set forth in SEQ ID NOS: 2 or 12.

16. The multivalent immunogen of claim 14, wherein the immunogen comprises eight of the non-naturally occurring and immunogenic influenza virus antigens.

17. A virus particle or virus-like particle (VLP) or pharmaceutical composition comprising one or more polynucleotides encoding the immunogenic influenza virus antigens of claim 14.

18. A method of generating an immune response and/or treating or protecting a subject from disease and/or the symptoms thereof, caused by influenza virus infection, the method comprising administering to the subject an effective amount of the pharmaceutically acceptable composition of claim 17.

19. The method of claim 18, wherein the influenza virus is an H1 and/or H3 influenza virus.

20. The method of claim 18, wherein the subject generates an immune response comprising the production of neutralizing antibodies, a cellular immune response, and/or the production of T-lymphocytes.
